(19) United States
(12) Patent Application Publication
(10) Pub. No.: US 2026/0109938 A1
(43) Pub. Date: Apr. 23, 2026

(54) GENETIC CODES

(71) Applicant: United Kingdom Research and Innovation, Swindon, Wiltshire (GB)

(72) Inventors: Jason CHIN, Swindon, Wiltshire (GB); Jerome ZURCHER, Swindon, Wiltshire (GB); Wesley ROBERTSON, Swindon, Wiltshire (GB)

(21) Appl. No.: 18/993,813

(22) PCT Filed: Jul. 19, 2023
(86) PCT No.: PCT/EP2023/070049

§ 371 Date: Jan. 13, 2025

(30) Foreign Application Priority Data
Jul. 19, 2022 (GB) 2210580.3
Oct. 18, 2022 (GB) 2217789.3

(51) Int. Cl. C12N 1/20; C12N 1/36; C12N 15/70; C12P 1/04; C12R 1/19

(52) U.S. Cl. CPC C12N 1/20; C12N 1/36; C12N 15/70; C12P 1/04; C12R 2001/19

(57) ABSTRACT

Provided are cells that are resistant to mobile genetic elements or horizontal gene transfer, and methods for obtaining said cells. Also provided are methods for preventing the horizontal transfer of genetic information between a mobile genetic element and a first cell, cells making use of new genetic codons schemes and related subject matter, kits comprising mutually orthogonal cells, and mobile genetic elements. Also provided are methods of altering the susceptibility of a gene to mutations that alter the encoded amino acid sequence, methods for evolving or improving a protein, and methods for rendering a target gene more resistant to mutation. Additionally provided are uses of the cells for making polymers and methods comprising using the cells for making polymers.

### CROSS-REFERENCE TO RELATED APPLICATIONS

This application is a national phase filing under 35 U.S.C. § 371 of International PCT Application No. PCT/EP2023/070049, filed Jul. 19, 2023, which claims the benefit of priority to United Kingdom Application No. 2210580.3 filed on Jul. 19, 2022, and United Kingdom Application No. 2217789.3 filed on Oct. 18, 2022, the content of each of which is hereby incorporated by reference in its entirety.

### INCORPORATION BY REFERENCE OF SEQUENCE LISTING

The instant application contains a Sequence Listing which has been submitted electronically in XML format and is hereby incorporated by reference in its entirety. Said XML copy, created on Dec. 5, 2025 is named 51689-019001_Sequence_Listing_12_5_25 and is 23,934,723 bytes in size.

### FIELD OF THE INVENTION

Provided herein are cells that are resistant to mobile genetic elements or horizontal gene transfer, and methods for obtaining said cells. Also provided are methods for preventing the horizontal transfer of genetic information between a mobile genetic element and a cell, cells making use of new genetic codons schemes and related subject matter, kits comprising mutually orthogonal cells, and mobile genetic elements. Also provided are methods of altering the susceptibility of a gene to mutations that alter the encoded amino acid sequence, methods for evolving or improving a protein, and methods for rendering a target gene more resistant to mutation. Additionally provided are uses of the cells for making polymers and methods comprising using the cells for making polymers.

### BACKGROUND OF THE INVENTION

The near-universal genetic code defines the correspondence between codons in genes and amino acids in proteins (1, 2). Because all forms of life use essentially the same genetic code, evolutionary innovation can be shared—via horizontal gene transfer (HGT)—between organisms (3, 4). The sharing of genetic information between organisms is a major driver of evolution in prokaryotes and some eukaryotes (5).

However, the near-universal genetic code is also a liability for organisms; mobile genetic elements (or selfish genetic elements)—including transposons, viruses and plasmids—exploit the universality of the code, and co-opt the host cell's machinery to read their genes and propagate themselves at the expense of host organisms. There is a clear tension between maintaining a common genetic code, to allow the acquisition of beneficial innovation through HGT, and excluding selfish genetic elements that exploit the common code for their own ends (3, 6).

Several deviations from the standard genetic code have been documented in mitochondria and chloroplasts, and the vast majority of characterized code reassignments involve stop codons (7-9). Known sense codon reassignments in the nuclear genome are rare. The ‘CTG yeast’ decode the CUG codon (which encodes leucine in the standard code) primarily as serine (97%, with the remaining 3% still assigned to leucine) (10). Viruses for the CTG yeasts are essentially unknown, suggesting that sense codon reassignment may protect against viruses (11). There are no experimentally validated examples of sense codon reassignment in bacteria, though recent work provides computational evidence for reassignment of arginine codons in bacilli (12).

Genome synthesis (13-15) and editing provides the opportunity to rewrite the genetic code of organisms (15-17). We synthesized a 4 Mb Escherichia coli genome in which we compressed the genetic code by removing all annotated occurrences of the TCG and TCA sense codons that encode serine, and the TAG stop codon; this created a new strain, Syn61 (15). We then further evolved the strain and deleted the genes for the tRNAs that decode TCG and TCA codons (serU, tRNA_{CGA}^{Ser} and serT, tRNA_{UGA}^{Ser}) and the gene for RF-1 (prfA) that terminates protein synthesis at the TAG stop codon. The resulting organism, Syn61Δ3, cannot read all the codons in the near universal genetic code and therefore cannot read horizontally transferred genes containing the codons deleted from its genome, as exemplified by resistance to a range of bacteriophage (18).

It has been widely hypothesized that refactoring the structure of the genetic code, through the reassignment of sense codons to distinct canonical amino acids, would create organisms with new properties, and could create a genetic firewall to limit the escape of genetic information from synthetic organisms to natural organisms (4, 6, 19, 20). However, these hypotheses remain untested.

### SUMMARY OF THE INVENTION

In the experiments disclosed herein, the genetic code of a synthetic E. coli strain is refactored to exhibit semantic- and functional orthogonality with respect to the universal genetic code, allowing for the creation of orthogonal horizontal gene transfer systems.

In an aspect, there is provided a cell that: comprises a genome wherein at least a first type of sense codon has been recoded such that a first endogenous tRNA is dispensable; does not express the first endogenous tRNA; expresses a first modified tRNA capable of decoding the first type of sense codon, wherein the first modified tRNA is charged with a first amino acid that is not a naturally cognate amino acid for the first type of sense codon; and comprises a gene required for viability, wherein the gene comprises at least one occurrence of the first type of sense codon and the cell is viable when the first type of sense codon in said gene is decoded as the first amino acid.

In another aspect, there is provided a cell that: comprises a genome wherein a first type of sense codon and a second type of sense codon have been recoded such that a first endogenous tRNA and a second endogenous tRNA are dispensable; does not express the first endogenous tRNA and the second endogenous tRNA; expresses a first anticodon-swapped tRNA derived from a naturally occurring first parent tRNA, wherein the first anticodon-swapped tRNA is charged with a first amino acid and the first parent tRNA is an isoacceptor for the first amino acid, and wherein the first amino acid is not a naturally cognate amino acid for the first type of sense codon; and expresses a second anticodon-swapped tRNA derived from a naturally occurring second parent tRNA, wherein the second anticodon-swapped tRNA is charged with a second amino acid and the second parent tRNA is an isoacceptor for the second amino acid, and wherein the second amino acid is not a naturally cognate amino acid for the second type of sense codon; wherein the first and/or second modified tRNA cannot decode any type of codon apart from the first type of sense codon and/or the second type of sense codon.

In another aspect, there is provided a cell that: comprises a genome wherein a first type of sense codon and a second type of sense codon have been recoded such that a first endogenous tRNA and a second endogenous tRNA are dispensable; does not express the first endogenous tRNA and the second endogenous tRNA; expresses a first modified tRNA capable of decoding the first type of sense codon, wherein the first modified tRNA is charged with a first amino acid that is not a naturally cognate amino acid for the first type of sense codon; and expresses a second modified tRNA capable of decoding the second type of sense codon, wherein the second modified tRNA is charged with a second amino acid that is not a naturally cognate amino acid for the second type of sense codon; wherein: i) the first amino acid is alanine and the second amino acid is alanine; ii) the first amino acid is alanine and the second amino acid is histidine; iii) the first amino acid is alanine and the second amino acid is leucine; iv) the first amino acid is alanine and the second amino acid is proline; v) the first amino acid is histidine and the second amino acid is alanine; vi) the first amino acid is histidine and the second amino acid is histidine; vii) the first amino acid is histidine and the second amino acid is leucine; viii) the first amino acid is histidine and the second amino acid is proline; ix) the first amino acid is leucine and the second amino acid is alanine; x) the first amino acid is leucine and the second amino acid is histidine; xi) the first amino acid is leucine and the second amino acid is proline; xii) the first amino acid is proline and the second amino acid is alanine; xiii) the first amino acid is proline and the second amino acid is histidine; xiv) the first amino acid is proline and the second amino acid is leucine; or xv) the first amino acid is proline and the second amino acid is proline.

In another aspect, there is provided a cell with increased resistance to horizontal gene transfer or mobile genetic elements, wherein the cell has been modified to reassign at least one type of sense codon to an amino acid not associated with the sense codon in the canonical genetic code, and the cell comprises a gene required for viability that is functional when decoded according to the reassigned genetic code and is not functional when decoded according to the canonical genetic code.

In another aspect, there is provided a method of increasing the resistance of a cell to mobile genetic elements or horizontal gene transfer, wherein the cell has been modified to reassign at least one type of sense codon to an amino acid not associated with the sense codon in the canonical genetic code, said method comprising: modifying a gene required for viability to include at least one occurrence of the reassigned sense codon, wherein the cell is viable if the reassigned sense codon in said gene is decoded as the reassigned amino acid, and the cell is not viable if the reassigned sense codon in said gene is decoded according to the canonical genetic code, or wherein the reassigned sense codon in said gene at least partially contributes to a loss of viability if decoded according to the canonical genetic code.

In another aspect, there is provided a kit comprising a first cell recoded according to a first orthogonal coding scheme and a second cell recoded according to a second orthogonal coding scheme, where the first and second coding schemes are mutually orthogonal.

In another aspect, there is provided a mobile genetic element recoded according to an orthogonal coding scheme.

In another aspect, there is provided a method of preventing the horizontal transfer of genetic information between a mobile genetic element and a first cell, the method comprising incubating the mobile genetic element and the first cell, wherein the mobile genetic element is a mobile genetic element as disclosed herein, and the first cell includes tRNAs that decode codons according to the canonical genetic code or according to a coding scheme that is orthogonal to that of the mobile genetic element.

In another aspect, there is provided a method of altering susceptibility of a gene to mutations that alter the encoded amino acid sequence, the method comprising: i) identifying a target gene; and ii) incubating a cell comprising the target gene, wherein the cell comprises a tRNA capable of decoding at least one sense codon to a reassigned amino acid.

In an additional aspect, there is provided use of a cell disclosed herein for the production of a polymer. In an embodiment, there is provided a method for making a polymer, the method comprising: culturing a cell disclosed herein, providing the cell with a nucleic acid sequence encoding the polymer, and obtaining the polymer.

### BRIEF DESCRIPTION OF THE DRAWINGS

FIG. 1. Compressed genetic codes are non-orthogonal. (A) The relationship between the TCG and TCA codons in genes, the decoders for these codons in cells with wild-type (WT) decoding and Syn61Δ3 decoding (Δ3), and the corresponding protein sequence synthesized. The anticodon of the tRNAs that read TCG or TCA codons is indicated (decoder). The amino acid (aa) used by the tRNA is indicated. Grey for a decoder indicates that the tRNA is loaded with serine. Grey for a codon indicates that the codon is within a non-codon compressed gene and its decoding as serine will make the correct protein sequence. Pink for a decoder/amino acid pair indicates that the tRNA is deleted. Pink for a codon indicates the codon in absent from the gene, as the gene has been designed with codon compression. (B) Functional assessment of wild type (SpecR WT) and codon compressed spectinomycin resistance (recSpecR (ΔTCG, TCA)) genes in cells that use the full complement of tRNAs to decode all the codons in the reading frame (Syn61 WT, left panel) and cells in which the tRNAs that decode TCG and TCA codons (Syn61Δ3, right panel) have been deleted. Cells were spotted on Agar plates in the presence or absence of spectinomycin and incubated overnight. Growth of cells in the presence of spectinomycin indicates that the indicated SpecR gene is functional in the indicated strain. (C, D) Predicted protein synthesis and horizontal gene transfer outcomes from mobile genetic elements and recipient cells with the indicated decoders, and codons in essential genes. (C) A mobile genetic element encoding its genes according to the canonical genetic code, where TCG and TCA encode serine, cannot be horizontally transferred to Syn61Δ3 cells that have no decoders for TCG and TCA codons. Translation will stall at TCG and TCA codons, and no full-length protein will be synthesized from the essential genes within the mobile genetic element that contain TCG and TCA codons. (D) A mobile genetic element encoding its genes according to the canonical genetic code that also carries a gene for a tRNA decoding TCG and TCA codons can be horizontally transferred to Syn61Δ3 cells. The tRNA encoded on the mobile genetic element can rescue decoding of TCG and TCA codons within essential genes in the mobile genetic element to make the correct protein. (E) Transfer of and tRNA encoding mobile genetic elements through conjugation; Colony count indicates successful transconjugants received from ˜10⁶ cells. A WT mobile genetic element (F WT) can be transferred into cells with a WT translational machinery (Syn61 WT) but not into cells that lack tRNAs decoding TCG and TCA (Syn61Δ3). A WT mobile genetic element that encodes for a tRNA decoding TCG and TCA codons as serine (F (WT+serT)) can be transferred to both Syn61 WT and Syn61Δ3.

FIG. 2. Sense codon reassignment generates new genetic codes. (A) Total synthesis of a codon compressed genome followed by tRNA and release factor deletion yielded Syn61Δ3. The discovery of tRNAs that direct the incorporation of distinct natural amino codes. (B) Isoacceptor tRNAs for the indicated amino acids with anticodons altered to the Watson-Crick complement of TCG or TCA codons were introduced into cells in the indicated pair-wise combinations. We read out the identity of the amino acid incorporated into each codon using GFP genes with TCG or TCA codons at position 3 and electrospray ionization mass spectrometry (ESIMS). When pairs of isoacceptors for distinct amino acids were used, each codon led to the specific incorporation of the amino acid attached to the Watson-Crick paired isoacceptor. The secondary peak measured in the proline incorporations results from incomplete methionine cleave at the N-terminus. A complete list of found and expected masses are provided in Data File S1. (C) Sixteen new genetic codes in which TCG and TCA codons are reassigned to Ala, His, Leu and Pro.

FIG. 3. Semantic orthogonality in genetic systems. (A) The relationship between the TCG and TCA codons in genes, the decoders for these codons in cells with wild-type (WT) decoding and decoding by tRNA^{Ala}_{CGA}, tRNA^{His}_{UGA} in Syn61Δ3, and the corresponding protein sequence synthesized. The anticodon of the tRNAs that read TCG or TCA codons is indicated (decoder). The amino acid (aa) used by the tRNA is indicated. Grey for a codon indicates that its decoding as serine will make the correct protein sequence. Yellow for a codon indicates that its decoding as alanine will make the correct protein sequence. Green for a codon indicates that its decoding as histidine will make the correct protein sequence. (B) Functional assessment of SpecR WT (which uses the natural genetic code) and O-SpecR (TCG-Ala, TCA-His) which is codons compressed according the Syn61 recoding scheme and has Ala codons replaced with TCG and His codons replaced with TCA. The genes are read in cells with a WT translational machinery (Syn61 WT), and cells where TCG is decoded as Ala and TCA is decoded as His (Syn61Δ3 (tRNA_{CGA}^{Ala}, tRNA_{UGA}^{His})). Cells were spotted on Agar plates in the presence or absence of spectinomycin and incubated overnight. Growth of cells in the presence of spectinomycin indicates that the indicated SpecR gene is functional in the indicated strain.

FIG. 4. Orthogonal and mutually orthogonal horizontal gene transfer systems. (A) Horizontal gene transfer between the two bacterial strains in prohibited (dashed grey arrows), while horizontal gene transfer among cells that share a common genetic code is possible (solid arrows). (B) Orthogonal horizontal transfer of mobile genetic elements. Colony count indicates the number of transconjugants received from ˜10⁶ donor cells bearing the indicated mobile genetic element. A WT mobile genetic element (F WT) was transferred into cells with WT translational machinery (Syn61 WT) but not into cells where TCG is reassigned to alanine and TCA is reassigned to histidine (Syn61Δ3 (tRNA_{CGA}^{Ala}, tRNA_{UGA}^{His})). An orthogonal mobile genetic element (OF1) was transferred into Syn61Δ3 (tRNA_{CGA}^{Ala}, tRNA_{UGA}^{His}) but not into Syn61 WT. (C) Mutually orthogonal horizontal genetic systems. Colony count indicates successful transconjugants received from ˜10⁶ donor cells bearing the indicated mobile genetic element. The orthogonal mobile genetic element (O-F2) was exclusively be transferred into cells where TCG is reassigned to histidine and TCA is reassigned to alanine (Syn61Δ3 (tRNA_{CGA}^{His}, tRNA_{UGA}^{Ala})); O-F2 was not transferred into Syn61Δ3 (tRNA_{CGA}^{Ala}, tRNA_{UGA}^{His}) or Syn61 WT. Neither F WT nor O-F1 can be transferred to (Syn61Δ3 (tRNA_{CGA}^{His}, tRNA_{IGA}^{Ala}). (D) extends the experiments illustrated in (C) and demonstrates similar specificities for O-F3 and O-F4.

FIG. 5. Orthogonal code-locking blocks invading codes. (A, B) Predicted protein synthesis and horizontal gene transfer outcomes from mobile genetic elements and recipient cells with the indicated decoders, and codons in essential genes. (A) Transfer of a WT mobile genetic element that encodes for a tRNA decoding TCG and TCA codons as Ser into a cell where TCG is reassigned to Ala and TCA is reassigned to His. Essential genes in the WT mobile genetic element, containing TCG and TCA codons, will be mis-synthesized, with each TCG and TCA codon in the gene being stochastically decoded as Ser or His/Ala. This is predicted to attenuate horizontal gene transfer. (B) Transfer of a WT mobile genetic element that encodes for a tRNA decoding TCG and TCA codons as serine into a cell where TCG is reassigned to Ala and TCA is reassigned to His. Essential genes in the WT mobile genetic element, containing TCG and TCA codons, will be mis-synthesized, with each TCG and TCA codon in the gene being stochastically decoded as Ser or His/Ala. In addition, essential genes in the host cell—in which TCG is used to encode Ala and TCA is used to encode His—will be mis-synthesized. This is predicted to ablate horizontal gene transfer. (C) Horizontal gene transfer is ablated in cells that use orthogonal genetic codes in essential genes of mobile genetic elements and the recipient cell. F (WT+serT) was used as the mobile genetic element in all experiments. Colony count indicates successful transconjugants received from ˜10⁶ cells. Recipient cells and spectinomycin resistance gene variant (SpecR gene) in the recipient cell is indicated. Correctly reading the indicated SpecR gene, in the recipient cell, is made essential by addition of spectinomycin. (D) T4-like phage encoding a seryl-tRNA_{UGA} infect Syn61Δ3, but not cells bearing orthogonal genetic codes. Plaque count indicates the number of successfully replicating phage obtained from infection with 1.1×10¹⁰ PFU/mL (phage 12) and 7.5×10⁹ PFU/mL (phage 6). Cells contain cognate spectinomycin resistance genes, as in C; all experiments were performed in the presence of spectinomycin.

FIG. 6 (FIG. S1). Compressed Genetic Codes are non-orthogonal. Functional assessment of wild type (HygR WT) and codon compressed hygromycin resistance (recHygR (ΔTCG, TCA)) genes in cells that use the full complement of tRNAs to decode all the codons in the reading frame (Syn61 WT, left panel) and cells in which the genes for the tRNAs that decode TCG and TCA codons (Syn61Δ3, right panel) have been deleted. Cells were spotted on agar plates in the presence or absence of hygromycin and incubated overnight. Growth of cells in the presence of hygromycin indicates that the given HygR gene is functional in the given strain.

FIG. 7 (FIG. S2). tRNA import breaks genetic isolation. A WT mobile genetic element (F WT) encoding a chloramphenicol resistance gene and the lux operon, encoding the enzymes for the synthesis of luciferin, was conjugated from Donor cells (Syn61 WT) into Recipient cells (Syn61Δ2: this is a strain derived from Syn61Δ3 where prfA has been reintroduced via lambda red recombination), which contains a pSC101-Hyg plasmid encoding hygromycin resistance, and the pKW20 plasmid (35). After conjugation cells were selected on hygromycin and chloramphenicol containing agar plates so only cells that contain the pSC101 vector and F WT can survive. (A) Chemiluminescent image of selection plate from conjugation assay. The three colonies that survive on selection plates after conjugation luminesce as a result of luciferin production, indicating that they have received F WT. (B) Genotyping of two colonies picked from the selection plate (the third colony did not grow in liquid media). Controls included the Syn61 WT (containing genomically encoded prfA, serT and serU, these cells do not contain the pSC101-Hyg plasmid) and Syn61Δ3 (in which prfA, serT and serU have been deleted from the genome, these cells contain the pSC101-Hyg plasmid). Genotyping for the pSC101-Hyg plasmid confirmed that the clones were recipient cells. Genotyping the prfA locus revealed that both clones that survive selection contained the prfA gene at the endogenous genomic locus. Genotyping the serT locus reveals that both clones carried serT at the endogenous genomic locus. Genotyping the serU locus demonstrated that clones did not carry serU at the endogenous genomic locus. (C) Next generation sequencing (NGS) sequence alignments against reference genomes for the recipient and donor strain. We observed that at the serT locus the sequence of the clones matched the donor reference (no gaps or insertions are observed when aligned against the Syn61 WT sequence; insertions were observed when aligned against Syn61Δ3 sequence). At the serU locus however, the sequence of the clones matched the Syn61Δ3 sequence (insertion observed when aligned against Syn61 WT sequence. No gaps or insertions observed when aligned against the Syn61Δ3 sequence). Vertical coloured lines indicate a mismatch between the sequencing read and the reference. Grey shows paired end reads, where pairs are correctly oriented and positioned with respect to each other. Reads displayed in green/red indicate anomalous paired end reads, where pairs are misoriented or mispositioned with respect to each other. (D) Sequencing allowed us to define the origin of the genomic sequence for each clone; Syn61Δ3 (and therefore Syn61Δ2) contains mutations that result from its evolution from Syn61 WT, these mutations act as watermarks that allowed us to map whether DNA sequences are derived from the donor (Syn61 WT) or recipient (Syn61Δ2) genomes. We find that most of the genome is that of the recipient. However, over a stretch of ˜400 kb multiple segments of Donor DNA were integrated into the recipient genome. The pattern of genomic DNA integration was unique for each sequenced clone, but both clones contained the serT locus.

FIG. 8 (FIG. S3). Isoacceptor tRNAs with altered anticodons are active and specific. (A) Production of sfGFP-His6 from sfGFP3TCG or sfGFP3TCA gene in Syn61Δ3 cells harbouring the indicated isoacceptor tRNA chimera with a CGA or UGA anticodon. serT and serU decode both TCG and TCA codons, while the chimeric isoacceptors show specificity for codons with Watson-Crick complementarity at all three bases of the codon-anticodon interaction. The level of GFP produced by all chimeric tRNAs on their Watson Crick complement codon, for all but proM with a UGA anticodon, is at least comparable to the GFP produced from a WT GFP gene without TCG or TCA codon; this suggests that the chimeric isoacceptor tRNAs are efficient and specific. The protein yield for WT sfGFP is 17 mg/L. sfGFP-3-TCG/TCA expression is comparable to WT sfGFP for cognate decoders. (B) Chimeric isoacceptors retain specificity for aminoacylation by the amino acid specified by the parent isoacceptor. The identity of the amino acids incorporated in response to TCG or TCA at position 3 of sfGFP, in cells containing the chimeric tRNAs with an anticodon that was the Watson-Crick complement of the codon at position 3 of sfGFP, was confirmed by electrospray ionization mass spectrometry (ESI-MS). Only masses corresponding to the correct amino acid were detected. The secondary peak measured in the proline incorporations results from incomplete methionine cleave at the N-terminus. Expected and actual masses for expression of sfGFP3TCG; Expected mass sfGFP-3-Ser: 27755.13 Da, actual mass: 27756.00 Da. Expected mass sfGFP-3-Ala: 27739.13 Da, actual mass: 27740.60 Da. Expected mass sfGFP-3-His: 27805.19 Da, actual mass: 27806.20 Da. Expected mass sfGFP-3-Leu: 27781.21 Da, actual mass: 27782.00 Da. Expected mass sfGFP-3-Pro: 27765.17 Da, actual mass: 27765.40 Da. Expected and actual massed for expression of sfGFP3TCA; Expected mass sfGFP-3-Ser: 27755.13 Da, actual mass: 27756.00 Da. Expected mass sfGFP-3-Ala: 27739.13 Da, actual mass: 27741.20 Da. Expected mass sfGFP-3-His: 27805.19 Da, actual mass: 27805.60 Da. Expected mass sfGFP-3-Leu: 27781.21 Da, actual mass: 27782.00 Da. Expected mass sfGFP-3-Pro: 27765.17 Da, actual mass: 27765.20 Da.

FIG. 9 (FIG. S4). Semantic orthogonality in genetic systems. (A) Functional assessment of (SpecR WT), written in the canonical genetic code, and codon reassigned spectinomycin resistance (O-SpecR (His-TCG, Ala-TCA), O-SpecR (Ala-TCG, Leu-TCA), O-SpecR (Leu-TCG, Leu-TCA), O-SpecR (Pro-TCG, Leu-TCA), O-SpecR (Ala-TCG, Ala-TCA), O-SpecR (Ala-TCG, Pro-TCA)) genes in cells with a canonical decoding (Syn61 WT), and cells where TCG and TCA codons have been reassigned according to the appropriate code (Syn61Δ3 (tRNACGAHis, tRNAUGAAla), Syn61Δ3 (tRNACGAAla, tRNAUGALeu), Syn61Δ3 (tRNACGALeu, tRNAUGALeu), Syn61Δ3 (tRNACGAPro, tRNAUGALeu), Syn61Δ3 (tRNACGAAla, tRNAUGAAla), Syn61Δ3 (tRNACGAAla, tRNAUGAPro)). Cells were spotted on Agar plates in the presence or absence of spectinomycin and incubated overnight. Growth of cells in the presence of spectinomycin indicates that the indicated SpecR gene is functional in the indicated strain. (B) Functional assessment of HygR WT (which uses the natural genetic code) and O-HygR (TCG-Ala, TCA-His) which is codons compressed according the Syn61 recoding scheme and has Ala codons replaced with TCG and His codons replaced with TCA. The genes are read in cells with a WT translational machinery (Syn61 WT), and cells where TCG is decoded as Ala and TCA is decoded as His (Syn61Δ3 (tRNAcGAAla, tRNAuGAHis))

FIG. 10 (FIG. S5). The mutational landscape of WT and codon compressed genetic codes. Depiction of mutational landscapes for various amino acids in WT and codon compressed genetic codes. Amino acid for which mutational landscape is displayed are highlighted (serine in grey, alanine in yellow, histidine in green, leucine in orange, and proline in blue). Amino acids that are only one point mutation removed from the highlighted amino acid are connected via a line and marked.

FIG. 11 (FIG. S6). Refactored genetic codes alter the mutational landscape. Depiction of mutational landscapes for the refactored genetic codes created in this work. TCG reassignment is displayed on the left. TCA reassignment is displayed on top. For each code serine and the reassigned amino acids are highlighted. Amino acids that are only one point mutation removed from highlighted amino acids are connected via a line and marked.

FIG. 12 Raw spectra. (A) Complied mass spectra (pre-deconvolution) for sfGFP-3-TCG measurements (as displayed in FIG. 2B) are shown. The intensity at each mass is displayed as total ion count. (B) Complied mass spectra (pre-deconvolution) for sfGFP-3-TCA measurements (as displayed in FIG. 2B) are shown. The intensity at each mass is displayed as total ion count.

FIG. 13 The fidelity of TCG TCA decoding by isoacceptor tRNAs at position 11 of ubiquitin. Isoacceptor tRNAs for the indicated amino acids with anticodons altered to the Watson-Crick complement of TCG or TCA codons were introduced into Syn61Δ3 in the indicated pair-wise combinations. We read out the identity of the amino acid incorporated into each codon using ubiquitin genes with either TCG or TCA codons at position 11 and electrospray ionization mass spectrometry (ESI-MS). When pairs of isoacceptors for distinct amino acids were used, each codon led to the specific incorporation of the amino acid attached to the Watson-Crick paired isoacceptor. We calculated the minimum specificities for decoding TCG or TCA codons in the presence of both tRNA_{CGA}^{XXX} and tRNA_{UGA}^{YYY}, where XXX and YYY are distinct amino acids (Methods). We observed apparent incorporation of Ala at TCA in cells that contain tRNA_{CGA}^{Ala} and tRNA_{UGA}^{Leu}; we estimate ≥78.2% Leu incorporation at this codon; this may result in part from misacylation of tRNA_{UGA}^{Leu} and/or from competitive decoding of the TCA codon by tRNA_{CGA}^{Ala}. For all other spectra the specificity of decoding a codon with the correct vs incorrect anticodon ranged from ≥96% to ≥99.8%.

FIG. 14 Sense codon reassignment does not yield detectable off-target incorporation at TCT codons (A) Isoacceptor tRNAs for the indicated amino acids, with anticodons altered to the Watson-Crick complement of TCG or TCA codons, were introduced into cells in pair-wise combinations, as indicated. We read out the identity of the amino acid incorporated into a TCT codon at position 3 of GFP using electrospray ionization mass spectrometry (ESI-MS). Detected masses in the presence of all isoacceptors pairs correspond to the incorporation of serine at TCT codons. Expected mass for serine 27755.13 Da; alanine: 27739.13 Da; for histidine: 27805.19 Da; for leucine 27781.21 Da; for proline: 27765.17 Da. All measured masses (27754.5±0.5 Da) correspond to incorporation of serine at TCT. The limit of fidelity measurements (Methods) for these spectra range from 97.4% to 99.6%, and we do not observe peaks for incorporating amino acids other than serine. (B) Complied mass spectra (pre-deconvolution) for sfGFP-3-TCT measurements (as displayed in A) are shown. The intensity at each mass is displayed as total ion count.

FIG. 15 Sense codon reassignment does not yield detectable off-target incorporation at TCC codons (A) Isoacceptor tRNAs for the indicated amino acids, with anticodons altered to the Watson-Crick complement of TCG or TCA codons, were introduced into cells in pair-wise combinations, as indicated. We read out the identity of the amino acid incorporated into a TCC codon at position 3 of GFP using electrospray ionization mass spectrometry (ESI-MS). Detected masses in the presence of all isoacceptors pairs correspond to the incorporation of serine at TCC codons. Expected mass for serine 27755.13 Da; alanine: 27739.13 Da; for histidine: 27805.19 Da; for leucine 27781.21 Da; for proline: 27765.17 Da. All measured massed (27754.5±0.5 Da) correspond to incorporation of serine at TCC. The limit of fidelity measurements (Methods) for these spectra range from 98.0% to 99.5%, and we do not observe peaks for incorporating amino acids other than serine. (B) Complied mass spectra (pre-deconvolution) for sfGFP-3-TCC measurements (as displayed in A) are shown. The intensity at each mass is displayed as total ion count.

FIG. 16 Raw spectra Ubiquitin (A) Complied mass spectra (pre-deconvolution) for Ub-11-TCG measurements (as displayed in FIG. 13) are shown. The intensity at each mass is displayed as total ion count. (B) Complied mass spectra (pre-deconvolution) for Ub-11-TCA measurements (as displayed in FIG. 13) are shown. The intensity at each mass is displayed as total ion count.

FIG. 17 MS-MS of amino acids incorporated at position 11 of ubiquitin (A) Tandem mass spectrometry spectra of peptides covering position 11 of the ubiquitin protein, following digestion of ubiquitin protein expressed from Ub-11-TCG gene in the presence of tRNA_{UGA}^{Ser}. y-ions are labelled in red; b-ions in blue. The peptide sequence is displayed at the bottom of each spectrum. Serine (marked with a green asterisk) at position 5 of the peptide confirms correct decoding of the TCG codon in Ub-11-TCG by tRNA_{UGA}^{Ser}. (B) Tandem mass spectrometry spectra of peptides covering position 11 of the ubiquitin protein, following digestion of ubiquitin protein expressed from Ub-11-TCA gene in the presence of tRNA_{UGA}^{Ser}. y-ions are labelled in red; b-ions in blue. The peptide sequence is displayed at the bottom of each spectrum. Serine (marked with a green asterisk) at position 5 of the peptide confirms correct decoding of the TCA codon in Ub-11-TCA by tRNA_{UGA}^{Ser}.

FIG. 18 MS-MS of amino acids incorporated at position 11 of ubiquitin (A) Tandem mass spectrometry spectra of peptides covering position 11 of the ubiquitin protein, following digestion of ubiquitin protein expressed from Ub-11-TCG gene in the presence of tRNA_{CGA}^{Ala}. y-ions are labelled in red; b-ions in blue. The peptide sequence is displayed at the bottom of each spectrum. Alanine (marked with a green asterisk) at position 5 of the peptide confirms correct decoding of the TCG codon in Ub-11-TCG by tRNA_{CGA}^{Ala}. (B) Tandem mass spectrometry spectra of peptides covering position 11 of the ubiquitin protein, following digestion of ubiquitin protein expressed from Ub-11-TCA gene in the presence of tRNA_{UGA}^{Ala}. y-ions are labelled in red; b-ions in blue. The peptide sequence is displayed at the bottom of each spectrum. Alanine (marked with a green asterisk) at position 5 of the peptide confirms correct decoding of the TCA codon in Ub-11-TCA by tRNA_{UGA}^{Ala}.

FIG. 19 MS-MS of amino acids incorporated at position 11 of ubiquitin (A) Tandem mass spectrometry spectra of peptides covering position 11 of the ubiquitin protein, following digestion of ubiquitin protein expressed from Ub-11-TCG gene in the presence of tRNA_{CGA}^{His}. y-ions are labelled in red; b-ions in blue. The peptide sequence is displayed at the bottom of each spectrum. Histidine (marked with a green asterisk) at position 5 of the peptide confirms correct decoding of the TCG codon in Ub-11-TCG by tRNA_{CGA}^{His}. (B) Tandem mass spectrometry spectra of peptides covering position 11 of the ubiquitin protein, following digestion of ubiquitin protein expressed from Ub-11-TCA gene in the presence of tRNA_{UGA}^{His}. y-ions are labelled in red; b-ions in blue. The peptide sequence is displayed at the bottom of each spectrum. Histidine (marked with a green asterisk) at position 5 of the peptide confirms correct decoding of the TCA codon in Ub-11-TCA by tRNA_{UGA}^{His}.

FIG. 20 MS-MS of amino acids incorporated at position 11 of ubiquitin (A) Tandem mass spectrometry spectra of peptides covering position 11 of the ubiquitin protein, following digestion of ubiquitin protein expressed from Ub-11-TCG gene in the presence of tRNA_{CGA}^{Leu}. y-ions are labelled in red; b-ions in blue. The peptide sequence is displayed at the bottom of each spectrum. Leucine (marked with a green asterisk) at position 5 of the peptide confirms correct decoding of the TCG codon in Ub-11-TCG by tRNA_{CGA}^{Leu}. (B) Tandem mass spectrometry spectra of peptides covering position 11 of the ubiquitin protein, following digestion of ubiquitin protein expressed from Ub-11-TCA gene in the presence of tRNA_{UGA}^{Leu}. y-ions are labelled in red; b-ions in blue. The peptide sequence is displayed at the bottom of each spectrum. Leucine (marked with a green asterisk) at position 5 of the peptide confirms correct decoding of the TCA codon in Ub-11-TCA by tRNA_{UGALe}u.

FIG. 21. MS-MS of amino acids incorporated at position 11 of ubiquitin (A) Tandem mass spectrometry spectra of peptides covering position 11 of the ubiquitin protein, following digestion of ubiquitin protein expressed from Ub-11-TCG gene in the presence of tRNA_{CGA}^{Pro}. y-ions are labelled in red; b-ions in blue. The peptide sequence is displayed at the bottom of each spectrum. Proline (marked with a green asterisk) at position 5 of the peptide confirms correct decoding of the TCG codon in Ub-11-TCG by tRNA_{CGA}^{Pro}. (B) Tandem mass spectrometry spectra of peptides covering position 11 of the ubiquitin protein, following digestion of ubiquitin protein expressed from Ub-11-TCA gene in the presence of tRNA_{UGA}^{Pro}. y-ions are labelled in red; b-ions in blue. The peptide sequence is displayed at the bottom of each spectrum. Proline (marked with a green asterisk) at position 5 of the peptide confirms correct decoding of the TCA codon in Ub-11-TCA by tRNA_{UGA}^{Pro}.

FIG. 22 Screen of anticodon modified tRNAs (A) We expressed and purified ubiquitin-His6 with a TCG codon at position 11 (Ub-11-TCG-His6) in the presence of the indicated tRNAs with a CGA anticodon; expressions were performed in Syn61Δ3 and purified by Ni-NTA chromatography. To evaluate the amino acid specificity of the anticodon modified tRNAs we performed intact protein mass spectrometry. Expected and measured masses are indicated for each tRNA. The expected masses are for the amino acid of the parent isoacceptor, where the mass differs from this the mass for the closest canonical amino acid incorporation is also shown as an additional expected mass. The following tRNAs incorporated an amino acid distinct from that expected for the parent isoacceptor (or gave results that could not be unambiguously assigned to the amino acid of the parent isoacceptor) and were not considered further: AsnT, CysT, GlnV, LysQ, MetV, MetY, PheU, ValV, ValW. The following tRNAs incorporated the amino acid of the parent isoacceptor: ArgU, ArgX, ArgQ, ArgW, GltU, GlyU, HisR, ProK, ProL, ProM, ThrT, TrpT, TyrV. (B) For a subset of tRNAs that incorporated the amino acid of the parent isoacceptor, we expressed Ub-11-TCG-His6 in the presence of the indicated tRNAs with a CGA anticodon; expressions were performed in Syn61Δ3 and the lysates probed with anti His6 following SDS-PAGE (we replaced ThrT with ThrU). From these experiments we chose ProM and HisR as good candidates for further characterisation. In control experiments without tRNA expression we did not detect ubiquitin from Ub-11-TCG. SerU is a natural TCG decoder and is a positive control.

FIG. 23. Doubling times Doubling times for Syn61Δ3 in the presence of different pair-wise combinations of anticodon modified tRNAs were measured in 2×YT. The strains were no tRNA (−) or serT (tRNA_{UGA}^{Ser}) were present serve as controls. Most anticodon modified tRNAs lead to no or modest changes in doubling time of Syn61Δ3. To facilitate parallel measurements of doubling times were performed in a 96-well format in 200 uL volumes (Methods). For comparison, Syn61Δ3 (no tRNA (−) control) has a doubling time of 49.77±0.8 min, when grown in a shake flask

FIG. 24 Mutually orthogonal horizontal gene transfer systems Colony count indicates successful transconjugants received from ˜10⁶ donor cells bearing the indicated mobile genetic element. Mobile genetic elements—(F (WT (TCG-Ser, TCA-Ser)), O-F1 (TCG-Ala, TCA-His), O-F2 (TCG-His, TCA-Ala), O-F3 (TCG-Ala, TCA-Ala), O-F4 (TCG-Ala, TCA-Pro))—were exclusively transferred into cells with the cognate reassignment of TCG and TCA codons (Syn61 WT (tRNA_{CGA}^{Ser}, tRNA_{UGA}^{Ser}), Syn61Δ3 (tRNA_{CGA}^{Ala}, tRNA_{UGA}^{His}), Syn61Δ3 (tRNA_{CGA}^{His}, tRNA_{IGA}^{Ala}), Syn61Δ3 (tRNA_{CGA}^{Ala}, tRNA_{UGA}^{Ala}), Syn61Δ3 (tRNA_{CGA}^{Ala}, tRNA_{UGA}^{Pro})). No transconjugation was observed for non-cognate code/decoder systems.

FIG. 25 Orthogonal code-locking blocks invading codes Horizontal gene transfer of a WT mobile genetic element (F (WT+serT)) is ablated in cells that use a refactored genetic code in essential genes of mobile genetic elements and the recipient cell. Colony count indicates successful transconjugants received from ˜10⁶ cells. Recipient cells and hygromycin resistance gene variant (HygR gene) in the recipient cells indicated. Correctly reading the indicated HygR gene, in the recipient cell, is made essential by addition of hygromycin.

FIG. 26 Orthogonal code-locking blocks invading codes Horizontal gene transfer of a WT mobile genetic element (F (WT+serT)) is ablated in cells that use a refactored genetic code in essential genes of mobile genetic elements and the recipient cell. Colony count indicates successful transconjugants received from ˜10⁶ cells. Recipient cells and spectinomycin resistance gene variant (SpecR gene) in the recipient cells indicated. Correctly reading the indicated SpecR gene, in the recipient cell, is made essential by addition of spectinomycin.

FIG. 27. Whole genome sequencing of purified phage reveals a seryl-tRNA gene (A) We assessed thirty one phage enrichments from environmental samples for their ability to form plaques on a code compressed strain (Syn61Δ3) and a strain with a refactored and locked genetic code (Syn61Δ3 (tRNA_{CGA}^{Ala}, tRNA_{UGA}^{His})O-SpecR (Ala-TCG, His-TCA)) in the presence of spectinomycin. Thirteen of the environmental samples formed plaques on Syn61Δ3, while none of the samples formed plaques on the strain with a refactored genetic code (Methods). Two individual phage (named 6 and 12) were serially purified from single plaques in Syn61WT (as described in the Methods). High coverage NGS enabled de novo genome assembly for these phage. Their genome sizes were 164,924 and 167,593 respectively. Both phage belong to the Tequatrovirus genus (T4-like phage), showing >97% identity with Citrobacter phage ZZ23 (NC_054901) and Escherichia phage U115 (MZ753803) respectively. Both genomes contained a seryl-tRNA gene, encoding ΦtRNA_{UGA}^{Ser}. (B) The predicted secondary structure of the phage encoded seryl-tRNA (ΦtRNA_{UGA}^{Ser}) and serT (endogenous E. coli tRNA_{UGA}^{Ser} gene). All E. coli seryl-aaRS identity elements are present in ΦRNA_{UGA}^{Ser} (bases shown in red), consistent with the viral tRNA being a substrate for E. coli seryl-tRNA synthetase. The sequences shown in the figure are SEQ ID NO: 69 (endogenous E. coli tRNA_{UGA}^{Ser} gene) and SEQ ID NO: 70 (ΦtRNA_{UGA}^{Ser}). (C) Electron-microscopy images of isolated phages 6 and 12. Both phages showed a characteristic Myoviridae morphology, consistent with the phage genome sequences.

FIG. 28 Providing tRNA_{UGA}^{Ser} from phage 6 and 12 to Syn61Δ3 enables infection by T4 phage We assessed the ability of T4 phage to form plaques in different strains. While T4 forms plaques in Syn61 WT, Syn61Δ3 is resistant to T4 plaque formation due to the absence of tRNAs to decode TCG and TCA codons. T4 plaque formation is rescued in Syn61Δ3 by the expression of either serT (encoding E. coli tRNA_{UGA}^{Ser}) or ΦtRNA_{UGA}^{Ser} that is encoded on the genomes of both phage 12 and phage 06. Cells were infected with ˜10⁶ to ˜10³ PFU/mL.

FIG. 29 Genetic code refactoring and code locking blocks viral replication (A) Clonal pools of phage 12 and 06 that encode ΦtRNA_{UGA}^{Ser} on their genome form plaques in both Syn61 WT and Syn61Δ3. Various strains with distinct refactored and locked genetic codes show complete ablation of plaque formation. (B) Titration (˜10⁷ to ˜10³ PFU/mL) of phage 6 and 12 on strains with the indicated genetic codes. No plaque formation was observed in strains with refactored and locked genetic codes.

FIG. 30 Phage replication is a more complex biological function than conjugation. The formation of plaques from T-4 like phage infection is a more complex biological process than the formation of colonies after successful horizontal gene transfer from conjugation. (A) Comparative genome analysis of T-4 like phages (Phage 06 and Phage 12) and the RK2 F plasmid. On the x-axis the size is indicated in kilobases. Positions where a TCA codon occurs are marked with a vertical, yellow line (top). Positions where a TCG codon occurs are marked with a vertical, red line (bottom). (B) Codon usage in mobile genetic elements. On the x-axis the total frequency of target codons (TCA and TCG) is indicated. TCA codon frequency is represented in yellow, TCG codon frequency in red. (C) For the formation of plaques a phage particle first needs to attach to a bacterial cell and inject its DNA into the cytosol. Subsequently, genes from the phage genome are transcribed and translated by the host machinery producing viral proteins. Moreover, the phage genome is replicated inside the cell. Finally, these components need to mature into fully functional phage particles that escape from the cell and infect neighbouring cells. This complex process requires many structural and regulatory proteins to be expressed correctly from the phage genome. (D) For the formation of colonies from recipient cells bearing a horizontally transferred conjugative plasmid, the donor cell first needs to attach to the recipient. Subsequently, the F plasmid is transferred through the mating channel and recircularized inside the recipient cell. The process of attachment, transfer, and recircularization relies exclusively on proteins expressed in the donor cell. Then, proteins are expressed from the conjugative element enabling its replication and segregation during cell division. Division of the recipient cell bearing the transferred plasmid leads to colony formation without further conjugation events.

FIG. 31 Code-locking ensures stability of refactored genetic codes. (A) Cells were passaged every 12 h and assessed for the presence of the refactored code. While cells with code-locking retained the genetic code in all cases cells demonstrating stability of code refactoring, in cells without code locking the refactored codes are not stable. Cells either contained a codon compressed SpecR resistance gene (not locked: −) or a cognate SpecR gene (SpecR TCG-Ala, TCA-His/SpecR TCG-Leu, TCA-Leu) (locked: +); all experiments were performed in the presence of spectinomycin. (B) Phage encoding a seryl-tRNA_{UGA} infect cells with unstable genetic codes, but not cells with stably refactored codes. Cells from the last point in the time course (A) with and without code-locking were subject to infection with T4-like phages (Phage 06/12). Plaque count indicates the number of successfully replicating phage obtained from infection with ˜5×10⁸ PFU (phage 12) and ˜1×10⁷ PFU (phage 6). Cells either contained a codon compressed SpecR resistance or a cognate SpecR gene (as in (A)); all experiments were performed in the presence of spectinomycin.

FIG. 32 Structure of major capsid protein gp23. (A) Protein structure of major capsid protein gp23 from T4 phage. There are three serine residues present that are encoded with TCA (highlighted in orange) and therefore subject to ambiguous decoding in cells with refactored genetic codes. (B) Binding interface of major capsid protein gp23; displayed are three subunits of gp23 that are part of a the hexameric capsid subunit. Serine residues encoded by TCA on the central subunit (in grey) are displayed in orange.

FIG. 33 Phage propagation assay. T4-like phage encoding a seryl-tRNA (tRNA_{UGA}^{Ser}) successfully infect Syn61Δ3 but not cells with a refactored and locked genetic code. The plaque count indicated the number of phage particles in a 7.5 uL volume after 24 h propagation in a culture containing the indicated cells.

FIG. 34 The effects of code refactoring and code locking on modes of horizontal gene transfer. T4-like phage encoding a seryl-tRNA (tRNA_{UGA}^{Ser}) successfully infect Syn61Δ3 but not cells with a refactored genetic code. Plaque count indicates the number of successfully replicating phage obtained from infection with 1.1×10¹⁰ plaque-forming units (PFU)/ml (phage 12) and 7.5×10⁹ PFU/ml (phage 6).

### DETAILED DESCRIPTION

### Code-Locking

There is a need to prevent mobile genetic elements, such as viruses, from contaminating cells. For instance, industrial scale fermentation of bacterial for commercial product production can be contaminated by mobile genetic elements, such as viruses. This can cause financial loss and can disrupt vital supply claims. There are existing methods for protecting cells from such contamination (see WO2020/229592 A1 or Robertson et al., Science, 4 Jun. 2021, Vol 372, Issue 6546, pp. 1057-1062, both incorporated herein by reference) but the inventors demonstrate herein that there remains a risk from mobile genetic elements that comprise tRNAs. Attempts have been made to reduce the risk from such mobile genetic elements (see Nyerges et al. “Swapped genetic code blocks viral infections and gene transfer”) but there remains a need for techniques to render cells resistant to mobile genetic elements that comprise tRNAs.

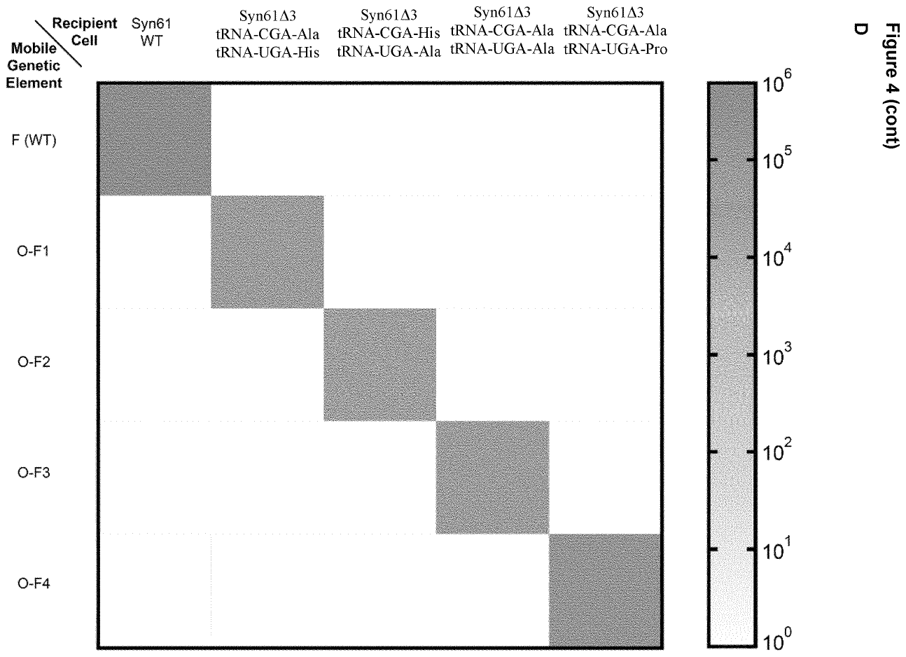

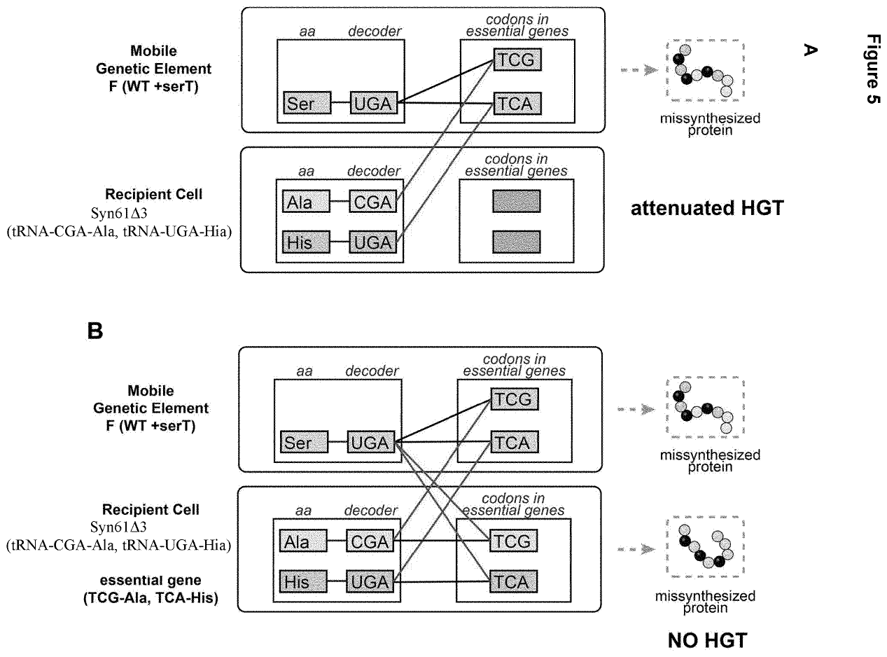

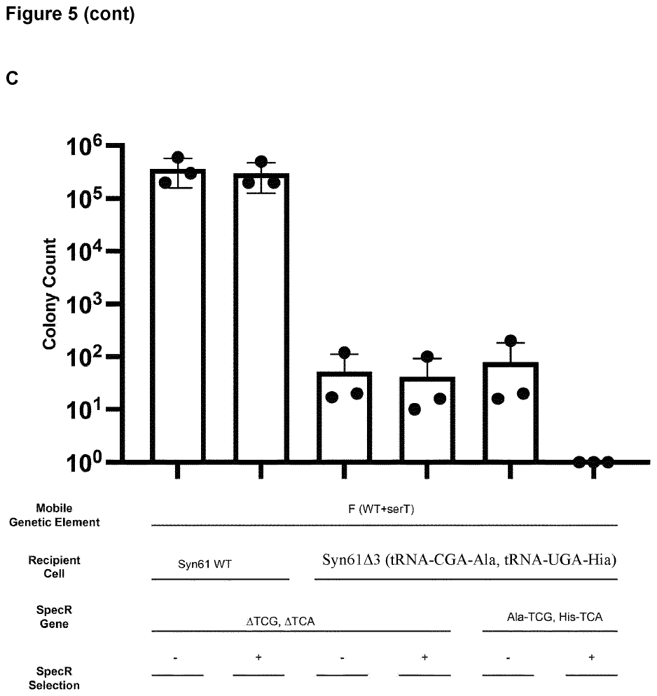

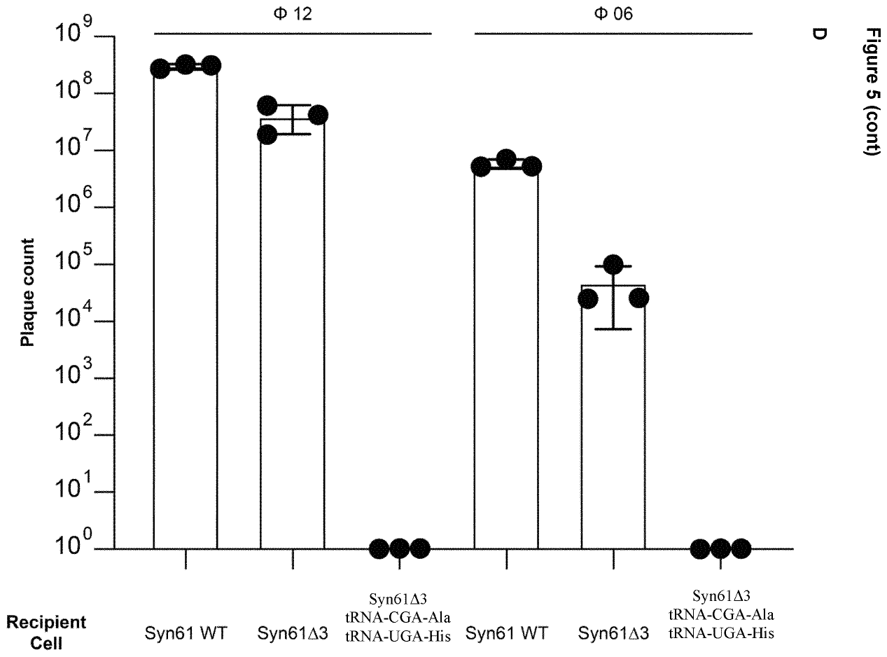

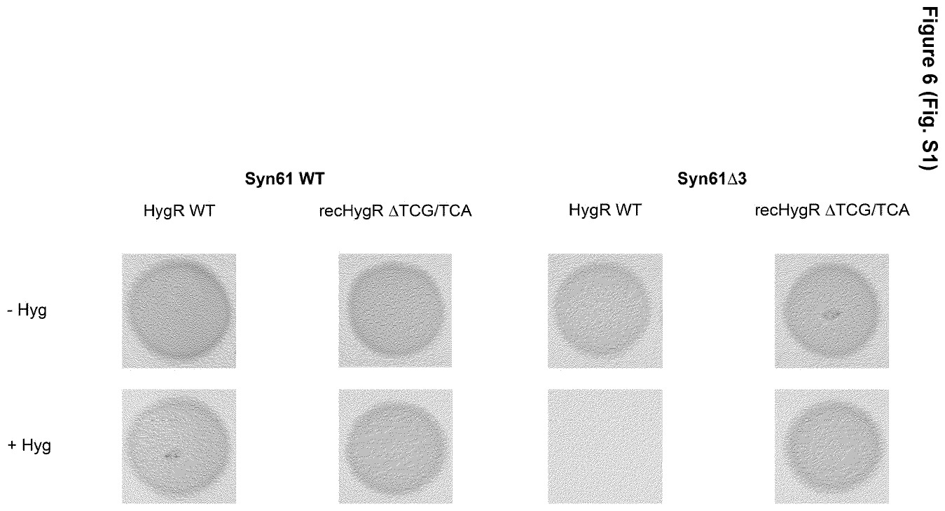

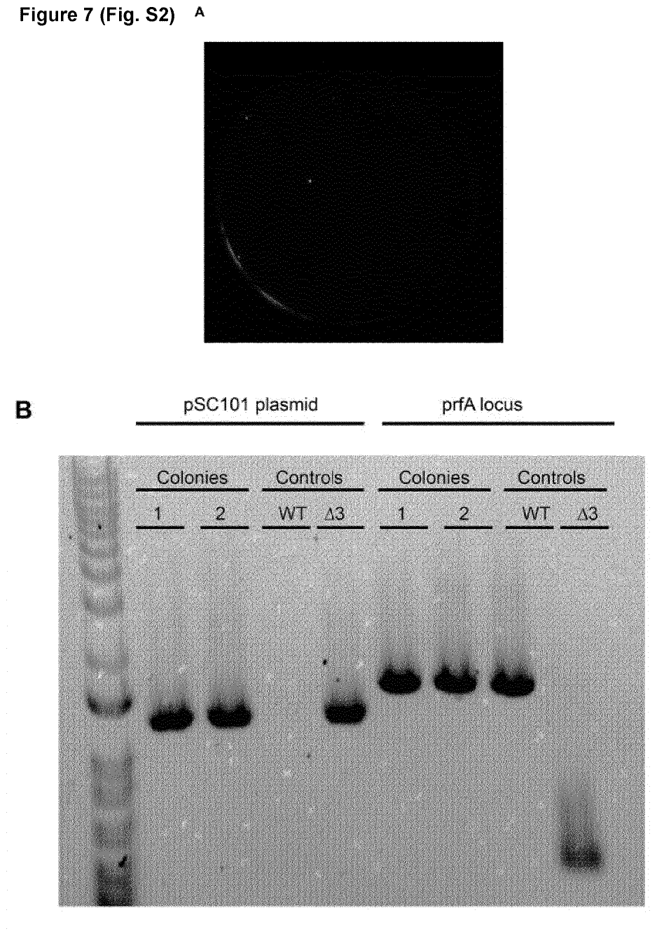

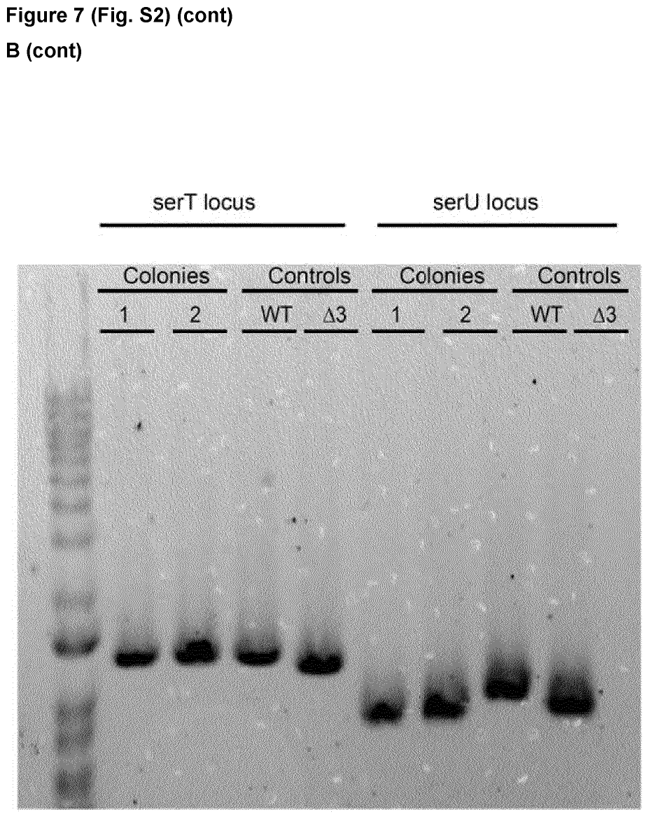

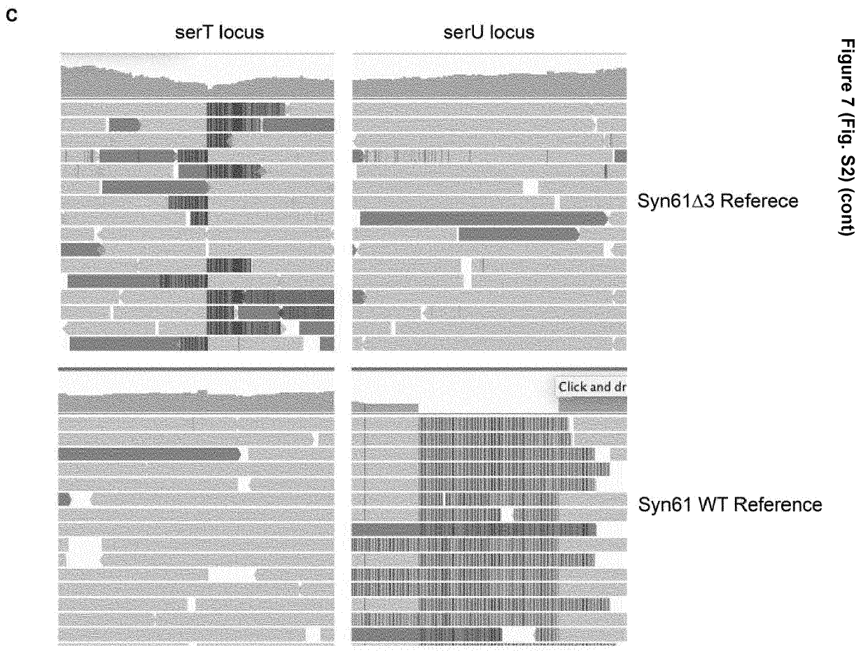

Provided herein are cells that are “code-locked”. The genome of these cells has been recoded to reduce or remove instances of at least one type of sense codon, which then allows the removal of an endogenous cognate tRNA because it is now dispensable for the cell (see Robertson et al., Science, 4 Jun. 2021, Vol 372, Issue 6546, pp. 1057-106). The inventors discovered that the inclusion of a tRNA specific for the removed sense codon, but charged with an amino acid which with the sense codon would not naturally be associated, reduces but does not ablate the risk of contamination with a mobile genetic element comprising a relevant tRNA (see FIG. 5 of the present disclosure). This has been noted by others (see Nyerges et al.). The inventors have overcome this problem by generating cells that include a gene that is required for viability of the cell, wherein the gene has been recoded so that a sense codon has been reassigned to a different amino acid from that of the canonical genetic code. The inventors have surprisingly found that the inclusion of such a gene reduces or ablates horizontal gene transfer from mobile genetic elements that make use of the canonical genetic code or a genetic code that does not match that of the target cell. The inventors have also found that this approach leads to the maintenance of exogenous tRNAs that have been introduced for code refactoring. Thus, this approach also leads to maintenance of the resistance to horizontal gene transfer or mobile genetic elements.

Thus, in a first aspect, there is provided a cell that: comprises a genome wherein at least a first type of sense codon has been recoded such that a first endogenous tRNA is dispensable; does not express the first endogenous tRNA; expresses a first modified tRNA capable of decoding the first type of sense codon, wherein the first modified tRNA is charged with a first amino acid that is not a naturally cognate amino acid for the first type of sense codon; and comprises a gene required for viability, wherein the gene comprises at least one occurrence of the first type of sense codon and the cell is viable when the first type of sense codon in said gene is decoded as the first amino acid.

The cell may have increased resistance to horizontal gene transfer or mobile genetic elements, as discussed in the section below. Hence, in a fourth aspect provided herein is a cell with increased resistance to horizontal gene transfer or mobile genetic elements, wherein the cell has been modified to reassign at least one type of sense codon to an amino acid not associated with the sense codon in the canonical genetic code, and the cell comprises a gene required for viability that is functional when decoded according to the reassigned genetic code and is not functional when decoded according to the canonical genetic code. The gene may be required for viability alone or in combination with other genes.

As discussed in Example 7, cells that have been modified in the above-mentioned manner exhibit improved maintenance of the resistance to horizontal gene transfer or mobile genetic elements. Thus, the increased resistance may be resistance that is maintained over a longer period of time compared to a cell culture that does not comprise code-locked bacteria.

The gene required for viability may be an exogenous gene. For instance, the gene may be a gene that is commonly used as a positive selectable marker. In some examples, the gene is an antibiotic resistance gene. Illustrative embodiments include a spectinomycin resistance gene or a hygromycin resistance gene.

In other examples, the gene required for viability may be an essential gene within the cell's genome. A gene is “essential”, as used herein, if the product of the gene is required for viability of the cell. For instance, if the prevention of expression of a functional form of a protein encoded by a gene would result in non-viability of the cell, then the gene is considered essential.

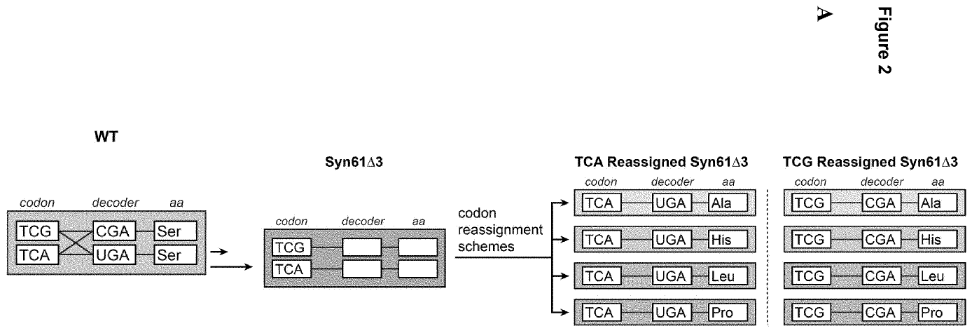

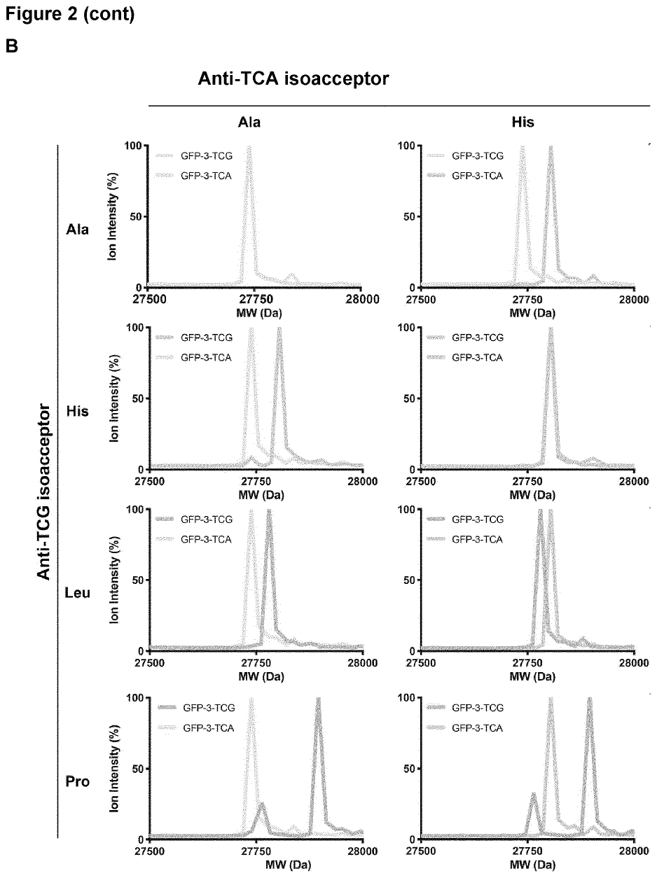

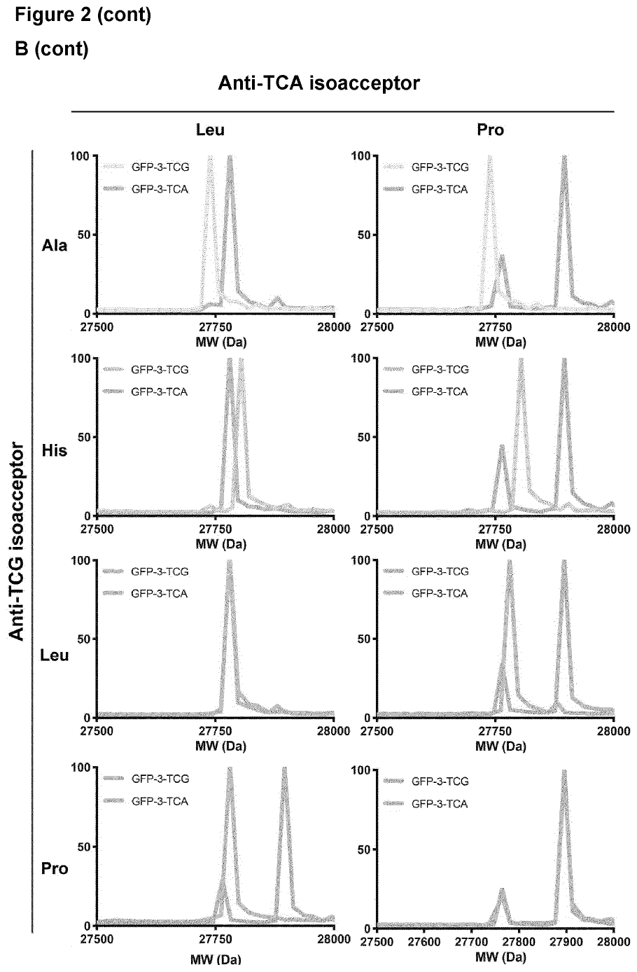

The gene required for viability may comprise at least one reassigned codon wherein a mutation of the corresponding residue in the translated product causes a loss of function. In particular, the reassigned codon may be positioned such that the decoding of the codon according to the canonical genetic code results in a loss of function for the product. For instance, if the cell comprises a tRNA capable of decoding a codon normally associated with serine but charged with alanine, and the gene comprises said codon where an alanine would be present in the natural product, the product of the gene may be one that would be non-functional with a serine in said position. The aforementioned examples of particular amino acids are purely illustrative, and any may be used. In particular, any of the reassignment schemes of FIG. 2C. The cell may therefore comprise a gene required for viability, wherein the gene comprises at least one occurrence of the first type of sense codon and the cell is not viable when the first type of sense codon is decoded according to the canonical genetic code.

The gene required for viability may comprise a plurality of reassigned codons. The plurality of reassigned codons may each, or may cumulatively, be positioned such that product comprising the non-reassigned amino acid (as discussed in the preceding paragraph) would be non-functional. The cell may therefore comprise a gene required for viability, wherein the gene comprises a plurality of occurrences of the first type of sense codon and the cell is not viable when said occurrences are decoded according to the canonical genetic code. The at least one occurrence of the first type of sense codon in the gene required for viability may at least partially contribute to a loss of viability if decoded according to the canonical genetic code, and may contribute to a complete loss of viability in combination with other features, such as other reassigned codons or other types of reassigned codon. In some examples, multiple reassigned codons, potentially of multiple types, may be present within the gene required for viability or multiple genes required for viability may be present. Any individual instance of a reassigned codon may at least partially contribute to a loss of viability if decoded according to the canonical genetic code, and the full loss of viability may be due to an effect of the translation of multiple reassigned codons according to the canonical genetic code.

The cell of the present disclosure may comprise more than one gene required for viability comprising at least one reassigned codon.

The cell of the present disclosure may comprise a genome that has been recoded with respect to a second type of sense codon.

In some embodiments, the genome of the cells is recoded such that a first endogenous tRNA is dispensable and a second endogenous tRNA is dispensable. The cell may not express or comprise the first or second endogenous tRNA. In examples, the cell expresses or comprises a second modified tRNA, which is capable of decoding the second type of sense codon. The second modified tRNA is charged with a second amino acid, and the second amino acid is not a naturally cognate amino acid for the second type of sense codon.

A gene required for viability may comprise at least one occurrence of the second type of sense codon, wherein cell is viable when the second type of sense codon in said gene is decoded as the second amino acid. This gene may be the same gene required for viability and comprising the first type of sense of codon, or may be a different gene.

In some examples, the cell is not viable when the second type of sense codon in the gene required for viability is decoded according to the canonical genetic code. The gene may comprise a plurality of occurrences of the second type of sense codon and the cell may not be viable when said occurrences are decoded according to the canonical genetic code. The at least one occurrence of the second type of sense codon in the gene required for viability may at least partially contribute to a loss of viability if decoded according to the canonical genetic code, and may contribute to a complete loss of viability in combination with other features, such as other reassigned codons or other types of reassigned codon. The full loss of viability may be due to an effect of the translation of multiple reassigned codons according to the canonical genetic code.

The cell may comprise at least one gene required for viability that comprises the first type of sense codon and at least one different gene required for viability that comprises the second type of sense codon. The cell may comprise a gene required for viability that comprises the first and the second types of sense codon. Combinations of genes required for viability, and comprising any combination of the reassigned codons, are also possible.

The cells of the present disclosure may be viable when the genes are decoded according to the reassigned genetic code and may be non-viable when the genes are decoded at least partially according to the canonical genetic code.

The modified tRNA is one that is derived from a tRNA, which may be a naturally occurring tRNA, that has been altered such that it is capable of decoding a codon to an amino acid with which the codon is not associated in the canonical genetic code. For instance, the residues of the anticodon of the tRNA may be substituted such that the tRNA has a different codon specify, such tRNAs may be referred to as anticodon-swapped tRNAs. Alternatively, it is possible to charge a tRNA with an amino acid with which it would not be naturally associated, hence providing the capability for the tRNA to decode a codon to an amino acid with which the codon is not associated in the canonical genetic code. The modified tRNAs may also be modified in other ways, for instance additional sequence may be added. The modified tRNAs may be charged with the natural amino acid with which the parent tRNAs are naturally associated.

The modified tRNA may be derived from a naturally occurring tRNA (which may be referred to as a parent tRNA). For instance, the modified tRNA may be derived from a tRNA that is endogenous to the cell in question. The modified tRNA may be derived from an isoacceptor tRNA for a particular amino acid within the cell. For instance, if the cell is E. coli the modified tRNA may be derived from an E. coli tRNA that is an isoacceptor for the first or second amino acid. The modified tRNA may be derived from a naturally occurring tRNA found in a mobile genetic element, such as a viral tRNA. The modified tRNA may comprise identity elements that are recognised by an aminoacyl-tRNA synthetase endogenous to the cell. The modified tRNA may retain the identity elements of the parent tRNA.

The inventors demonstrate herein that episomes encoding components of the translation machinery that are required for the translation of the gene-required-for-viability according to the reassigned genetic code can be essential to the cell. Hence, these episomes are stably maintained by the cells of the first aspect. As such, in an embodiment, the first modified tRNA may be encoded by an episome within the cell, such as a plasmid. The episome may further comprise other genes for which stable maintenance is desired.

In a particular example, the cell is E. coli and comprises a genome that has been recoded with respect to a first and a second type of sense codon (e.g. TCA and TCG). The first modified tRNA may be an E. coli isoacceptor tRNA for a first amino acid (e.g. alanine) that has been altered to comprise an anticodon complementary to the first type of sense codon (e.g. TCA). The second modified tRNA may be an E. coli isoacceptor tRNA for a second amino acid (e.g. histidine) that has been altered to comprise an anticodon complementary to the first type of sense codon (e.g. TCG).

In some examples, the first modified tRNA cannot decode the second type of sense codon and/or the second modified tRNA cannot decode the first type of sense codon. In further examples, the first modified tRNA cannot decode any type of codon apart from the first type of sense codon and/or the second modified tRNA cannot decode any type of codon apart from the second type of sense codon.

The first and the second amino acids may be the same amino acid, for instance they may both be alanine, histidine, leucine, or proline. In other examples, the first and second amino acids may be different. For example, one may be alanine where the other is leucine, etc. Some exemplary reassignment schemes are shown in FIG. 2C.

The cell may be described as comprising tRNA_{XXX}^{Xaa} (where XXX is the type of sense codon and Xaa is the charged amino acid). Thus, a cell making use of a particular reassignment scheme may be defined as comprising the relevant tRNA. For instance, in particular embodiments, a cell making use of a reassignment of TCG to alanine and TCA to histidine comprises tRNA_{CGA}^{Ala} and tRNA_{UGA}^{His}; and a cell making use of a reassignment of TCG to histidine and TCA to alanine comprises tRNA_{CGA}^{His} and tRNA_{UGA}^{Ala}.

The first and/or second amino acid may be a naturally occurring amino acid. The naturally occurring amino acid may be any natural proteinogenic amino acid. A “natural proteinogenic amino acid” is any one of L-alanine, L-cysteine, L-aspartic acid, L-glutamic acid, L-phenylalanine, glycine, L-histidine, L-isoleucine, L-lysine, L-leucine, L-methionine, L-asparagine, L-proline, L-glutamine, L-arginine, L-serine, L-threonine, L-valine, L-tryptophan and L-tyrosine, L-pyrrolysine, and L-selenocysteine. The naturally occurring amino acid may be a canonical amino acid. A “canonical amino acid” is any one of L-alanine, L-cysteine, L-aspartic acid, L-glutamic acid, L-phenylalanine, glycine, L-histidine, L-isoleucine, L-lysine, L-leucine, L-methionine, L-asparagine, L-proline, L-glutamine, L-arginine, L-serine, L-threonine, L-valine, L-tryptophan, and L-tyrosine.

The cell of the first aspect may be any species or type as disclosed herein. For instance, the cell may be a bacterial cell with a genome recoded with regards to codons TCA and TCG, which lacks tRNA^{Ser}_{UGA} and tRNA^{Ser}_{CGA}, and wherein TCA and TCG have been reassigned. The genome of the cells may have been recoded in any manner as discussed herein. The reassignment scheme for the cells of the first aspect may be any disclosed herein, for instance one of the schemes illustrated in FIG. 2C.

The cell may be Syn61, a strain that is derived from Syn61, or recoded in the same manner as Syn61. The cell may be Syn61Δ3, a strain that is derived from Syn61Δ3, or may be modified in the same manner as Syn61Δ3.

The features of the first aspect, which relate to “code locking”, may be applied to the recoding schemes of the second aspect or the third aspect. Thus, any features of the first, second, and third aspects may be combined and are not mutually exclusive. The features of the second and third aspect, for instance the tRNAs and the coding schemes, may be applied to the first aspect.

The cells of the first aspect may have increased resistance to mobile genetic elements. The cells of the first aspect may have improved maintenance of resistance to mobile genetic elements; for instance the resistance may be maintained in a cell culture for a longer period when compared to a control culture not comprising code-locked cells.

### Orthogonal Coding Schemes

There are an increasing number of applications for genetically modified organisms, and a need to limit the transfer of genetic information from these organisms to natural organisms. The inventors provide herein orthogonal coding schemes, which prevent the transfer of genetic information to natural organisms or to organisms making use of alternative orthogonal coding schemes. For instance, mobile genetic elements that make use of one of said orthogonal coding schemes cannot transfer to or be expressed by a natural organism.

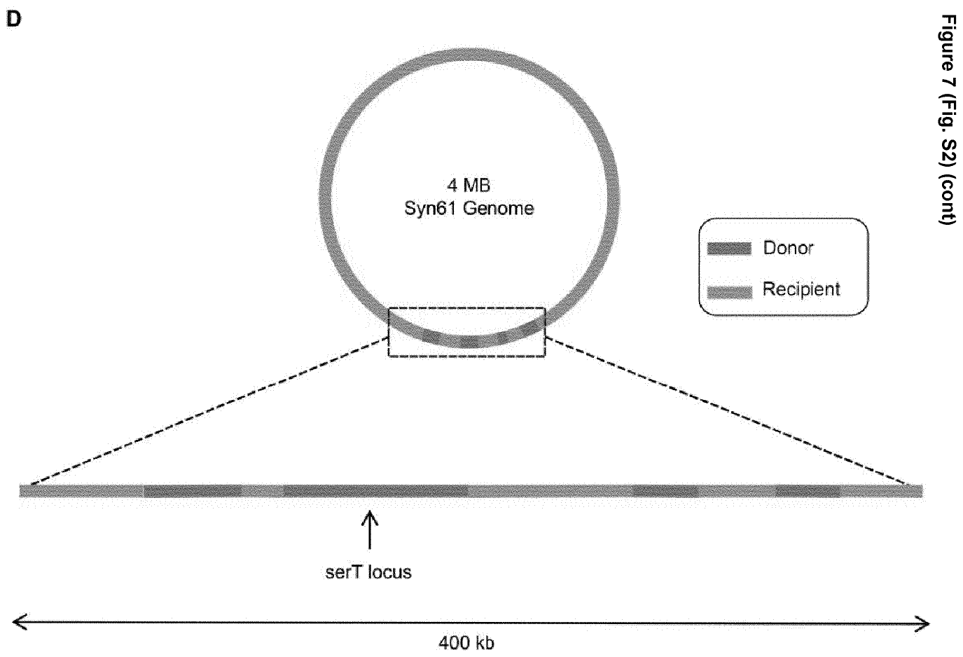

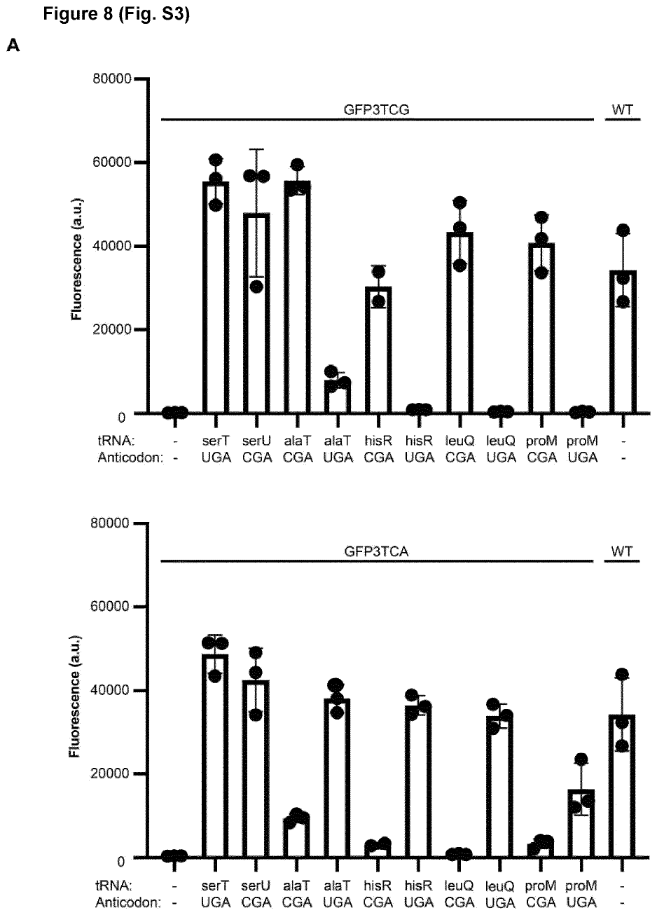

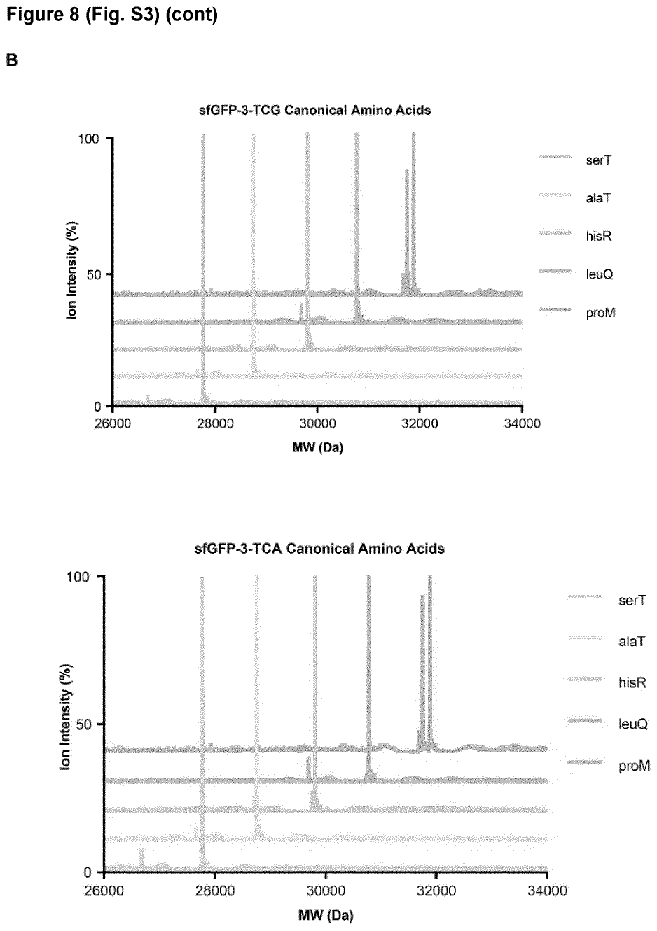

Others have attempted to generate synthetic genetic information to prevent horizontal gene transfer (Nyerges et al. “Swapped genetic code blocks viral infections and gene transfer”). However, the inventors provide herein a screening method that allows the development of tRNAs that are active and specific (see FIG. 8 (FIG. S3). As the skilled person would appreciate, the screen, as disclosed in the examples, can be adapted for use with codons other than TCA and TCG and enables the development of active and specific tRNAs that do not decode off-target codons.

Thus, in a second aspect, there is provided a cell that: comprises a genome wherein a first type of sense codon and a second type of sense codon have been recoded such that a first endogenous tRNA and a second endogenous tRNA are dispensable; does not express the first endogenous tRNA and the second endogenous tRNA; expresses a first anticodon-swapped tRNA derived from a naturally occurring first parent tRNA, wherein the first anticodon-swapped tRNA is charged with a first amino acid and the first parent tRNA is an isoacceptor for the first amino acid, and wherein the first amino acid is not a naturally cognate amino acid for the first type of sense codon; and expresses a second anticodon-swapped tRNA derived from a naturally occurring second parent tRNA, wherein the second anticodon-swapped tRNA is charged with a second amino acid and the second parent tRNA is an isoacceptor for the second amino acid, and wherein the second amino acid is not a naturally cognate amino acid for the second type of sense codon; wherein the first and/or second modified tRNA cannot decode any type of codon apart from the first type of sense codon and/or the second type of sense codon.

In a particular embodiment, a tRNA does not decode a particular codon when the rate of misincorporation is undetectable in a screening method disclosed herein or is too low to affect the fitness of the cell. Thus, the tRNAs of the second aspect may not have a detectable rate of misincorporation of non-target codons, or may not have a rate of misincorporation that would be relevant considering the size of the host genome in question. In an embodiment, the tRNAs of the second aspect are as active and specific as the tRNAs exemplified in Example 3, namely any one of tRNA_{CGA}^{Ala}, tRNA_{UGA}^{Ala}, tRNA_{CGA}^{His}, tRNA_{UGA}^{His}, tRNA_{CGA}^{Leu}, tRNA_{UGA}^{Leu}, and tRNA_{CGA}^{Leu}, tRNA_{UGA}^{Leu}.

An anticodon-swapped tRNA is one where the residues of the anticodon have been substituted such that the tRNA has a different codon specificity. The anticodon-swapped tRNAs may also be modified in other ways, for instance additional sequence may be added.

The present inventors have surprisingly found that sense codons which canonically encode the same amino acid, and which would canonically be decoded by the same tRNA or overlapping tRNAs due to wobble base pairing, may be used to code for multiple alternative amino acids. It would have been expected that such sense codons would only allow for a single reassignment. The inventors provide herein a screening method that enables the development of tRNAs with the required activity and specificity. This finding is advantageous because in an exemplary organism with, for example, two reassigned serine codons, the inventors are able to generate many different orthogonal codes. As an illustration, the inventors have generated 16 refactored codes from just two reassigned sense codons and four amino acids.

Thus, in some examples, the first and second type of sense codon would canonically be decoded by the same tRNA or overlapping tRNAs due to wobble base pairing. The first anticodon-swapped tRNA may be unable to decode any codon type apart from the first type of sense codon and the second anticodon-swapped tRNA may be unable to decode any codon type apart from the second type of sense codon. This allows the first and second types of sense codon to be used to code for two different amino acids, without misincorporation.

The first type of sense codon and the second type of sense codon may be of the formula XXN. This means that the first and the second bases are the same, whereas the third base is different. In examples, the first anticodon-swapped tRNA cannot decode the second type of sense codon and the second anticodon-swapped tRNA cannot decode the first type of sense codon.

In some examples, the first anticodon-swapped tRNA cannot decode any codon type apart from the first type of sense codon and/or the second anticodon-swapped tRNA cannot decode any codon type apart from the second type of sense codon.

In particular embodiments, the first anticodon-swapped tRNA does not decode TCC or TCT codons and the second anticodon-swapped tRNA does not decode TCC or TCT codons. As an example, this may be advantageous when the first or second type of recoded sense codon is TCA or TCG because mis-incorporation at TCC or TCT codons may reduce the fitness of the cell. For instance, some E. coli genomes comprise 9,999 TCC codons and 9,566 TCT codons, and so misincorporation can affect fitness. In a particular embodiment, a tRNA does not decode a particular codon when the rate of misincorporation is undetectable in a screening method disclosed herein or too low to affect the fitness of the cell. Thus, the tRNAs may not have a detectable rate of misincorporation at TCC or TCT, or may not have a rate of misincorporation that would be relevant considering the size of the host genome in question. In an embodiment, the tRNAs of have a rate of misincorporation at TCC or TCT that is no higher than any one of tRNA_{CGA}^{Ala}, tRNA_{UGA}^{Ala}, tRNA_{CGA}^{His}, tRNA_{UGA}^{His}, tRNA_{CGA}^{Leu}, tRNA_{UGA}^{Leu}, and tRNA_{CGA}^{Leu}, tRNA_{UGA}^{Leu} as exemplified in Example 3.

The anticodon-swapped tRNAs of the second aspect are charged with the natural amino acid with which they are naturally associated. Thus, the anticodon-swapped tRNAs are charged with the same amino acid as the parent tRNA from which they are derived. The anticodon-swapped tRNA may comprise identity elements that are recognised by an aminoacyl-tRNA synthetase endogenous to the cell.

The anticodon-swapped tRNA may be derived from a tRNA that is endogenous to the cell in question. The anticodon-swapped tRNA may be derived from an isoacceptor tRNA for a particular amino acid within the cell. For instance, if the cell is E. coli the anticodon-swapped tRNA may be derived from an E. coli tRNA that is an isoacceptor for the first or second amino acid. The anticodon-swapped tRNA may be derived from a naturally occurring tRNA found in a mobile genetic element, such as a viral tRNA. The anticodon-swapped tRNA may retain the identity elements of the parent tRNA.

In examples, the first and second type of sense codon may both canonically encode serine, may both canonically encode alanine, or may both canonically encode leucine.

In a particular embodiment, the first type of sense codon is TCA and the second type of sense codon is TCG.

The first and/or second type of sense codon may be reassigned to any natural proteinogenic amino acid or canonical amino acid. In illustrative embodiments, the first type of sense codon, for instance a canonical serine codon, may be reassigned to one of alanine, histidine, leucine, and proline, and the second type of sense codon, for instance a canonical serine codon, may be reassigned to one of alanine, histidine, leucine, and proline.

In particular examples, TCA may be reassigned to any non-serine natural proteinogenic amino acid or canonical amino acid and/or TCG may be reassigned to any non-serine natural proteinogenic amino acid or canonical amino acid. In further illustrative embodiments, TCA may be reassigned to one of alanine, histidine, leucine, and proline, and TCG may be reassigned to one of alanine, histidine, leucine, and proline. In some examples, the reassignment scheme is as disclosed in FIG. 2C.

In other examples, the first amino acid and the second amino acid are different types of amino acid. The following are exemplary reassignment schemes:
TCG to alanine and TCA to histidine
TCG to alanine and TCA to leucine
TCG to alanine and TCA to proline
TCG to histidine and TCA to alanine
TCG to histidine and TCA to leucine
TCG to histidine and TCA to proline
TCG to leucine and TCA to alanine
TCG to leucine and TCA to histidine
TCG to leucine and TCA to proline
TCG to proline and TCA to alanine
TCG to proline and TCA to histidine
TCG to proline and TCA to leucine.

The first or second anticodon-swapped tRNA may derived from a parent tRNA encoded by ArgQ, ArgU, GltU, HisR, ProK, ProL, ProM, TrpT, ThrU, ThrT, TyrU, TyrV, AlaT, or LeuQ. Hence, the first or second anticodon-swapped tRNA may be encoded by any one said of genes, wherein the anticodon has been modified such that the tRNA recognises a type of sense codon that is not canonically associated with the amino acid with which the parent tRNA is charged. In a particular example, the first or second anticodon-swapped tRNA may be derived from a parent tRNA encoded by HisR, ProM, AlaT, or LeuQ. The genes encoding the tRNA may be derived from E. coli.

In some examples, the first and the second anticodon-swapped tRNAs are derived from a parent tRNA encoded by the group consisting of: ArgQ, ArgU, GltU, HisR, ProK, ProL, ProM, TrpT, ThrU, ThrT, TyrU, TyrV, AlaT, and LeuQ. In some examples, the first and the second anticodon-swapped tRNAs are derived from a parent tRNA encoded by the group consisting of: HisR, ProM, AlaT, and LeuQ.

In some examples the ArgQ, ArgU, GltU, HisR, ProK, ProL, ProM, TrpT, ThrU, ThrT, TyrU, TyrV, AlaT, or LeuQ gene is unmodified except the anticodon. In other examples, the gene may include additional sequence or be truncated. In particular examples, the encoded tRNA may comprise the identity elements of the parent tRNA. In other examples, the genes may comprise one or more modifications, wherein the encoded tRNA remains functional. The genes may comprise 10 or fewer, 9 or fewer, 8 or fewer, 7 or fewer, 6 or fewer, 5 or fewer, 4 or fewer, 3 or fewer, 2 or fewer, 1, or no substitutions, additions, or replacements.

In some examples, the anticodon is swapped to UGA or CGA. In some examples, the first anticodon-swapped tRNA is swapped to UGA and the second anticodon-swapped tRNA is swapped to CGA.

In particular examples, the ArgQ-derived tRNA is according to SEQ ID NO: 43 or 44, the GltU-derived tRNA is according to SEQ ID NO: 19 or 20, the HisR-derived tRNA is according to SEQ ID NO: 49 or 50, the ProK-derived tRNA is according to SEQ ID NO: 55 or 56, the ProL-derived tRNA is according to SEQ ID NO: 57 or 58, the ProM-derived tRNA is according to SEQ ID NO: 59 or 60, the TrpT-derived tRNA is according to SEQ ID NO: 61 or 62, the ThrU-derived tRNA is according to SEQ ID NO: 25 or 26, the ThrT-derived tRNA is according to SEQ ID NO: 23 or 24, the TyrV-derived tRNA is according to SEQ ID NO: 63 or 64, the AlaT-derived tRNA is according to SEQ ID NO: 65 or 66, and the LeuQ-derived tRNA is according to SEQ ID NO: 67 or 68. Any of these sequences may comprise one or more modifications, wherein the encoded tRNA remains functional. Any of these sequences may comprise 10 or fewer, 9 or fewer, 8 or fewer, 7 or fewer, 6 or fewer, 5 or fewer, 4 or fewer, 3 or fewer, 2 or fewer, 1, or no substitutions, additions, or replacements. The modifications may be in a region not encoding an identity element. Any of these sequences may be modified to encode a different anticodon. The alternative anticodon may not be the naturally associated anticodon.

The cell of the second aspect may be any species or type as disclosed herein. For instance, the cell may be a bacterial cell with a genome recoded with regards to codons TCA and TCG, which lacks tRNA^{Ser}_{UGA} and tRNA^{Ser}_{CGA}. The genome of the cells may have been recoded in any manner as discussed herein. The cell may be Syn61, a strain that is derived from Syn61, or recoded in the same manner as Syn61. The cell may be Syn61Δ3, a strain that is derived from Syn61Δ3, or may be modified in the same manner as Syn61Δ3.

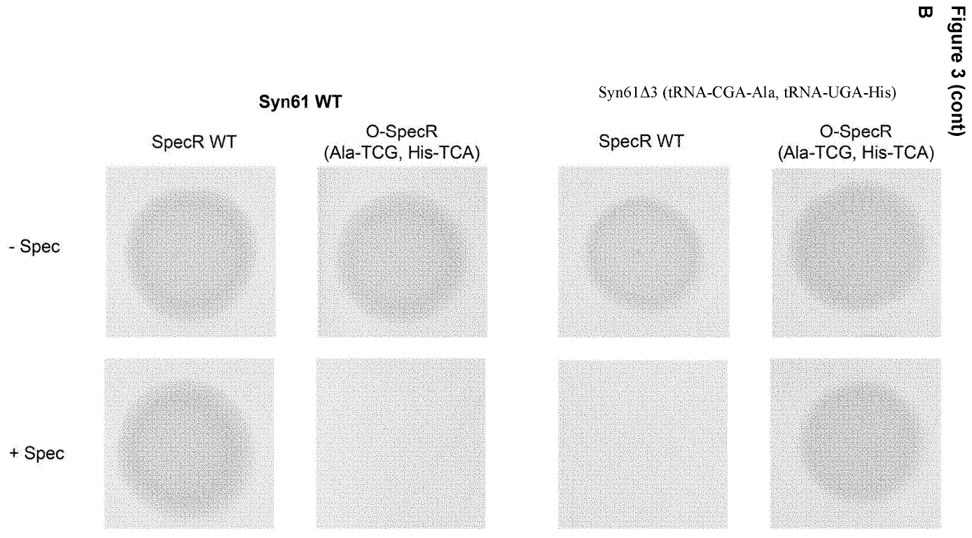

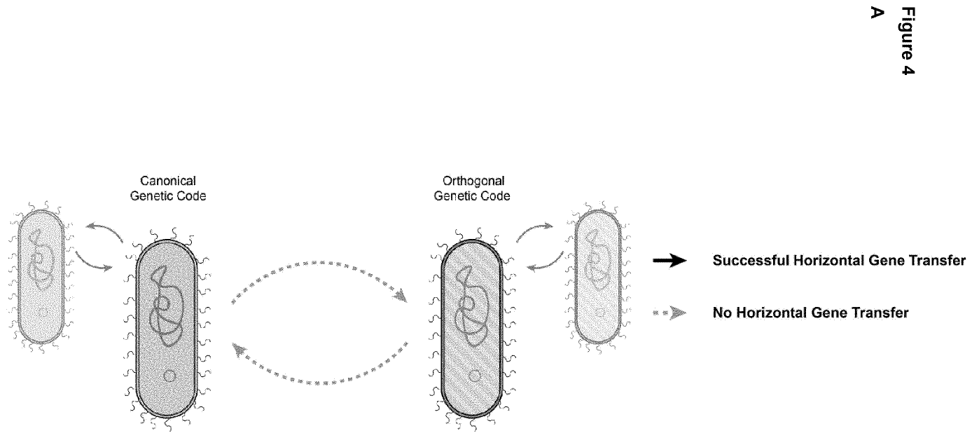

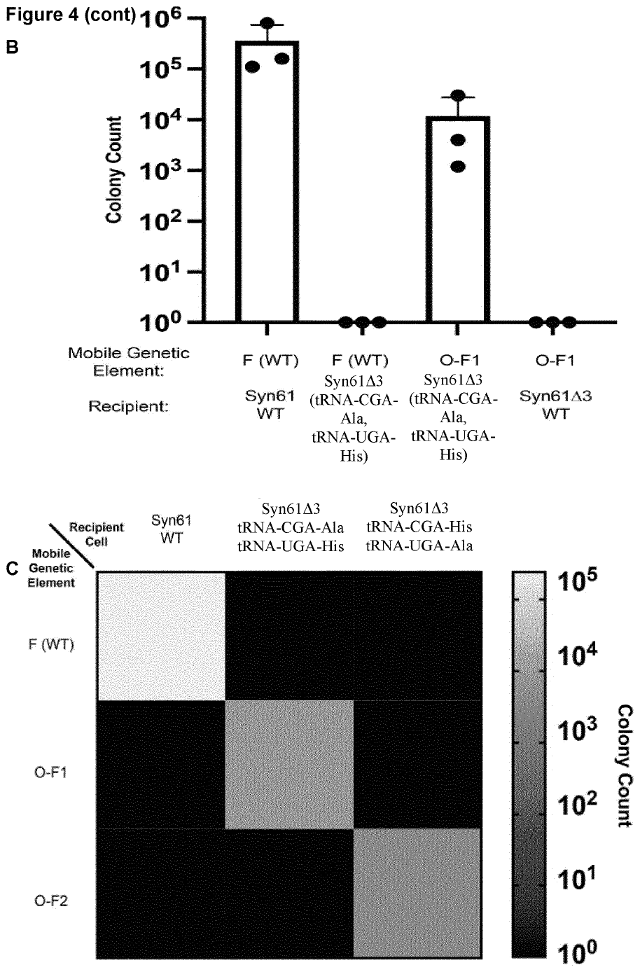

Reassignment schemes may vary in their efficiency. For instance, FIG. 4C compares two different genetic coding schemes and notes differences in the colony counts. The reassignment scheme can affect the fitness of the cell. The contribution of verified reassignment schemes is therefore a valuable contribution.

Thus, in a third aspect, there is provided a cell that: comprises a genome wherein a first type of sense codon and a second type of sense codon have been recoded such that a first endogenous tRNA and a second endogenous tRNA are dispensable; does not express the first endogenous tRNA and the second endogenous tRNA; expresses a first modified tRNA capable of decoding the first type of sense codon, wherein the first modified tRNA is charged with a first amino acid that is not a naturally cognate amino acid for the first type of sense codon; and expresses a second modified tRNA capable of decoding the second type of sense codon, wherein the second modified tRNA is charged with a second amino acid that is not a naturally cognate amino acid for the second type of sense codon; wherein: i) the first amino acid is alanine and the second amino acid is alanine; ii) the first amino acid is alanine and the second amino acid is histidine; iii) the first amino acid is alanine and the second amino acid is leucine; iv) the first amino acid is alanine and the second amino acid is proline; v) the first amino acid is histidine and the second amino acid is alanine; vi) the first amino acid is histidine and the second amino acid is histidine; vii) the first amino acid is histidine and the second amino acid is leucine; viii) the first amino acid is histidine and the second amino acid is proline; ix) the first amino acid is leucine and the second amino acid is alanine; x) the first amino acid is leucine and the second amino acid is histidine; xi) the first amino acid is leucine and the second amino acid is proline; xii) the first amino acid is proline and the second amino acid is alanine; xiii) the first amino acid is proline and the second amino acid is histidine; xiv) the first amino acid is proline and the second amino acid is leucine; or xv) the first amino acid is proline and the second amino acid is proline.

The modified tRNA may be as discussed for the first or second aspect. In particular, the first modified tRNA may be unable to decode the second type of sense codon and/or the second modified tRNA may be unable to decode the first type of sense codon. In some examples, the first modified tRNA cannot decode any type of codon apart from the first type of sense codon and/or the second modified tRNA cannot decode any type of codon apart from the second type of sense codon. This high specificity is enabled for the first time by the screening methods disclosed herein.

The modified tRNA is one that is derived from a tRNA, which may be a naturally occurring tRNA, that has been altered such that it is capable of decoding a codon to an amino acid with which the codon is not associated in the canonical genetic code. For instance, the residues of the anticodon of the tRNA may be substituted such that the tRNA has a different codon specify, such tRNAs may be referred to as anticodon-swapped tRNAs. The modified tRNAs may also be modified in other ways, for instance additional sequence may be added. The modified tRNAs may be charged with the natural amino acid with which the parent tRNAs are naturally associated. The modified tRNA may be derived from a naturally occurring tRNA (which may be referred to as a parent tRNA). For instance, the modified tRNA may be derived from a tRNA that is endogenous to the cell in question. The modified tRNA may be derived from an isoacceptor tRNA for a particular amino acid within the cell. For instance, if the cell is E. coli the modified tRNA may be derived from an E. coli tRNA that is an isoacceptor for the first or second amino acid. The modified tRNA may be derived from a naturally occurring tRNA found in a mobile genetic element, such as a viral tRNA. The modified tRNA may comprise identity elements that are recognised by an aminoacyl-tRNA synthetase endogenous to the cell. The modified tRNA may retain the identity elements of the parent tRNA.

The recoding scheme may be any as discussed herein. In a particular embodiment, the first type of sense codon is TCA and the second type of sense codon is TCG.

The cell of the third aspect may be any species or type as disclosed herein. For instance, the cell may be a bacterial cell with a genome recoded with regards to codons TCA and TCG, which lacks tRNA^{Ser}_{UGA} and tRNA^{Ser}_{CGA}. The cell may be Syn61, a strain that is derived from Syn61, or recoded in the same manner as Syn61. The cell may be Syn61Δ3, a strain that is derived from Syn61Δ3, or may be modified in the same manner as Syn61Δ3.

### Kits Comprising Cells Making Use of Mutually Orthogonal Coding Schemes

The inventors have discovered that cells making use of a first orthogonal code may be mutually orthogonal with cells making use of a second orthogonal code (see FIG. 4C). Thus, such cells may coexist with each other, and optionally with cells making use of the canonical genetic code, and no horizontal gene transfer will be able to take place between cells not of the same coding scheme.

Thus, in a sixth aspect of the invention, there is provided a kit comprising a first cell recoded according to a first orthogonal coding scheme and a second cell recoded according to a second orthogonal coding scheme, where the first and second coding schemes are mutually orthogonal.

In an example, the kit comprises a first cell of the first, second, or third aspect and a second cell of the first, second, or third aspect, wherein the first and second cells make use of coding schemes that are mutually orthogonal.

In some examples, the first and/or second orthogonal genetic coding scheme is any disclosed herein, such as any orthogonal genetic code of FIG. 2C.

In an example, the kit may comprise a first cell, which may be a bacterial cell such as E. coli, that makes use of a reassignment scheme illustrated in FIG. 2C. The second cell of the kit, which may be a bacterial cell such as E. coli, may make use of a different reassignment scheme illustrated in FIG. 2C. In some examples, the cells are based upon or derived from Syn61.

In a particular example, the first cell comprises tRNA_{CGA}^{Ala} and tRNA_{UGA}^{His} or comprises tRNA_{CGA}^{His} and tRNA_{UGA}^{Ala}.

The kit may further comprise a cell that makes use of the canonical genetic code.

The kit may further comprise a first mobile genetic element that has been recoded according to the first orthogonal coding scheme. The kit may further comprise a second mobile genetic element that has been recoded according to the second orthogonal coding scheme. The first and/or the second mobile genetic element may be a mobile genetic element as disclosed herein.

In an example, the kit may comprise a mobile genetic element where at least one, a plurality, or every instance of a codon canonically encoding alanine (i.e. the GCN codons) in at least one gene required for horizontal transfer of genetic information has been replaced with TCG or TCA, and the kit may comprise a cell expressing a modified tRNA capable of decoding TCG or TCA to alanine. The tRNA may be as disclosed for the first, second, or third aspect.

In an example, the kit may comprise a mobile genetic element where at least one, a plurality, or every instance of a codon canonically encoding histidine (i.e. the CAT/C codons) in at least one gene required for horizontal transfer of genetic information has been replaced with TCG or TCA, and may comprise a cell expressing a modified tRNA capable of decoding TCG or TCA to histidine. The tRNA may be as disclosed for the first, second, or third aspect.

In a particular example, the kit may comprise a mobile genetic element where at least one, a plurality, or every instance of the GCN codons in at least one gene required for horizontal transfer of genetic information have been replaced with TCG codons; and at least one, a plurality, or every instance of the CAT/C codons in at least one gene required for horizontal transfer of genetic information have been replaced with TCA codons.

The kit may further comprise a third, fourth, fifth, or further cell, wherein each of the third, fourth, fifth, or further cell makes use of a coding scheme that is mutually orthogonal with every other cell.

### Mobile Genetic Elements

The inventors demonstrate that mobile genetic elements that make use of an orthogonal genetic code are unable to transfer to cells making use of the canonical genetic code or to cells making use of a mutually orthogonal genetic code. It is shown that horizontal gene transfer is prevented in mobile genetic elements, for instance F plasmids that transfer via conjugation. This is an improvement over orthogonal genetic elements that would need to be electroporated, as such elements are not truly mobile.

Thus, in a seventh aspect, there is provided a mobile genetic element recoded according to an orthogonal coding scheme.

The orthogonal coding scheme may be any as discussed herein, including any in FIG. 2C.

In an embodiment, there is provided a mobile genetic element where at least one, a plurality, or every instance of a particular type of sense codon in at least one gene required for horizontal transfer of genetic information is replaced with a sense codon that canonically encodes a different amino acid.

In an embodiment, there is provided a mobile genetic element where at least one, a plurality, or every instance of a codon canonically encoding alanine, leucine, histidine, proline, or any combination thereof, in at least one gene required for horizontal transfer of genetic information is replaced with a sense codon that does not encode the respective amino acid. In some examples, the new sense codon may canonically encode serine, such as TCA or TCG.

In an embodiment, there is provided a mobile genetic element where at least one, a plurality, or every instance of a codon canonically encoding alanine, leucine, histidine, proline, or any combination thereof, in at least one gene required for horizontal transfer of genetic information is replaced with TCG or TCA.

For instance, at least one, a plurality, or every occurrence of the codons canonically encoding alanine (the GCN codons) in at least one gene required for horizontal transfer of genetic information may be replaced in the mobile genetic element with a codon that has been reassigned to alanine. In an embodiment, there is provided a mobile genetic element where at least one, a plurality, or every instance of a codon canonically encoding alanine (i.e. the GCN codons) in at least one gene required for horizontal transfer of genetic information has been replaced with TCG or TCA.

Alternatively or in addition, at least one, a plurality, or every instance of the codons canonically encoding histidine (the CAT/C codons) in at least one gene required for horizontal transfer of genetic information may be replaced with a codon that has been reassigned to histidine. In an embodiment, there is provided a mobile genetic element where at least one, a plurality, or every instance of a codon canonically encoding histidine (i.e. the CAT/C codons) in at least one gene required for horizontal transfer of genetic information has been replaced with TCG or TCA.

In an embodiment, there is provided a mobile genetic element where at least one, a plurality, or every instance of a codon canonically encoding alanine (the GCN codons) in at least one gene required for horizontal transfer of genetic information has been replaced with TCG and at least one, a plurality, or every instance of a codon canonically encoding histidine (the CAT/C codons) in at least one gene required for horizontal transfer of genetic information has been replaced with TCA.

The mobile genetic element includes at least one gene required for horizontal transfer of genetic information that has been recoded according to a reassignment scheme. The mobile genetic element may comprise two, three, four, or more such genes. Genes within the mobile genetic element that are not required for the horizontal transfer of genetic information may be recoded to have a compressed coding scheme, e.g. one or more type of sense codon may not be present in said gene. The genes within the mobile genetic element that are not required for the horizontal transfer of genetic information may also comprise one or more codons that have been reassigned (e.g. replaced with another codon according to a reassignment scheme). Thus, in some embodiments, all of the genes in the mobile genetic element have been recoded such that one or more type of sense codon is not present and one or more of the genes, and the mobile gene element comprises at least one gene required for horizontal transfer of genetic information that has been recoded according to a reassignment scheme.

In some examples, the mobile genetic element may be a plasmid or a virus. The mobile genetic element may be a phage. The mobile genetic element may be the F plasmid.

In one aspect of the invention, there is provided a kit comprising a first mobile genetic element as disclosed herein and a first cell of the first, second, or third aspect as disclosed herein, wherein the first mobile genetic element and the first cell make use of the same genetic coding scheme.

In an example, the kit may comprise a mobile genetic element where at least one, a plurality, or every instance of a codon canonically encoding alanine (the GCN codons) in at least one gene required for horizontal transfer of genetic information has been replaced with TCG or TCA, and may comprise a cell expressing a modified tRNA capable of decoding TCG or TCA to alanine. The tRNA may be as disclosed for the first, second, or third aspect. Alternatively, or in addition, the kit may comprise a mobile genetic element where at least one, a plurality, or every instance of a codon canonically encoding histidine (the CAT/C codons) in at least one gene required for horizontal transfer of genetic information has been replaced with TCG or TCA, and may comprise a cell expressing at modified tRNA capable of decoding TCG or TCA to histidine In other examples the kit may comprise a mobile genetic element and a cell that make use of any orthogonal genetic code disclosed herein. For instance, any orthogonal genetic code of FIG. 2C.

The kit may comprise a second mobile genetic element as disclosed herein and a second cell of the of the first, second, or third aspect, wherein the second mobile genetic element and the second cell make use of the same genetic coding scheme, and wherein the first mobile genetic element and the second mobile genetic element make use of different genetic coding schemes. In some examples, the genetic coding scheme of the second mobile genetic element and the second cell is any disclosed herein, such as any orthogonal genetic code of FIG. 2C.

The kit may further comprise third, fourth, fifth, or further mobile genetic elements and cells, wherein each pair of mobile genetic element and cell is compatible and orthogonal with every other pair.

### Methods of Increasing the Resistance of a Cell to Mobile Genetic Elements or Horizontal Gene Transfer

In a fifth aspect of the invention, there is provided a method of increasing the resistance of a cell to mobile genetic elements or horizontal gene transfer, wherein the cell has been modified to reassign at least one type of sense codon to an amino acid not associated with the sense codon in the canonical genetic code, said method comprising: modifying a gene required for viability to include at least one occurrence of the reassigned sense codon, wherein the cell is viable if the reassigned sense codon in said gene is decoded as the reassigned amino acid, and the cell is not viable if the reassigned sense codon in said gene is decoded according to the canonical genetic code, or wherein the reassigned sense codon in said gene at least partially contributes to a loss of viability if decoded according to the canonical genetic code.

The increased resistance may be resistance that is maintained for a longer period of time. For instance, resistance that is not lost during prolonged cell culture (see Example 7). Thus, the cells of the fifth aspect may exhibit resistance to horizontal gene transfer or mobile genetic elements that is maintained over a longer period of time compared to cells that are not code locked.

The genome of the cell may have been recoded to remove instances of at least one type of sense codon. The recoding may be any as disclosed herein, for instance to recode TCA or TCG. The cell may not express at least one endogenous tRNA, such as tRNA^{Ser}_{UGA} or tRNA^{Ser}_{CGA}. The assignment may be due to the insertion of a modified tRNA or modified tRNAs. The modified tRNA may be any as disclosed herein, for instance anticodon-swapped isoacceptor tRNAs for any of alanine, leucine, histidine, or proline.

The gene required for viability may be any, including any disclosed herein. For instance, the gene may be an essential gene or a positive selectable marker.

The resultant cells may be cells of the first, second, or third aspects of the invention, and so the method may be modified accordingly.

### Resistance to Horizontal Gene Transfer

The cells of the disclosure, including those of the first, second, and third aspect, may be resistant to horizontal gene transfer. For instance, the cells may be resistant to transfer of genetic information from mobile genetic elements, including plasmids (such as the F plasmid), viruses (including phages), and the like.

In particular, the cells of the present disclosure may be resistant to the transfer of genetic information from mobile genetic elements comprising relevant tRNAs. A relevant tRNA may be one that could decode one or more reassigned codon according to the canonical genetic code.

In some embodiments, the cells may reduce or may completely ablate transfer of a F plasmid comprising a relevant tRNA. This property can be testing using the methods of present FIG. 5C.

The cells may be bacteria and may be resistant to the bacteriophages disclosed in Nyerges et al. “Swapped genetic code blocks viral infections and gene transfer”. The cells may be E. coli and resistant to said bacteriophages.

The cells are also resistant to horizontal gene transfer from said cells into other types of cells. For instance, the cells of the first, second, or third aspect, or as created by methods of the fifth aspect, may be unable to transfer synthetic genes to wild-type bacteria or to bacteria. The cells of the present disclosure may be unable to transfer synthetic genes to wild-type bacteria of the same species. The synthetic genes may be according to any reassigned coding scheme as disclosed herein.

In addition, the cells of the present disclosure are also resistant to horizontal gene transfer from said cells to other cells not using the same reassigned coding scheme. Thus, the cells of the present disclosure are unable to transfer synthetic genes to bacteria not able to decode the synthetic gene according to the particular reassigned coding scheme. The other bacteria may also make use of a reassigned coding scheme but, if said scheme is orthogonal to the cells of the present disclosure, then horizontal gene transfer will be prevented.

Methods of Altering Susceptibility of a Gene to Mutations that Alter the Encoded Amino Acid Sequence

The cells, codes, and techniques disclosed herein enable methods for altering the susceptibility of a gene to mutations that alter the encoded amino acid sequence. Thus, the refactored codes disclosed herein may be used for accelerating or deaccelerating the rates of protein evolution.

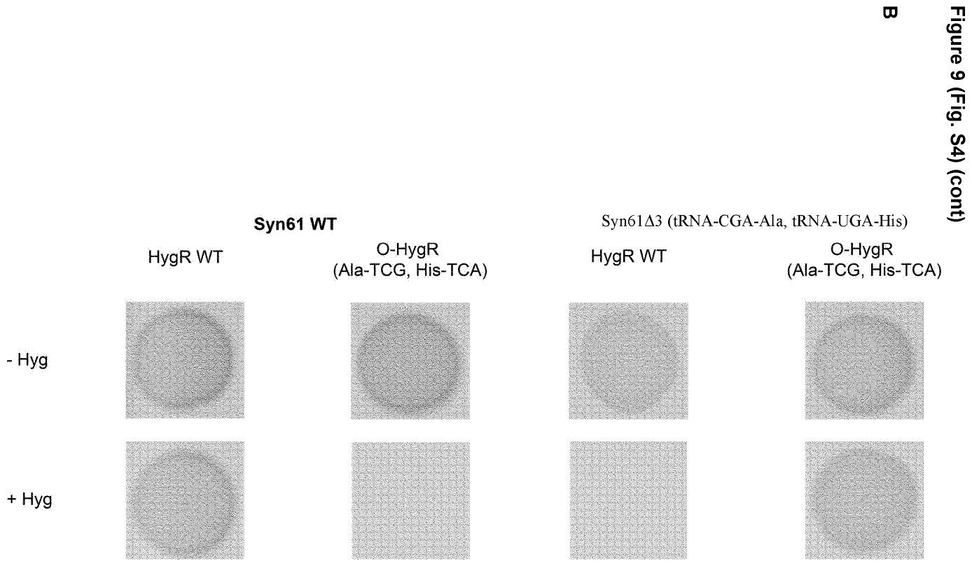

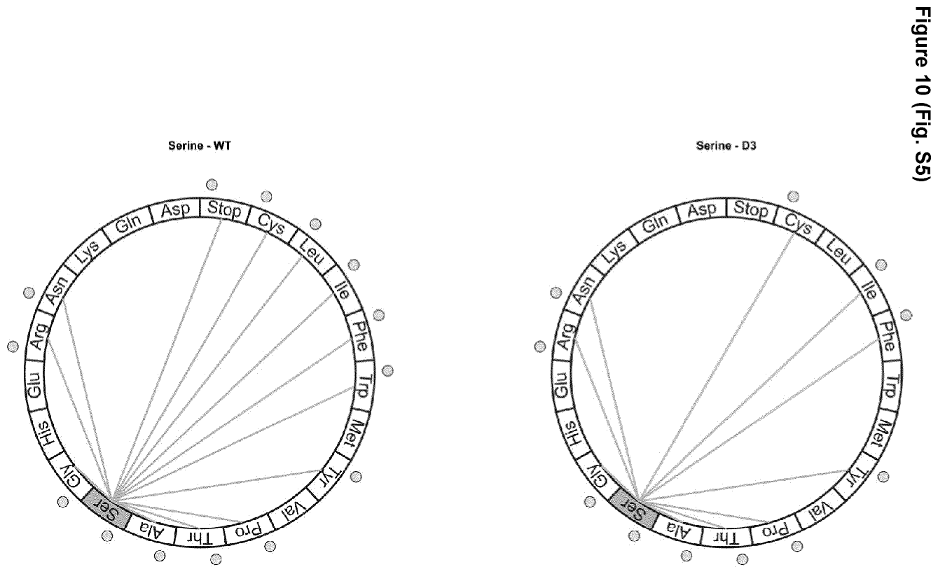

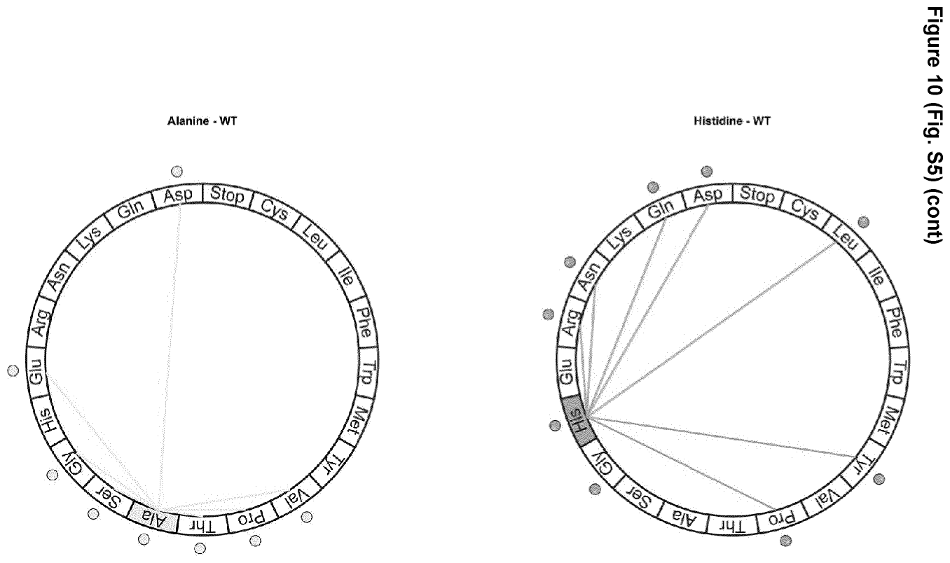

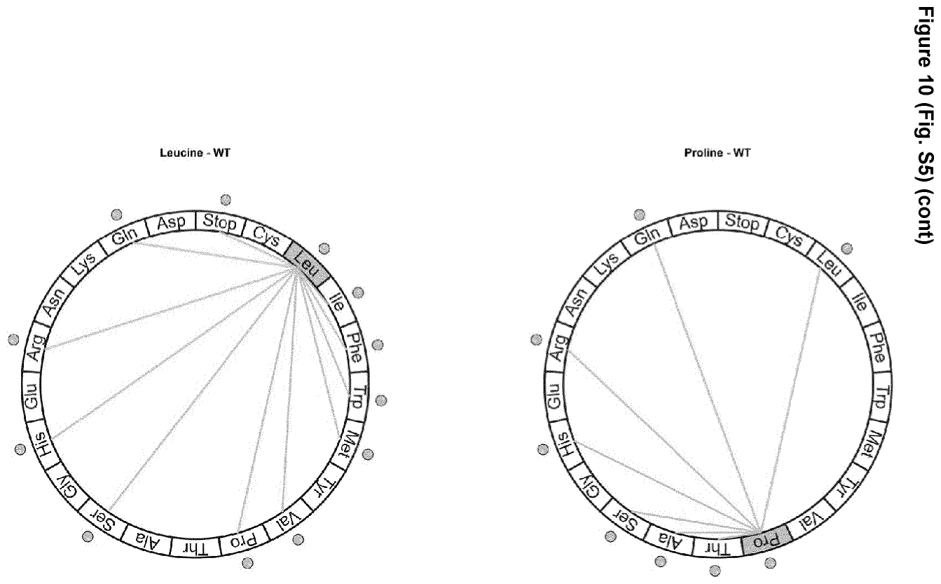

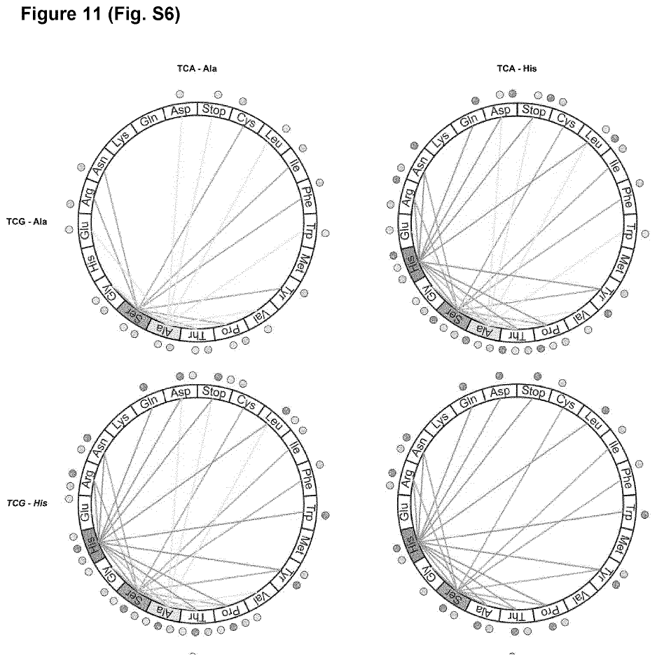

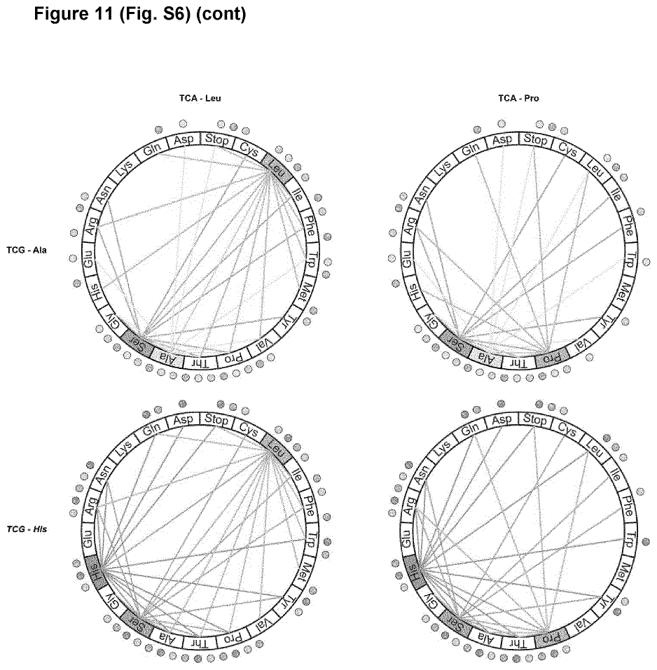

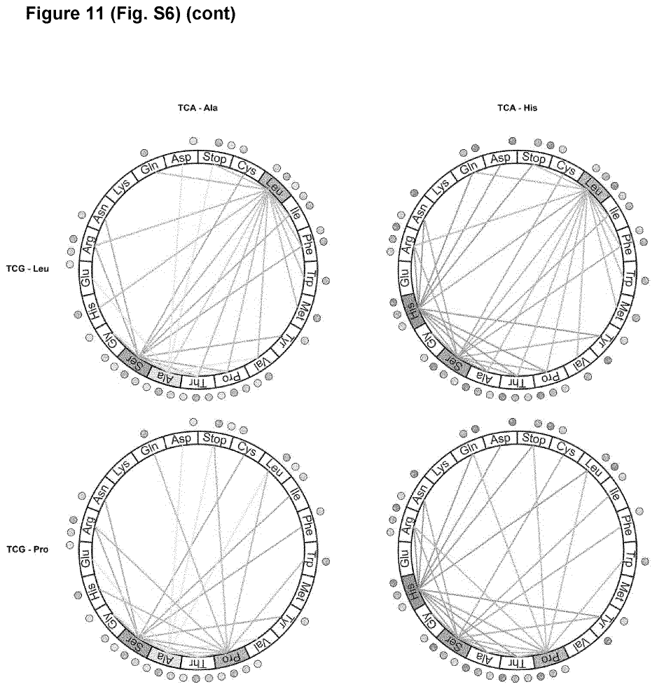

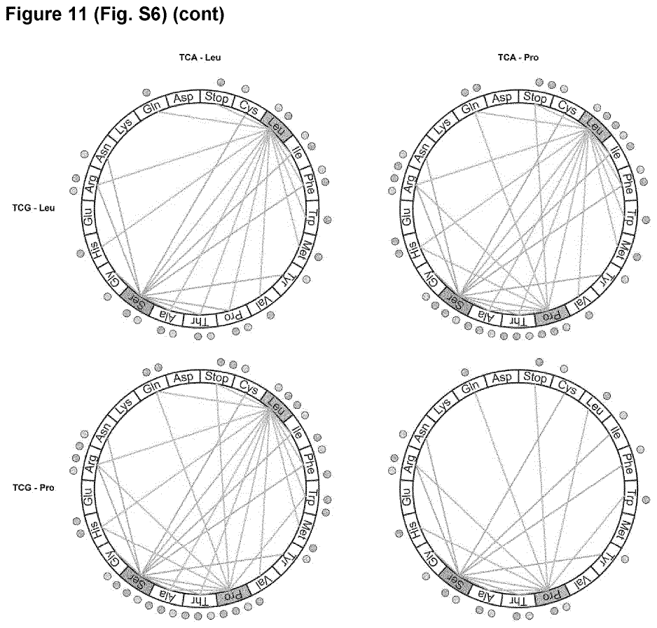

The canonical genetic code is, to a degree, conservative in that a point mutation may not alter the encoded amino acid. Additionally, a point mutation may alter the encoded amino acid to be another amino acid with similar properties (e.g. a conservative substitution) or dissimilar properties (e.g. a non-conservative substitution). The number of differences between types of codons may be varied and this may affect the chance that a point mutation will lead to: no change in encoded amino acid, a conservative change, or a non-conservative change. The inventors provide codes, and techniques for implementing such codes, that alter the mutational landscape (see, for instance, FIG. 10 (FIG. S5) and FIG. 11 (FIG. S6)).

Therefore, in one aspect, there is provided a method of altering susceptibility of a gene to mutations that alter the encoded amino acid sequence, the method comprising:
i) identifying a target gene; and
ii) incubating a cell comprising the target gene, wherein the cell comprises a tRNA capable of decoding at least one sense codon to a reassigned amino acid.

The target gene can be one or more target gene (or genes). The target gene can be a synthetic or natural gene. Suitably, a synthetic gene can alter the codon usage to favour evolutionary trajectories. In some aspects, the target gene may be according to a compressed genetic code.

The cell may be any as disclosed herein, for instance any of the first, second, or third aspect. The cell may be a bacterial cell such as E. coli, that has been recoded with respect to a first and a second type of sense codon. The cell may be Syn61, derived from Syn61, or recoded in the same manner as Syn61. The cell may be Syn61Δ3, derived from Syn61Δ3, or recoded in the same manner as Syn61Δ3.

In examples, the reassignment scheme may be any as illustrated in FIG. 2C. These schemes may be used to alter the mutational landscape as depicted in FIG. 11 (FIG. S6).

The cell may be incubated under conditions likely to or intended to cause mutations. The method may be for the purpose of evolving or improving a protein. The method may be for the purpose of rendering a target gene more resistant to mutation, for instance to protect the cell from harmful mutations.

### Recoding of Sense Codons

This section further describes exemplary embodiments of the recoding and is applicable to all aspects disclosed herein.

An endogenous tRNA is considered to be not expressed if the endogenous tRNA is not present in a form that would allow it to decode its cognate codon(s). Thus, an endogenous tRNA may be removed using any manner that would prevent the production of a functional form of the endogenous tRNA within the cell. For instance, the endogenous gene may be deleted or a portion of the gene may be deleted to prevent expression. Regulatory sequences may be deleted or altered to prevent expression. Alternatively, nonsense, frameshift, or missense mutations may prevent expression of the tRNA in a functional form.

“Recoding” as used herein, is the replacement of an occurrence of a type of codon with a different codon, such that the occurrence of the codon is removed from the genome. The recoded sense codon may be replaced with a synonymous codon to result in different codon usage without changing the encoded polypeptide. Alternatively, the sense codon may be replaced with a non-synonymous codon, for instance if the alteration in the sequence of the encoded polypeptide does not affect viability. The deleted endogenous tRNAs are those that are dispensable in light of the recoding. “Dispensable” as used herein, means not required for viability of the cell.

Viable cells are those that are capable of being metabolically active. In a particular embodiment, a viable cell may be capable of growth when cultured in an appropriate media and under appropriate conditions for the particular species or strain. Such cells may be referred to as capable of being cultured. As an example, if the cell is a bacterial cell such as E. coli, the assessment of viability may be performed by culturing said bacteria in a medium comprising LB medium, or on an agar comprising LB agar, at 37° C. The medium or agar may be supplemented with 2% glucose. Growth of the bacteria may be monitored using standard approaches, such as measurement of the OD₆₀₀. Alternative approaches, or approaches adapted to particular cells, bacteria strains, bacterial species, or in light of the inclusion of marker genes, are known to the skilled person.

A endogenous tRNA that decodes one or more sense codons that have been replaced (or deleted) may be deleted and the cell will remain viable if the tRNA decodes only the one or more sense codons that have been replaced (or deleted); or alternatively if the tRNA decodes one or more sense codons that have been replaced (or deleted) and one or more sense codons that have not been replaced (or deleted), if the tRNA is dispensable for the one or more sense codons that have not been replaced (or deleted) (i.e. the one or remaining sense codons which the tRNA decodes are decoded by one or more alternative tRNAs). For example, if the genome of a prokaryotic cell lacks TCA sense codons, serT, encoding tRNA_{Ser}^{UGA}, may be deleted and/or if the genome lacks TCG sense codons, serU, encoding tRNA_{Ser}^{CGA}, may be deleted. Thus, in an embodiment, the cell expresses neither tRNA_{Ser}^{UGA} nor tRNA_{Ser}^{CGA}.

The number of occurrences of the first and/or second type of sense codon that are recoded is adequate to enable the removal of the cognate tRNAs corresponding to said sense codons while maintaining viability of the cell. For example, this may be achieved by removing all of the natural occurrences of the first and second type of sense codon from the essential genes. In particular, a gene is considered essential if a “blank” codon (i.e. a codon for which the cell contains no corresponding tRNA or release factor) within the gene results in a loss of cell viability. Therefore, in an embodiment, all of the genes of the cell for which a blank codon could not be tolerated without a loss of viability are recoded, but genes that are able to tolerate blank codons may not be recoded. Thus, the skilled person can assess whether all of the essential genes have been recoded by assessing whether a cognate tRNA is dispensable. Notably, some embodiments require at least one essential gene to comprise the first and/or second type of sense codon; however, in such embodiments said codons are reassigned and not in a naturally occurring position.

The cells of the present disclosure, including the cells of the first, second, and third aspects, may be recoded with respect to a first, second, third, fourth, fifth, or further type of sense codon. The recoding of first and second types of sense codons is exemplified herein, and the skilled person would understand that the principle may be extended to recode, and hence reduce the occurrences of, further types of sense codon within the cell's genome. For example, further types of sense codon may be replaced by synonymous codons to remove particular occurrences without altering the encoded sequence, and adequate numbers of a particular type of sense codon may be removed such that at least one further endogenous tRNA is dispensable and need not be expressed by the cell.

In particular embodiments, the genome comprises 100 or more, 200 or more, or 300 or more essential genes with no natural occurrences of the first and/or second type of sense codon. For instance, all or substantially all of the essential genes in the genome may comprise no natural occurrences of the first and/or second type of sense codon.

In some embodiments, the essential genes may be selected from one or more of the list consisting of: ribF, ispA, ispH, dapB, folA, imp, yabQ, ftsL, ftsI, murE, murF, mraY, murD, ftsW, murG, murC, ftsQ, ftsA, ftsZ, lpxC, secM, secA, can, folK, hemL, yadR, dapD, map, rpsB, tsf, pyrH, frr, dxr, ispU, cdsA, yaeL, yaeT, lpxD, fabZ, lpxA, lpxB, dnaE, accA, tilS, proS, yafF, hemB, secD, secF, ribD, ribE, thiL, dxs, ispA, dnaX, adk, hemH, lpxH, cysS, folD, entD, mrdB, mrdA, nadD, holA, rlpB, leuS, lnt, ginS, fldA, cydA, infA, cydC, ftsK, lolA, serS, rpsA, msbA, lpxK, kdsB, mukF, mukE, mukB, asnS, fabA, mviN, rne, fabD, fabG, acpP, tmk, holB, lolC, loD, lolE, purB, minE, minD, pth, prsA, ispE, lolB, hemA, prfA, prmC, kdsA, topA, ribA, fabI, tyrS, ribC, ydiL, pheT, pheS, rplT, infC, thrS, nadE, gapA, yeaZ, aspS, argS, pgsA, yejM, metG, folE, yejM, gyrA, nrdA, nrdB, folC, accD, fabB, gltX, ligA, zipA, dapE, dapA, der, hisS, ispG, suhB, tadA, acpS, era, rnc, lepB, rpoE, pssA, yfiO, rplS, trmD, rpsP, ffh, grpE, csrA, ispF, ispD, ftsB, eno, pyrG, chpR, lgt, fbaA, pgk, yqgD, metK, yqgF, plsC, ygiT, parE, ribB, cca, ygjD, tdcF, yraL, yhbV, injB, nusA, ftsH, obgE, rpmA, rplU, ispB, murA, yrbB, yrbK, yhbN, rpsI, rplM, degS, mreD, mreC, mreB, accB, accC, yrdC, def, fmt, rplQ, rpoA, rpsD, rpsK, rpsM, secY, rplO, rpmD, rpsE, rplR, rplF, rpsH, rpsN, rplE, rplX, rplN, rpsQ, rpmC, rplP, rpsC, rplV, rpsS, rplB, rplW, rplD, rplC, rpsJ, fusA, rpsG, rpsL, trpS, yrfF, asd, rpoH, ftsX, ftsE, ftsY, yhhQ, bcsB, glyQ, gpsA, rfaK, kdtA, coaD, rpmB, dfp, dut, gmk, spoT, gyrB, dnaN, dnaA, rpmH, rnpA, yidC, tnaB, glmS, glmU, wzyE, hemD, hemC, yigP, ubiB, ubiD, hemG, yihA, ftsN, mur, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, and dnaC.

In particular, the essential genes may be selected from one or more of the list consisting of: ribF, ispA, ispH, dapB, folA, imp, yabQ, lpxC, secM, secA, can, folK, hemL, yadR, dapD, map, rpsB, tsf, pyrH, frr, dxr, ispU, cdsA, yaeL, yaeT, lpxD, fabZ, lpxA, lpxB, dnaE, accA, tilS, proS, yafF, hemB, secD, secF, ribD, ribE, thiL, dxs, ispA, dnaX, adk, hemH, lpxH, cysS, folD, entD, mrdB, mrdA, nadD, holA, rlpB, leuS, lnt, ginS, fldA, cydA, infA, cydC, ftsK, lolA, serS, rpsA, msbA, lpxK, kdsB, mukF, mukE, mukB, asnS, fabA, mviN, rne, fabD, fabG, acpP, tmk, holB, lolC, lolD, lolE, purB, minE, minD, pth, prsA, ispE, lolB, hemA, prfA, prmC, kdsA, topA, ribA, fabI, tyrS, ribC, ydiL, pheT, pheS, rplT, infC, thrS, nadE, gapA, yeaZ, aspS, argS, pgsA, yejM, metG, folE, yejM, gyrA, nrdA, nrdB, folC, accD, fabB, gltX, ligA, zipA, dapE, dapA, der, hisS, ispG, suhB, tadA, acpS, era, rnc, lepB, rpoE, pssA, yfiO, rplS, trmD, rpsP, ffh, grpE, csrA, ispF, ispD, ftsB, eno, pyrG, chpR, lgt, fbaA, pgk, yqgD, metK, yqgF, plsC, ygiT, parE, ribB, cca, ygjD, tdcF, yraL, yhbV, injB, nusA, ftsH, obgE, rpmA, rplU, ispB, murA, yrbB, yrbK, yhbN, rpsI, rplM, degS, mreD, mreC, mreB, accB, accC, yrdC, def, fmt, rplQ, rpoA, rpsD, rpsK, rpsM, secY, rplO, rpmD, rpsE, rplR, rplF, rpsH, rpsN, rplE, rplX, rplN, rpsQ, rpmC, rplP, rpsC, rplV, rpsS, rplB, rplW, rplD, rplC, rpsJ, fusA, rpsG, rpsL, trpS, yrfF, asd, rpoH, ftsX, ftsE, ftsY, yhhQ, bcsB, glyQ, gpsA, rfaK, kdtA, coaD, rpmB, dfp, dut, gmk, spoT, gyrB, dnaN, dnaA, rpmH, rnpA, yidC, tnaB, glmS, glmU, wzyE, hemD, hemC, yigP, ubiB, ubiD, hemG, yihA, ftsN, mur, murB, birA, secE, nusG, rplJ, rplL, rpoB, rpoC, ubiA, plsB, lexA, dnaB, ssb, alsK, groS, psd, orn, yjeE, rpsR, chpS, ppa, valS, yjgP, yjgQ, and dnaC.

In other embodiments, the cell may comprise a genome comprising 5 or fewer natural occurrences of the first and/or second type of sense codon. The genome may be derived from a parent genome and may comprise less than 10%, 5%, 2%, 1%, 0.5%, 0.1% of the occurrences of the first and/or second type of sense codon, relative to the parent genome. The genome may comprise 100 or more, 200 or more, or 1000 or more genes with no natural occurrences of the first and/or second type of sense codon. In particular, all or substantially all the genes in the genome may have no natural occurrences of the first and/or second type of sense codon. Thus, the genome of the cell may comprise 5, 4, 3, 2, 1, or no natural occurrences of a first type of sense codon and 5, 4, 3, 2, 1, or no natural occurrences of a second type of sense codon.

The genome may be derived from a parent genome and comprise 5 or fewer (e.g. 5, 4, 3, 2, 1), or no natural occurrences of native sense codons of the first and/or second type. In a particular embodiment, the genome is derived from a parent genome and comprises no natural occurrences of native sense codons of the first and the second type. In some embodiments the genome comprises 100 or more, 200 or more, 300 or more, 400 or more, 500 or more, 600 or more, 700 or more, 800 or more, 900 or more, 1000 or more, 1500 or more, or 2000 or more recoded genes. In some embodiments the genes are those for which there is evidence of translation and/or of the predicted protein product. For example, the genome may comprise 100 or more, 200 or more, 300 or more, 400 or more, 500 or more 600 or more, 700 or more, 800 or more, 900 or more, 1000 or more, 1500 or more, or 2000 or more recoded genes for which there is evidence of translation and/or of the predicted protein product.

In an embodiment, all annotated open reading frames within the genome have no natural occurrences of the sense codons of the first and the second type. The cell may be a bacterial cell, preferably E. coli, and the genome of the E. coli may contain no natural occurrences of a first and a second type of sense codon as annotated in GenBank accession number CP040347.1.

In a particular embodiment, the protein-encoding genes have no natural occurrences of the sense codons of the first and the second type. In particular embodiments, no proteins are translated from any of the remaining natural occurrences of the first and/or second type of sense codon and/or genes comprising the remaining natural occurrences of the first and/or second type of sense codons are putative or are non-coding genes. In some embodiments the translation of the genes comprising the remaining natural occurrences of the first and/or second type of sense codons is reduced and/or prevented (e.g. the genes may comprise stop codons in the 5′ sequence).

Any remaining natural occurrences of the sense codons may be necessary to ensure that the genome is viable. For example, one or more, in particular all, of the remaining natural occurrences of the first and/or second type of sense codons in the genome may be present in the regulatory elements of essential genes; and/or one or more, in particular all, of the remaining natural occurrences of the first and/or second type of sense codons may be in genes in which there is no evidence for translation or the predicted protein product (i.e. putative or non-coding genes).

As used herein, a “sense codon” is a nucleotide triplet that codes for an amino acid. Thus, sense codons may be identified in a genome by gene prediction, i.e. by identifying regions of the genome that code for proteins (i.e. genes) and the corresponding open reading frames (ORFs). Typically, genomes naturally comprise 61 sense codons: GCT, GCC, GCA, GCG, CGT, CGC, CGA, CGG, AGA, AGG, AAT, AAC, GAT, GAC, TGT, TGC, CAA, CAG, GAA, GAG, GGT, GGC, GGA, GGG, CAT, CAC, ATT, ATC, ATA, TTA, TTG, CTT, CTC, CTA, CTG, AAA, AAG, ATG, TTT, TTC, CCT, CCC, CCA, CCG, TCT, TCC, TCA, TCG, AGT, AGC, ACT, ACC, ACA, ACG, TGG, TAT, TAC, GTT, GTC, GTA, and GTG (read from 5′ to 3′ on the coding strand of DNA). The standard genetic code encodes the 20 canonical amino acids using the 61 triplet codons. 18 of the 20 amino acids are encoded by more than one synonymous codon. The first or second type of sense codon may be native sense codons, i.e. sense codons which are present in the parent genome.

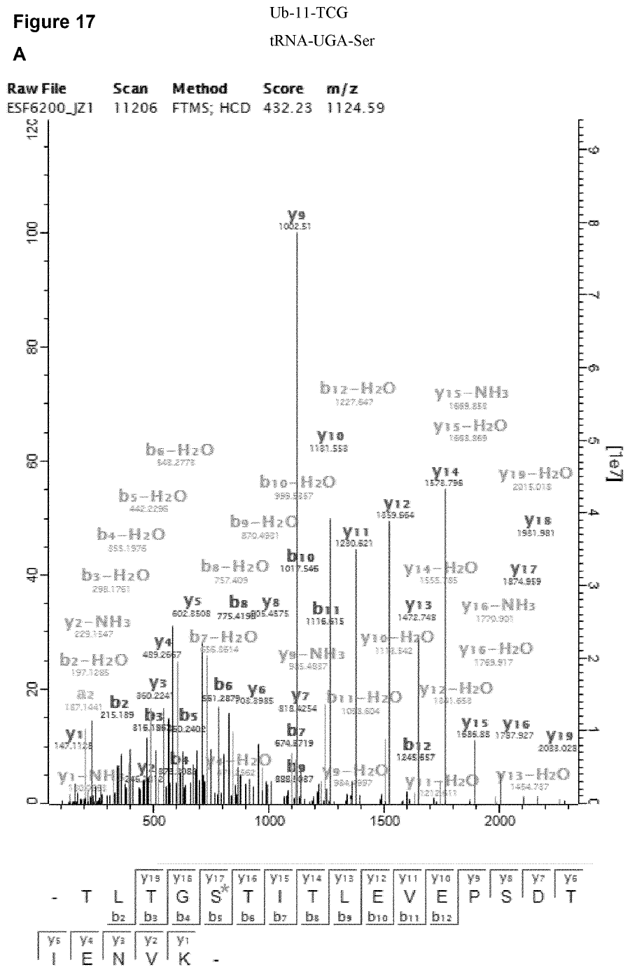

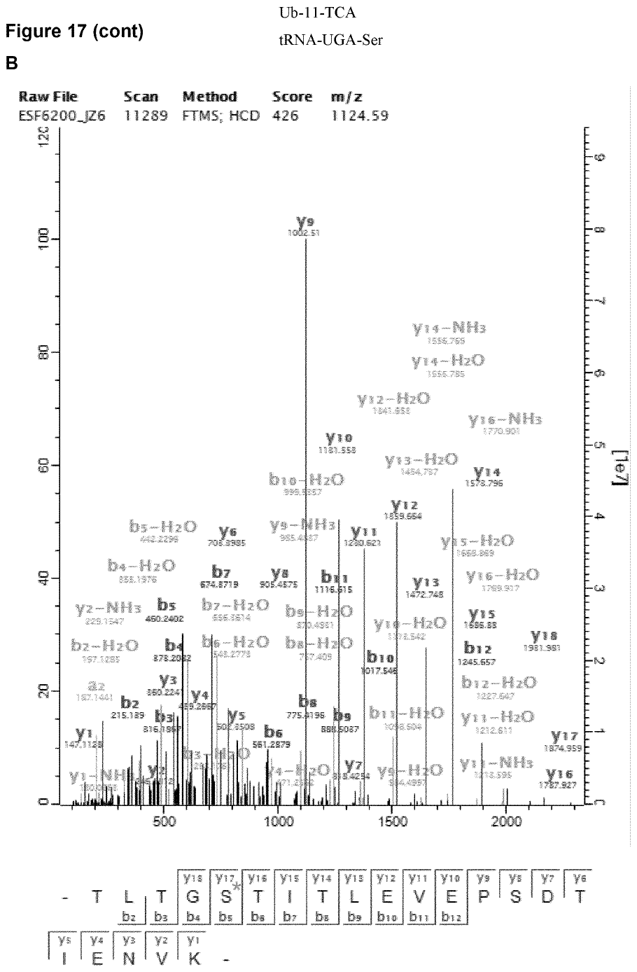

The 61 sense codons in DNA are transcribed into corresponding mRNA and subsequently decoded by one or more tRNAs. tRNAs carry an amino acid to a ribosome as directed by the sense codons in the mRNA. The tRNAs can recognise one or more sense codons via a complementary anticodon. A sequence of sense codons is subsequently translated into a polypeptide (i.e. a sequence of amino acids). Codon and anticodon interactions in the E. coli genome are shown in FIG. 17 of WO2020/229592 (incorporated herein by reference).

The genome wide removal of the first and/or second type of sense codon, but not other sense codons, enables cognate tRNAs corresponding to said first or second type of sense codons to be deleted without removing the ability to decode the sense codons remaining in the genome.

The recoded sense codons may be selected from: TCG, TCA, TCT, TCC, AGT, or AGC. In a particular embodiment, the first and second type of sense codon are TCA and TCG.

To achieve removal of sense codons they may be replaced with synonymous sense codons. This is preferable to ensure that the encoded protein sequence is not changed. For instance, the cell may have a genome wherein 90% or more, 95% or more, 98% or more, 99% or more, 99.5% or more, 99.6% or more, 99.7% or more, 99.8% or more, 99.9% or more, or 100% of the occurrences of the first or second type of sense codons in the parent genome is replaced with synonymous sense codons. The person skilled in the art is able to deduce suitable synonymous sense codon replacements. For example, in E. coli, typically TCG, TCA, TCT, TCC, AGT and AGC all encode serine.

In some embodiments, the replacement is a defined replacement, i.e., one sense codon is replaced with a single synonymous sense codon. Preferably, 90% or more, 95% or more, 98% or more, 99% or more, 99.5% or more, 99.6% or more, 99.7% or more, 99.8% or more, 99.9% or more, or 100% of the natural occurrences of the first or second type of sense codon in the parent genome are is replaced with a defined (i.e. single) synonymous sense codon.

For example, the defined replacement may be: TCG replaced with any one of TCT, TCC, AGT, or AGC; or TCA replaced with any one of TCT, TCC, AGT, or AGC. In particular, the replacements are selected from one or more of: TCG to either AGT or AGC; or TCA to either AGT or AGC. In a particular embodiment, TCG is replaced with AGC and TCA is replaced with AGT.

Preferably, none of these codon replacements affect ribosomal binding sites (AGGAGG), which are highly conserved regulatory sequences in E. coli. The selected codon replacements may be tested on a small test region (e.g. a 20 kb region of the genome rich in both essential target genes and target codons) to assess viability. If the codon replacements are not viable on the small test region they may be disregarded.

When replacement of sense codons in the parent genome with defined replacement synonymous sense codons does not result in a viable cell, alternative replacement synonymous sense codons may be used. For instance, 99.9% of the occurrences of the first and/or second type of sense codon in the parent genome may be replaced with a defined (i.e. single) synonymous sense codon, and the remaining 0.1% with alternative synonymous sense codons. For example, 99.9% of the natural occurrences of TCG may be replaced with AGC and 0.1% replaced with TCT, TCC, AGT or AGC; and/or 99.9% of the occurrences of TCA may be replaced with AGT and 0.1% replaced with TCT, TCC, AGT or AGC.

In some instances, a particular occurrence of a sense codon may not be replaceable with any of the potential synonymous sense codons without affecting viability. To retain viability, the sense codon may be replaced with a non-synonymous sense codon that does not affect viability. For instance, 99.9% of the occurrences of the first and/or second type of sense codon in the parent genome may be replaced with a defined (i.e. single) synonymous sense codon, and the remaining 0.1% with alternative non-synonymous sense codons.

### Recoding of Stop Codons

This section further describes exemplary embodiments involving recoding stop codons, and is applicable to all aspects disclosed herein.

In some examples, a first type of stop codon has been recoded within the genome of the cell such that the first endogenous release factor is dispensable, and the cell does not express a first endogenous release factor.

The removal of the first endogenous release factor may performed in cells wherein the genomes have been recoded to remove occurrences of a first type of stop codon. Optionally the removed stop codons are replaced with synonymous codons. The deleted endogenous release factor is the factor that is dispensable in light of the recoding.

In a particular examples, the cell does not express a first endogenous tRNA, a second endogenous tRNA, and a first endogenous release factor; and the genome has been recoded to remove a plurality of the sense codons for which the first and second endogenous tRNAs are cognate, and to remove a plurality of the stop codon for which the first endogenous release factor is cognate.

An endogenous release factor is considered to be not expressed if the endogenous release factor is not present in a form that would allow it to decode its cognate codon(s). Thus, an endogenous release factor may be removed using any manner that would prevent the production of a functional form of the endogenous release factor within the cell. For instance, the endogenous gene may be deleted or a portion of the gene may be deleted to prevent expression. Regulatory sequences may be deleted or altered to prevent expression. Alternatively, nonsense, frameshift, or missense mutations may prevent expression of the release factor in a functional form.

As used herein, a “stop codon” is a nucleotide triplet that codes for termination of translation into proteins. Typically, genomes naturally comprise 3 stop codons: TAA (“ochre”), TGA (“opal” or “umber”) and TAG (“amber”).

The number of natural occurrences of the first type of stop codon that are removed is adequate to enable the removal of the cognate release factor corresponding to said stop codons while maintaining viability of the cell. Thus, in some examples, the essential genes of the cell do not contain occurrences of the first type of stop codon. The essential genes may be any as discussed herein, particularly those discussed in relation to the removal of the first or second type of sense codon. In particular examples, the genome comprises 100 or more, 200 or more, or 300 or more essential genes with no natural occurrences of the first type of stop codon. For instance, all or substantially all of the essential genes in the genome may comprise no occurrences of the first type of stop codon.

For example, the genome may comprise 100 or more, 200 or more, or 300 or more essential genes with no natural occurrences of the first type of sense codon, the second type of sense codon, and the first type of stop codon. In particular, all or substantially all of the essential genes in the genome may comprise no natural occurrences of the first type of sense codon, the second type of sense codon, and the first type of stop codon.

In some embodiments, the genome comprises 10 or fewer, 5 or fewer, or no natural occurrences of the first type of stop codon. Such as 5, 4, 3, 2, 1, or no natural instances of the first type of stop codon.

In a particular example the first type of stop codon is TAG and the first endogenous release factor is RF-1. In such examples, there may be 10 or fewer, 5 or fewer, or no natural occurrences of the amber stop codon (TAG). In other examples, 90% or more, 95% or more, 98% or more, 99% or more, or all of the occurrences of TAG in the parent genome are replaced with TAA (the ochre stop codon). In particular embodiments, the genome comprises no occurrences of the amber stop codon (TAG), optionally wherein all of the occurrences of TAG in the parent genome are replaced with TAA (the ochre stop codon).

In an embodiment, all annotated open reading frames within the genome have no occurrences of the first type of stop codon. The cell of the present disclosure may be a bacterial cell, such as E. coli, and the genome of the E. coli may contain no occurrences of first type of stop codon as annotated in GenBank accession number CP040347.1.

In some examples, the protein-encoding genes have no natural occurrences of the first type of stop codon. In particular examples, no proteins are translated from any of the remaining occurrences of the first type of stop codon and/or genes comprising the remaining occurrences of the first type of stop codon are putative or are non-coding genes. In some examples the translation of the genes comprising the remaining occurrences of the first type of stop codon is reduced and/or prevented (e.g. the genes may comprise stop codons in the 5′ sequence).

Any remaining occurrences of the first type of stop codon may be necessary to ensure that the genome is viable. For example, one or more, in particular all, of the remaining natural occurrences of the first type of stop codon in the genome may be present in the regulatory elements of essential genes; and/or one or more, in particular all, of the remaining occurrences of the first type of stop codon may be in genes in which there is no evidence for translation or the predicted protein product (i.e. putative or non-coding genes).

### Genomes Recoded for Sense Codons and Stop Codons

This section further describes exemplary embodiments of the recoding, and is applicable to the all aspects disclosed herein.

Accordingly, in some examples the genome comprises no occurrences of a first and a second type of sense codon, and no occurrences of one stop codon, preferably the amber stop codon (TAG). In particular examples, the genome comprises no occurrences of the sense codons TCG and TCA, and no occurrences of the amber stop codon (TAG), optionally wherein TCG, TCA and TAG in the parent genome are replaced with synonymous codons, for example 99.9% or more of the occurrences of TCG in the parent genome are replaced with AGC, 99.9% or more of the occurrences of TCA in the parent genome are replaced with AGT and all of the occurrences of TAG in the parent genome are replaced with TAA.

In a particular example, the genome of the cell has been recoded such that the sense codon TCG has been replaced with AGC, the sense codon TCA has been replaced with AGT, and the stop codon TAG has been replaced with TAA, and wherein sufficient numbers of said codons have been recoded such that two cognate tRNAs and a cognate release factor are dispensable.

In a particular example, the cell of the present disclosure is an E. coli cell that does not express tRNA^{Ser}_{UGA}, tRNA^{Ser}_{CGA}, or RF-1, occurrences of the sense codon TCA have been recoded such that tRNA^{Ser}_{UGA} is dispensable (e.g. occurrences of TCA in essential genes of the parent strain have been replaced with AGT), occurrences of the sense codon TCG have been recoded such that tRNA^{Ser}_{CGA} is dispensable (e.g. occurrences of TCG in essential genes of the parent strain have been replaced with AGC), and occurrences of the stop codon TAG have been recoded such that RF-1 is dispensable (e.g. occurrences of TAG in essential genes of the parent strain have been replaced with TAA).

In some embodiments the genome of the cell of the present disclosure comprises a polynucleotide sequence which is at least 80%, 85%, 90%, 95%, 98%, 99%, 99.5%, 99.8%, 99.9%, or 100% identical to the sequence provided in GenBank accession number CP040347.1, and wherein the genome has been further altered such that tRNA^{Ser}_{UGA} and tRNA^{Ser}_{CGA} are not functionally expressed (for instance, by deleting serT and serU). The genome may have been even further altered such that RF-1 is not functionally expressed (for instance, by deleting prfA). An E. coli strain comprising a genome according to GenBank accession number CP040347.1 is referred to as Syn61 WT in the Examples disclosed herein.

In some embodiments the genome of the cell of the present disclosure comprises a polynucleotide sequence which is at least 80%, 85%, 90%, 95%, 98%, 99%, 99.5%, 99.8%, 99.9%, or 100% identical to SEQ ID NO: 1, and wherein the genome has been further altered such that tRNA^{Ser}_{UGA} and tRNA^{Ser}_{CGA} are not functionally expressed (for instance, by deleting serT and serU). The genome may have been even further altered such that RF-1 is not functionally expressed (for instance, by deleting prfA). An E. coli strain comprising a genome according to SEQ ID NO: 1 may be referred to as Syn61(ev1).

In some embodiments the genome of the cell of the present disclosure comprises a polynucleotide sequence which is at least 80%, 85%, 90%, 95%, 98%, 99%, 99.5%, 99.8%, 99.9%, or 100% identical to SEQ ID NO: 2, and wherein the genome has been further altered such that tRNA^{Ser}_{UGA} and tRNA^{Ser}_{CGA} are not functionally expressed (for instance, by deleting serT and serU). The genome may have been even further altered such that RF-1 is not functionally expressed (for instance, by deleting prfA). An E. coli strain comprising a genome according to SEQ ID NO: 2 may be referred to as Syn61(ev2).

In some embodiments the genome of the cell comprises a polynucleotide sequence which is at least 80%, 85%, 90%, 95%, 98%, 99%, 99.5%, 99.8%, 99.9%, or 100% identical to SEQ ID NO: 3. An E. coli strain comprising a genome according to SEQ ID NO: 3 may be referred to as Syn61Δ3.

In some embodiments the genome of the cell comprises a polynucleotide sequence which is at least 80%, 85%, 90%, 95%, 98%, 99%, 99.5%, 99.8%, 99.9%, or 100% identical to SEQ ID NO: 4. An E. coli strain comprising a genome according to SEQ ID NO: 4 may be referred to as Syn61Δ3(ev3).

In some embodiments the genome of the cell comprises a polynucleotide sequence which is at least 80%, 85%, 90%, 95%, 98%, 99%, 99.5%, 99.8%, 99.9%, or 100% identical to SEQ ID NO: 5. An E. coli strain comprising a genome according to SEQ ID NO: 5 may be referred to as Syn61Δ3(ev4).

In some embodiments the genome of the cell comprises a polynucleotide sequence which is at least 80%, 85%, 90%, 95%, 98%, 99%, 99.5%, 99.8%, 99.9%, or 100% identical to SEQ ID NO: 6 (also provided as GenBank accession number CP071799.1). An E. coli strain comprising a genome according to SEQ ID NO: 6 may be referred to as Syn61Δ3(ev5).

There is provided herein a prokaryotic cell comprising a genome which is at least 80%, 85%, 90%, 95%, 98%, 99%, 99.5%, 99.8%, 99.9%, or 100% identical to any one of: SEQ ID NO: 1, SEQ ID NO: 2, SEQ ID NO: 3, SEQ ID NO: 4, SEQ ID NO: 5, and SEQ ID NO: 6. The prokaryotic cell may be a bacterium, for instance E. coli. In some embodiments, the calculation of the sequence identity percentage excludes any sequence that has been inserted to further modify the cells. The calculation of sequence identity percentage may further exclude any exogenous sequences that have been further introduced into the genome. For instance, any additional tRNAs, selection markers, changes to genes required for viability according to the present disclosure, constructs for the industrial expression of peptide or protein products, etc.

### Species of Cells

The cell of the present disclosure, including those of all aspects disclosed herein, may be a prokaryotic cell. The bacterial cell may be of any species suitable for heterologous protein production, in particular the production of polypeptides. Suitable bacterial host cells include: Escherichia (e.g. Escherichia coli), caulobacteria (e.g. Caulobacter crescentus), phototrophic bacteria (e.g. Rhodobacter sphaeroides), cold adapted bacteria (e.g. Pseudoalteromonas haloplanktis, Shewanella sp. strain Ac10), pseudomonads (e.g. Pseudomonas fluorescens, Pseudomonas putida, Pseudomonas aeruginosa), halophilic bacteria (e.g. Halomonas elongate, Chromohalobacter salexigens), streptomycetes (e.g. Streptomyces lividans, Streptomyces griseus), Nocardia (e.g. Nocardia lactamdurans), mycobacteria (e.g. Mycobacterium smegmatis), coryneform bacteria (e.g. Corynebacterium glutamicum, Corynebacterium ammoniagenes, Brevibacterium lactofermentum), bacilli (e.g. Bacillus subtilis, Bacillus brevis, Bacillus megaterium, Bacillus licheniformis, Bacillus amyloliquefaciens), vibrio bacteria (e.g. Vibrio cholera, Vibrio natriegens), and lactic acid bacteria (e.g. Lactococcus lactis, Lactobacillus plantarum, Lactobacillus casei, Lactobacillus reuteri, Lactobacillus gasseri). In some examples, the bacterium is a gram-negative bacterium.

In particular examples, the bacterium is an Escherichia coli, Salmonella enterica, or Shigella dysenteriae. More preferably, the cell is an E. coli. Suitable E. coli cells include K-12, MG1655, BL21, BL21(DE3), AD494, Origami, HMS174, BLR(DE3), HMS174(DE3), Tuner(DE3), Origami2(DE3), Rosetta2(DE3), Lemo21(DE3), NiCo21(DE3), T7 Express, SHuffle Express, C41(DE3), C43(DE3), and m15 pREP4 or derivatives thereof (Rosano, G. L. and Ceccarelli, E. A., 2014. Frontiers in microbiology, 5, p. 172). In particular, the cell may be MG1655 or BL21, or a derivative thereof. MG1655 is considered as the wild type strain of E. coli. The GenBank ID of genomic sequence of this strain is U00096. BL21 is widely available commercially. For example, it can be purchased from New England BioLabs with catalog number C2530H.

The cell may contain a genome which is derived from the same species or strain, or may be derived from a different species. For example, if the cell is E. coli the genome may be an E. coli genome.

The cell of the present disclosure, including those of all aspects disclosed herein, may be biocontained cells. Thus, the cells of the present disclosure may only be viable or capable of proliferation under conditions that are not found in nature. Such cells may be considered to comprise a biocontainment system.

For instance, the cells may be viable or capable of proliferating only in the presence of an agent that is not found in natural environments. Such cells are capable of being cultured in the presence of said agent but, if the cells were to be placed into an environment lacking the agent, would not be maintained as a population of cells. Examples of such agents includes unnatural amino acids, which may be required for functional translation of one or more essential gene. Other examples include ligands required for the expression or activity of essential genes/proteins.

In another example, the cells may comprise a gene that prevents viability or the ability to proliferate, wherein the gene is inactive in the presence of an agent that is not found in natural environments. This gene may be referred to as a “kill switch” and may, for example, encode a toxin.

### Production of Polymers

As disclosed herein, the cells of the present disclosure may be suitable for polymer production. Thus, in a seventh aspect of the present disclosure, there is provided use of any cell disclosed herein for the production of a polymer.

In an embodiment, there is provided a method for making a polymer, the method comprising: culturing a cell as disclosed herein, providing the cell with a nucleic acid sequence encoding the polymer, and obtaining the polymer.

The polymer may be a polypeptide. The polymer may be a heterologous protein. The polymer may comprise monomers that can be incorporated by a charged-tRNA, such as canonical amino acids, natural amino acids, unnatural amino acids, beta amino acids, hydroxy acids, alpha hydroxy acids, and the like.

### Further Information

Sequence comparisons can be conducted with the aid of readily available sequence comparison programs. These publicly and commercially available computer programs can calculate sequence identity between two or more sequences.

The skilled technician will appreciate how to calculate the percentage identity between two nucleic sequences. In order to calculate the percentage identity between two nucleic sequences, an alignment of the two sequences must first be prepared, followed by calculation of the sequence identity value. The percentage identity for two sequences may take different values depending on: (i) the method used to align the sequences, for example, the Needleman-Wunsch algorithm (e.g. as applied by Needle(EMBOSS) or Stretcher(EMBOSS), the Smith-Waterman algorithm (e.g. as applied by Water(EMBOSS)), or the LALIGN application (e.g. as applied by Matcher(EMBOSS); and (ii) the parameters used by the alignment method, for example, local versus global alignment, the matrix used, and the parameters applied to gaps.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (iv) the number of equivalenced positions excluding overhangs. Furthermore, it will be appreciated that percentage identity is also strongly length-dependent. Therefore, the shorter a pair of sequences is, the higher the sequence identity one may expect to occur by chance.

A calculation of percentage identities between two nucleic acid sequences may then be calculated from such an alignment as (N/T)*100, where N is the number of positions at which the sequences share an identical residue, and T is the total number of positions compared including gaps but excluding overhangs.

The sequence alignment may be a pairwise sequence alignment. Suitable services include Needle (EMBOSS), Stretcher (EMBOSS), Water (EMBOSS), Matcher (EMBOSS), LALIGN, or GeneWise. In an example, the identity between two amino acid sequences may be calculated using the service Needle(EMBOSS) set to the default parameters, e.g. matrix (BLOSUM62), gap open (10), gap extend (0.5), end gap penalty (false), end gap open (10), and end gap extend (0.5). In another example, the identity between two amino acid sequences may be calculated using the service Matcher (EMBOSS) set to the default parameters, e.g. matrix (BLOSUM62), gap open (14), gap extend (4), alternative matches (1). In an example, the identity between two nucleic acid sequences may be calculated using the service Needle(EMBOSS) set to the default parameters, e.g. matrix (DNAfull), gap open (10), gap extend (0.5), end gap penalty (false), end gap open (10), and end gap extend (0.5). In another example, the identity between two nucleic acid sequences may be calculated using the service Matcher (EMBOSS) set to the default parameters, e.g. matrix (DNAfull), gap open (16), gap extend (4), alternative matches (1).

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made to the Examples, which are not intended to limit the invention in any way.

Some embodiments of the invention may be defined by the following clauses.

1. A cell that:
comprises a genome wherein at least a first type of sense codon has been recoded such that a first endogenous tRNA is dispensable;
does not express the first endogenous tRNA;
expresses a first modified tRNA capable of decoding the first type of sense codon, wherein the first modified tRNA is charged with a first amino acid that is not a naturally cognate amino acid for the first type of sense codon; and
comprises a gene required for viability, wherein the gene comprises at least one occurrence of the first type of sense codon and the cell is viable when the first type of sense codon in said gene is decoded as the first amino acid.

2. The cell of clause 1, wherein the cell is not viable if the first type of sense codon in the gene required for viability is decoded according to the canonical genetic code, or wherein the first type of sense codon in the gene required for viability at least partially contributes to a loss of viability if decoded according to the canonical genetic code.

3. The cell of clause 1 or clause 2, wherein the gene required for viability is an essential gene or a positive selectable marker.

4. The cell of any one of clauses 1 to 3, wherein the first amino acid is a naturally occurring amino acid.

5. The cell of any one of clauses 1 to 4, wherein a second type of sense codon has been recoded within the genome; optionally wherein a second endogenous tRNA is dispensable and the cell does not express the second endogenous tRNA; and optionally wherein the cell expresses a second modified tRNA, which is capable of decoding the second type of sense codon, wherein the second modified tRNA is charged with a second amino acid that is not a naturally cognate amino acid for the second type of sense codon.

6. The cell of clause 5, wherein a gene required for viability comprises at least one occurrence of the second type of sense codon and the cell is viable when the second type of sense codon in said gene is decoded as the second amino acid.

7. The cell of clause 6, wherein the cell is not viable if the second type of sense codon in the gene required for viability is decoded according to the canonical genetic code, or wherein the second type of sense codon at least partially contributes to a loss of viability if decoded according to the canonical genetic code.

8. The cell of any one of clauses 5 to 7, wherein the second amino acid is a naturally occurring amino acid.

9. The cell of any one of clauses 1 to 8, wherein the cell is viable when its genes are decoded by the modified tRNA(s) and is non-viable when its genes are decoded at least partially according to the canonical genetic code.

10. A cell with increased resistance to horizontal gene transfer or mobile genetic elements, wherein the cell has been modified to reassign at least one type of sense codon to an amino acid not associated with the sense codon in the canonical genetic code, and the cell comprises a gene required for viability that is functional when decoded according to the reassigned genetic code and is not functional when decoded according to the canonical genetic code.

11. A cell that:
comprises a genome wherein a first type of sense codon and a second type of sense codon have been recoded such that a first endogenous tRNA and a second endogenous tRNA are dispensable;
does not express the first endogenous tRNA and the second endogenous tRNA;
expresses a first anticodon-swapped tRNA derived from a naturally occurring first parent tRNA, wherein the first anticodon-swapped tRNA is charged with a first amino acid and the first parent tRNA is an isoacceptor for the first amino acid, and wherein the first amino acid is not a naturally cognate amino acid for the first type of sense codon; and
expresses a second anticodon-swapped tRNA derived from a naturally occurring second parent tRNA, wherein the second anticodon-swapped tRNA is charged with a second amino acid and the second parent tRNA is an isoacceptor for the second amino acid, and wherein the second amino acid is not a naturally cognate amino acid for the second type of sense codon;
   wherein the first and/or second modified tRNA cannot decode any type of codon apart from the first type of sense codon and/or the second type of sense codon.

12. The cell of clause 11, wherein:
the first and second type of sense codon would canonically be decoded by the same tRNA or overlapping tRNAs due to wobble base pairing, wherein the first anticodon-swapped tRNA cannot decode any codon type apart from the first type of sense codon and/or the second anticodon-swapped tRNA cannot decode any codon type apart from the second type of sense codon; and/or
the first type of sense codon and the second type of sense codon are of the formula XXN, and wherein the first anticodon-swapped tRNA cannot decode the second type of sense codon, and the second anticodon-swapped tRNA cannot decode the first type of sense codon.

13. The cell of clause 11 or clause 12, wherein the first amino acid and the second amino acid are different types of amino acid.

14. The cell of any one of clauses 11 to 13, wherein the first and second parent tRNAs are derived from the same cell type as the cell of clause 11; optionally wherein the first and/or second anticodon-swapped tRNA comprise identity elements that are recognised by an aminoacyl-tRNA synthetase endogenous to the cell.

15. The cell of any one of clauses 11 to 14, wherein the first and the second type of sense codon canonically encode serine, the first and the second type of sense codon canonically encode alanine, or the first and the second type of sense codon canonically encode leucine.

16. The cell of any one of clauses 11 to 15, wherein the first and/or second anticodon-swapped tRNA does not decode TCC or TCT codons.

17. The cell of any one of clauses 11 to 16, wherein the first and/or second amino acid is a naturally occurring amino acid; optionally wherein the first amino acid is any one of alanine, histidine, leucine, and proline; and/or the second amino acid is any one of alanine, histidine, leucine, and proline.

18. A cell that:
comprises a genome wherein a first type of sense codon and a second type of sense codon have been recoded such that a first endogenous tRNA and a second endogenous tRNA are dispensable;
does not express the first endogenous tRNA and the second endogenous tRNA;
expresses a first modified tRNA capable of decoding the first type of sense codon, wherein the first modified tRNA is charged with a first amino acid that is not a naturally cognate amino acid for the first type of sense codon; and
expresses a second modified tRNA capable of decoding the second type of sense codon, wherein the second modified tRNA is charged with a second amino acid that is not a naturally cognate amino acid for the second type of sense codon;
   wherein:
i) the first amino acid is alanine and the second amino acid is alanine;
ii) the first amino acid is alanine and the second amino acid is histidine;
iii) the first amino acid is alanine and the second amino acid is leucine;
iv) the first amino acid is alanine and the second amino acid is proline;
v) the first amino acid is histidine and the second amino acid is alanine;
vi) the first amino acid is histidine and the second amino acid is histidine;
vii) the first amino acid is histidine and the second amino acid is leucine;
viii) the first amino acid is histidine and the second amino acid is proline;
ix) the first amino acid is leucine and the second amino acid is alanine;
x) the first amino acid is leucine and the second amino acid is histidine;
xi) the first amino acid is leucine and the second amino acid is proline;
xii) the first amino acid is proline and the second amino acid is alanine;
xiii) the first amino acid is proline and the second amino acid is histidine;
xiv) the first amino acid is proline and the second amino acid is leucine; or
xv) the first amino acid is proline and the second amino acid is proline.

19. The cell of clause 18, wherein
the first modified tRNA cannot decode the second type of sense codon and/or the second modified tRNA cannot decode the first type of sense codon; and/or
the first modified tRNA cannot decode any type of codon apart from the first type of sense codon and/or the second modified tRNA cannot decode any type of codon apart from the second type of sense codon.

20. The cell of clause 18 or clause 19, wherein the first modified tRNA is an anticodon-swapped tRNA canonically associated with the first amino acid, and/or the second modified tRNA is an anticodon-swapped tRNA canonically associated with the second amino acid.

21. The cell of any one of clauses 18 to 20, wherein
the first modified tRNA is derived from a tRNA that is endogenous to the cell and is an isoacceptor for the first amino acid, or is derived from a tRNA found in a mobile genetic element and is an isoacceptor for the first amino acid; and/or
the second modified tRNA is derived from a tRNA that is endogenous to the cell and is an isoacceptor for the second amino acid, or is derived from a tRNA found in a mobile genetic element and is an isoacceptor for the second amino acid; and/or
the first and/or second modified tRNA comprise identity elements that are recognised by an aminoacyl-tRNA synthetase endogenous to the cell.

22. The cell of any one of clauses 18 to 21, wherein the first and the second type of sense codon canonically encode serine.

23. The cell of any one of clauses 18 to 22, wherein the first type of sense codon is TCA and/or the second type of sense codon is TCG.

24. The cell of any preceding clause, wherein the cell is prokaryotic cell, a bacterial cell, or an Escherichia coli cell.

25. A method of increasing the resistance of a cell to mobile genetic elements or horizontal gene transfer, wherein the cell has been modified to reassign at least one type of sense codon to an amino acid not associated with the sense codon in the canonical genetic code, said method comprising:
modifying a gene required for viability to include at least one occurrence of the reassigned sense codon, wherein
the cell is viable if the reassigned sense codon in said gene is decoded as the reassigned amino acid, and
the cell is not viable if the reassigned sense codon in said gene is decoded according to the canonical genetic code, or wherein the reassigned sense codon in said gene at least partially contributes to a loss of viability if decoded according to the canonical genetic code.

### EXAMPLES

### Summary

The near-universal genetic code defines the correspondence between codons in genes and amino acids in proteins. It is widely hypothesized that refactoring the structure of the genetic code will create organisms with new properties, and may create a genetic firewall to limit the escape of genetic information from synthetic organisms. However, it has been impossible to test these hypotheses. Here we create refactored genetic code/decoder systems, which—unlike code-compressed organisms—exhibit semantic- and functional-orthogonality with respect to the code/decoder system for the canonical code. We thereby create orthogonal, and mutually orthogonal, horizontal gene transfer systems, which permit the transfer of genetic information between organisms that use the same genetic code, but restrict transfer of genetic information between cells that use different genetic codes. Moreover, we show that locking an orthogonal code into synthetic organisms completely blocks invasion by mobile genetic elements that successfully invade code-compressed organisms.

To elaborate, we show that code compressed genes are read in natural cells, such that code compression cannot limit the escape of genes from engineered organisms into the biosphere. Moreover, we show that mobile genetic elements that use the canonical genetic code, and carry the tRNA decoders necessary to complement the tRNAs absent in the recipient cell, can invade Syn61Δ3 cells. We reassign sense codons to alternative canonical amino acids in Syn61Δ3, and thereby refactor the structure of the genetic code; we create 16 refactored codes with features not found in nature. We demonstrate that code reassignment enables the creation of synthetic genes, written in new codes, which are correctly read in synthetic organisms with cognate decoders, but incorrectly read in natural cells. We also show that genes written in the canonical code, which are correctly read in natural organisms, are incorrectly read in the synthetic organism. The genetic code-decoder system in the synthetic organism exhibits semantic- and functional-orthogonality with respect to the code-decoder system for the canonical code. We leverage this orthogonality to create orthogonal, and mutually orthogonal, horizontal gene transfer systems that permit the horizontal transfer of genetic information between cells that use the same genetic code, but restrict horizontal transfer of genetic information between cells that use different genetic codes. Moreover, we show that locking an orthogonal code into the synthetic organism completely blocks invasion by mobile genetic elements that successfully invade code-compressed organisms.

### Example 1—Compressed Codes are Non-Orthogonal

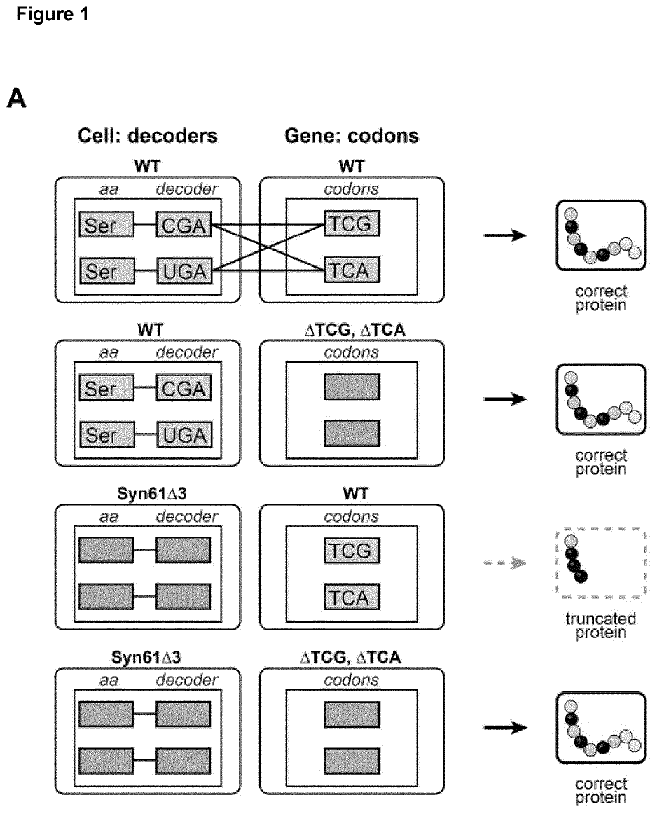

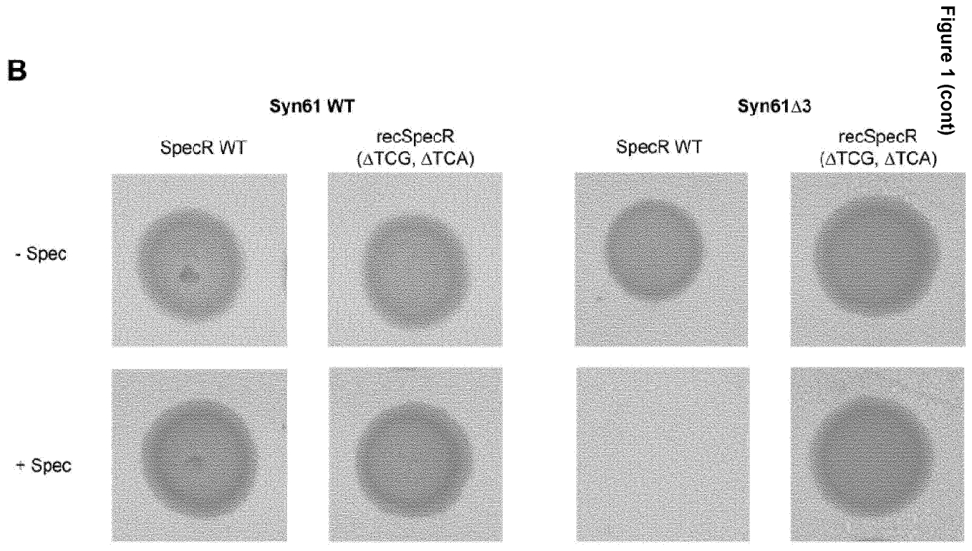

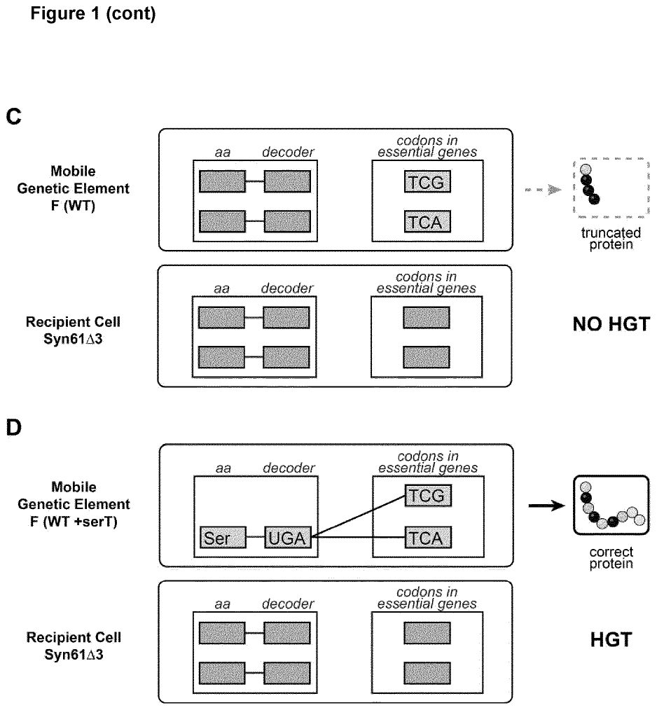

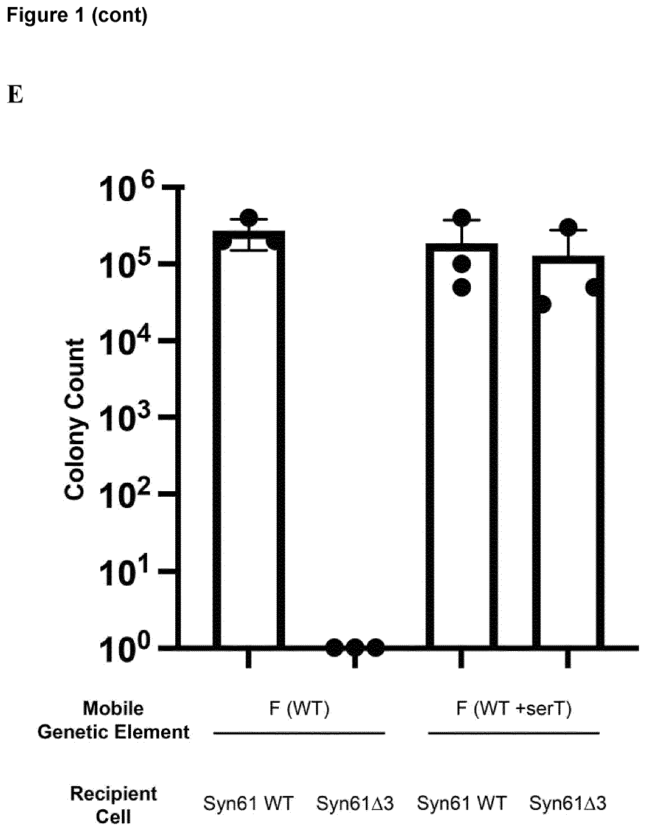

A spectinomycin resistance gene written in the canonical genetic code (SpecR WT) was correctly read in cells that contain the full complement of tRNAs to read the canonical code, and conferred spectinomycin resistance to WT cells (Syn61 WT). However, consistent with previous observations (18), SpecR WT did not confer spectinomycin resistance to Syn61Δ3 cells, (FIG. 1), as Syn61Δ3 does not read all the codons in the canonical genetic code.

We created a spectinomycin resistance gene (recSpecR (ΔTCG, TCA)), written using the compressed genetic code we used to create Syn61 (TCG and TCA codons were replaced with AGC and AGT respectively, and the TAG stop codon was replaced with TAA). recSpecR (ΔTCG, TCA) conferred spectinomycin resistance to Syn61Δ3 cells, which use the same codon compression scheme as recSpecR (ΔTCG, TCA) in their genome (FIG. 1). The recSpecR (ΔTCG, TCA) gene also conferred spectinomycin resistance to cells that read the canonical genetic code; this was expected, as the compressed genetic code uses a subset of the codons used in the canonical genetic code. We made similar observations with codon compressed- and wt-hygromycin resistance genes (FIG. S1).

These experiments demonstrated that genetic information written in the canonical code can be read in WT cells, but not in cells with genome-wide code compression and cognate tRNA deletion. However, code compressed genes can be read in both cells with genomic code compression and cognate tRNA deletion and in WT cells. The codons used in the compressed genetic code are not orthogonal with respect to the tRNA decoders in WT cells. Therefore, there is no barrier limiting genetic information from engineered biological cells—that use compressed genetic codes—being read by natural forms of life that use the canonical code. Creating orthogonal genetic codes, with active barriers restricting the transfer of genetic information from engineered biological systems to natural systems, is an important and unaddressed challenge.

### Example 2—tRNAs Enable Invasion of Codon Compressed Organisms

A WT F plasmid (F (WT), which uses the canonical genetic code, was efficiently transferred to WT cells. In contrast, F (WT) was not transferred to Syn61Δ3 (FIG. 1), as expected. However, upon selecting for the conjugation of F (WT) from WT cells into Syn61Δ2 cells (Syn61 cells deleted for serU and serT but containing prfA), we obtained two viable colonies in which recipient cells had received F (WT) (FIG. S2). These colonies corresponded to rare events, appearing at a frequency 10⁶ fold lower than the colonies resulting from conjugation of F (WT) into WT cells. Sequencing the two clones revealed that they had acquired sequences containing serT from the donor cell. This provided direct experimental evidence that selection for transfer of a mobile genetic element that uses the canonical code into cells can enable the selection for the tRNA genes required to read the canonical genetic code.

To follow the effects of introducing serT into recipient cells in a reproducible system, we created the mobile genetic element F (WT+serT), a variant of F (WT) that contains serT. We demonstrated that F (WT+serT) can be transferred to Syn61Δ3 cells, and that this transfer is dependent on serT (FIG. 1). We conclude that acquisition of serT is sufficient to circumvent genetic isolation provided by codon compression and cognate tRNA deletion in Syn61Δ3. These experiments highlight that creating systems that actively obstruct invasion by mobile genetic elements that carry their own decoders is an important challenge.

### Example 3—Refactoring Code-Structure

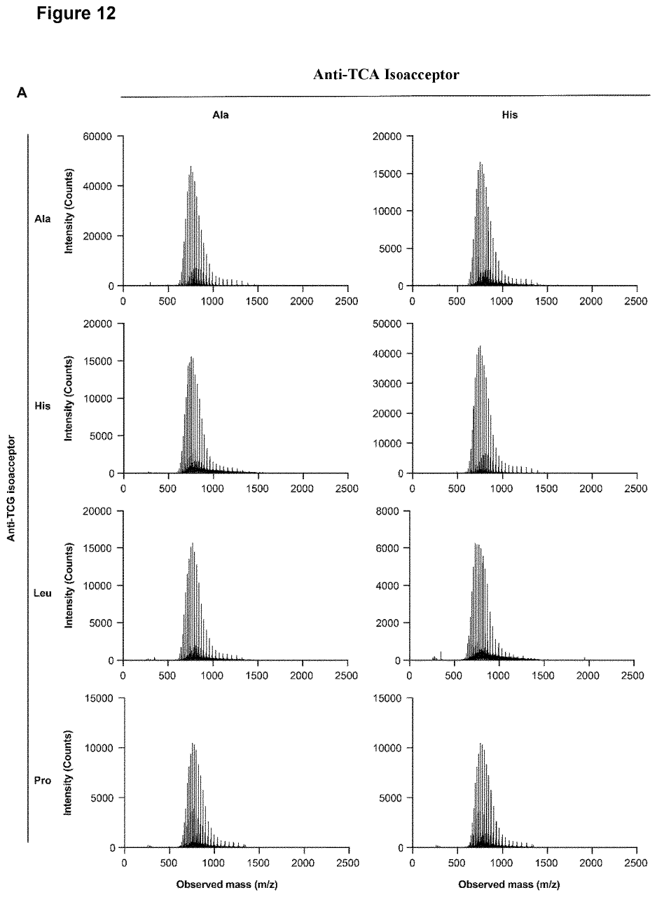

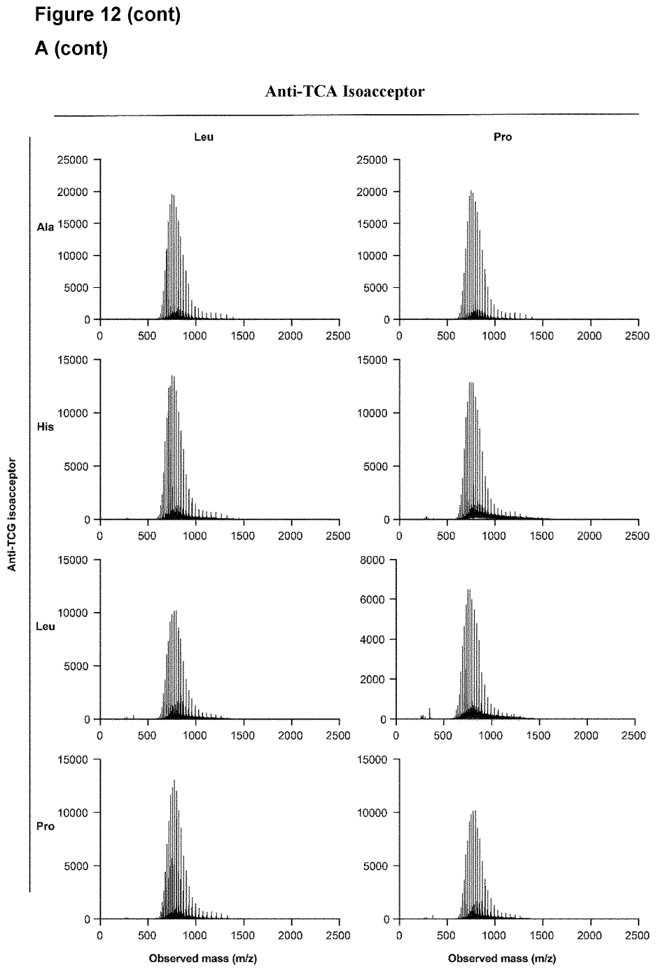

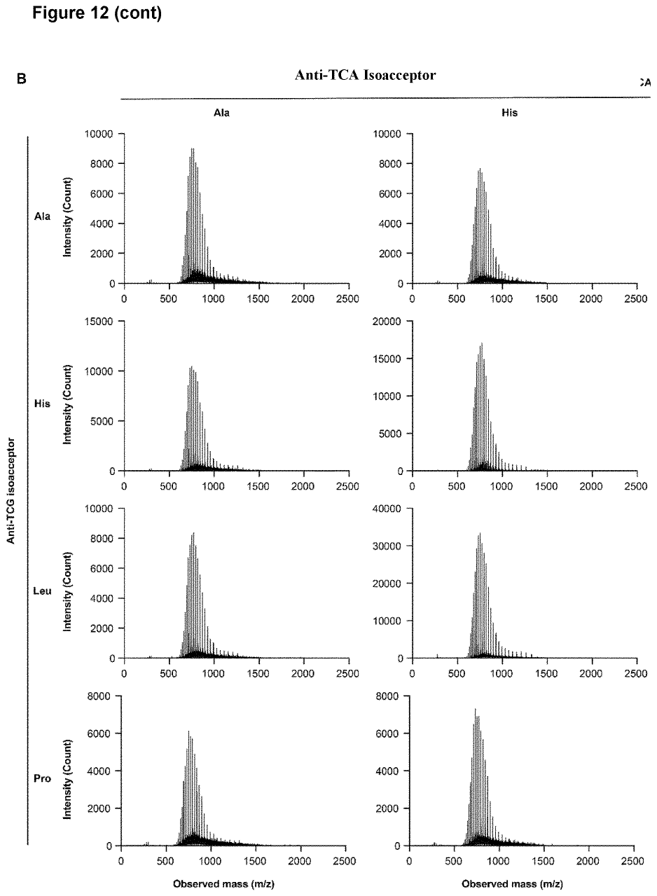

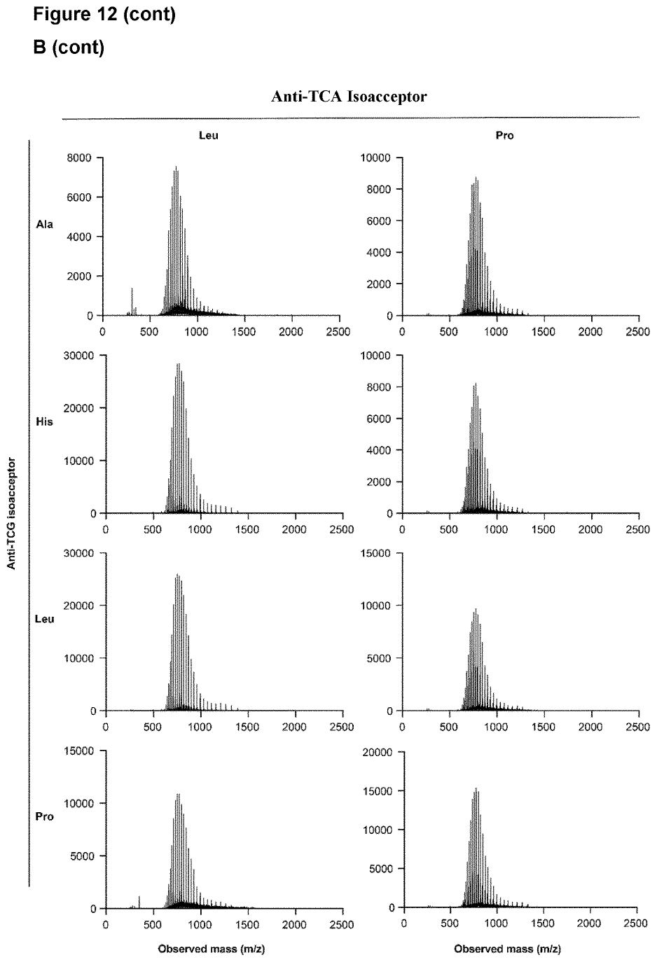

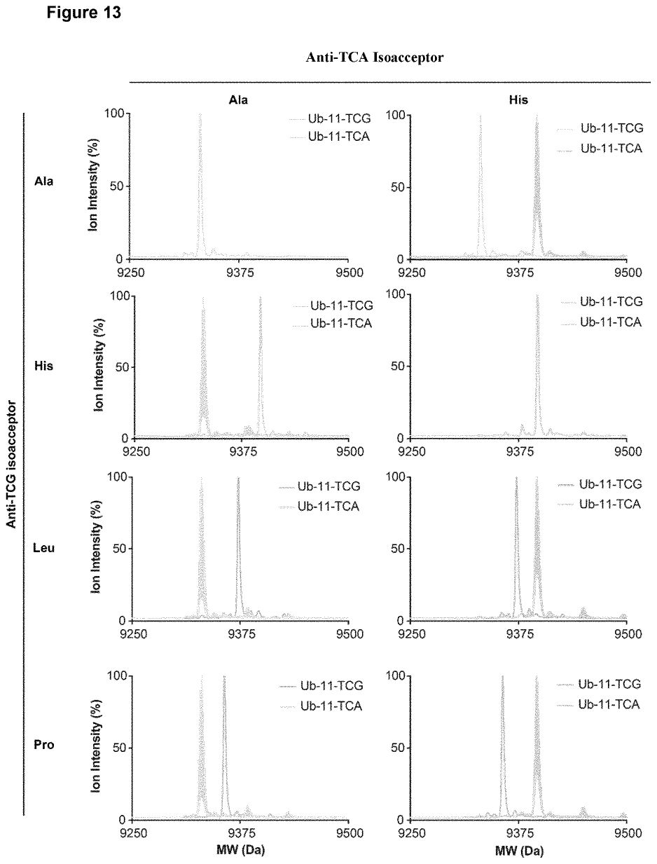

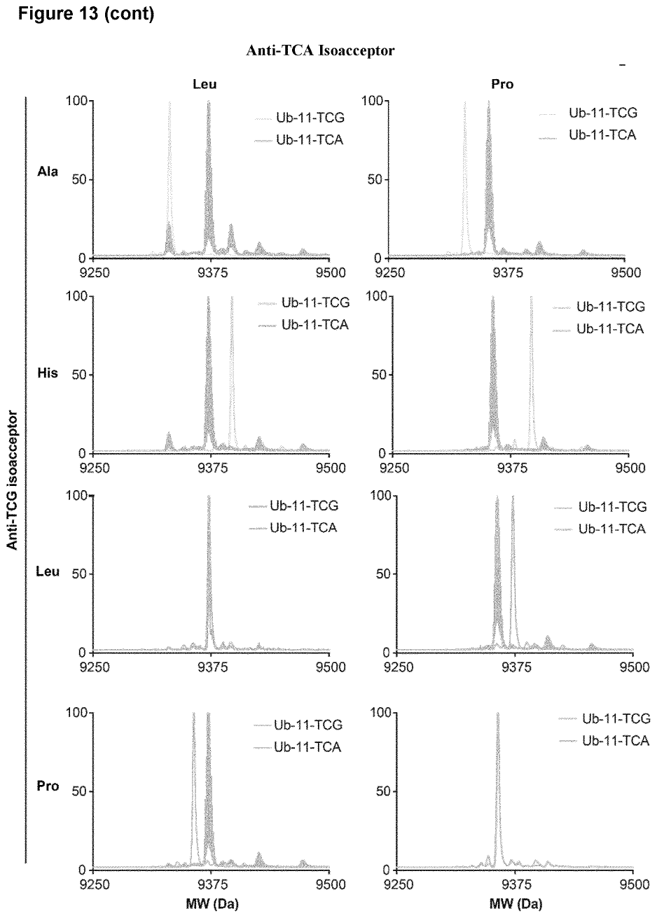

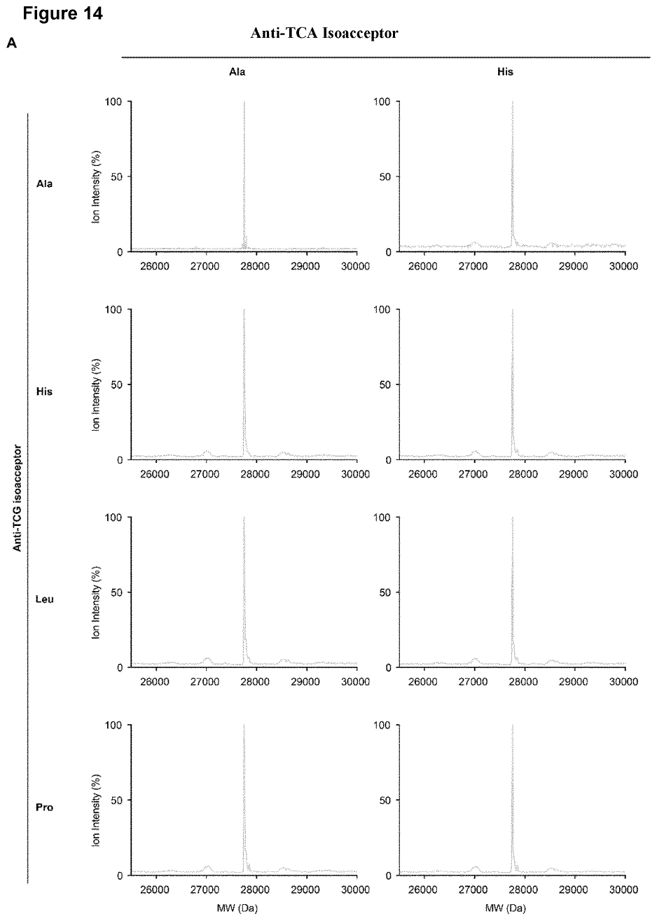

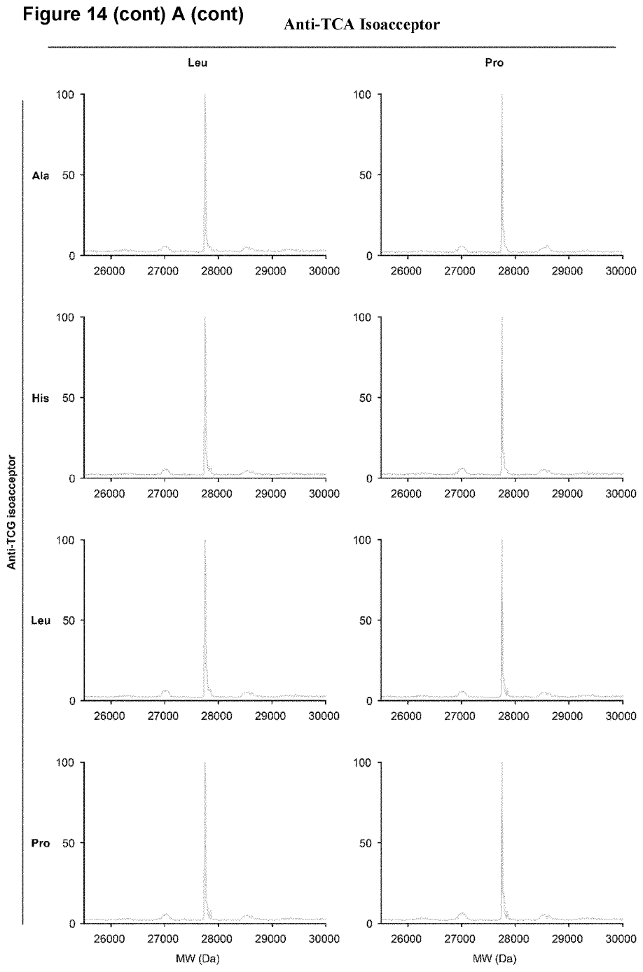

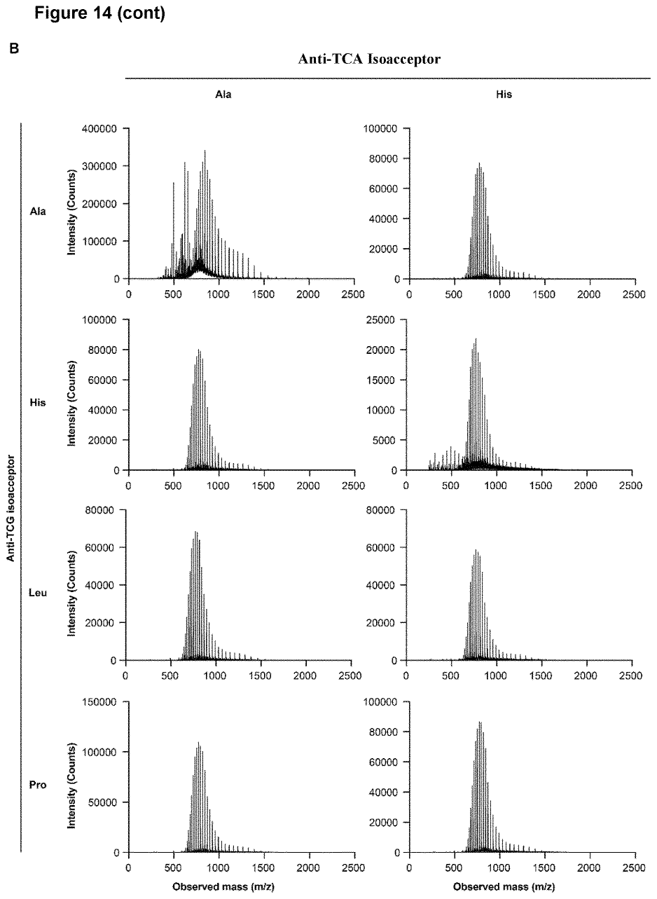

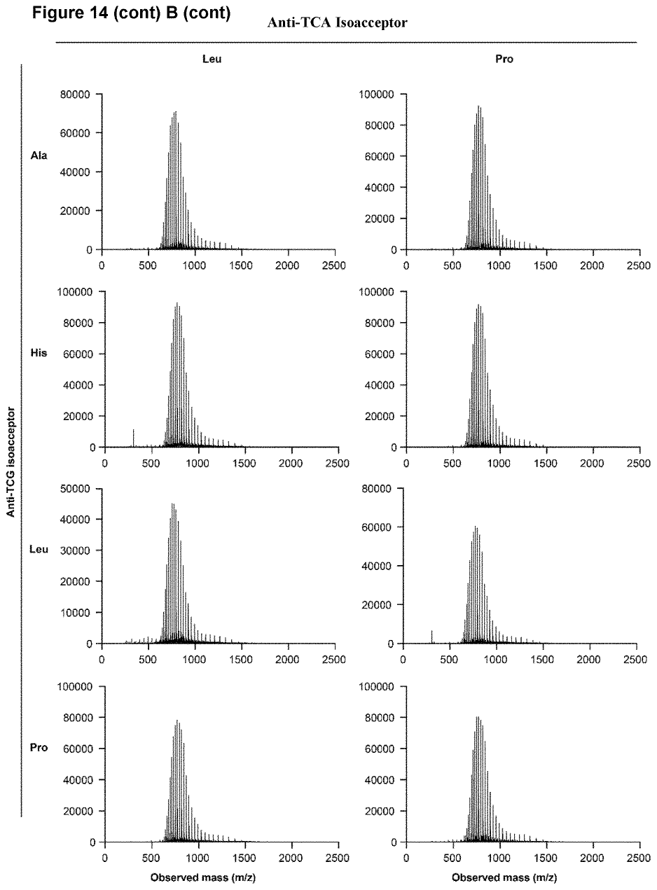

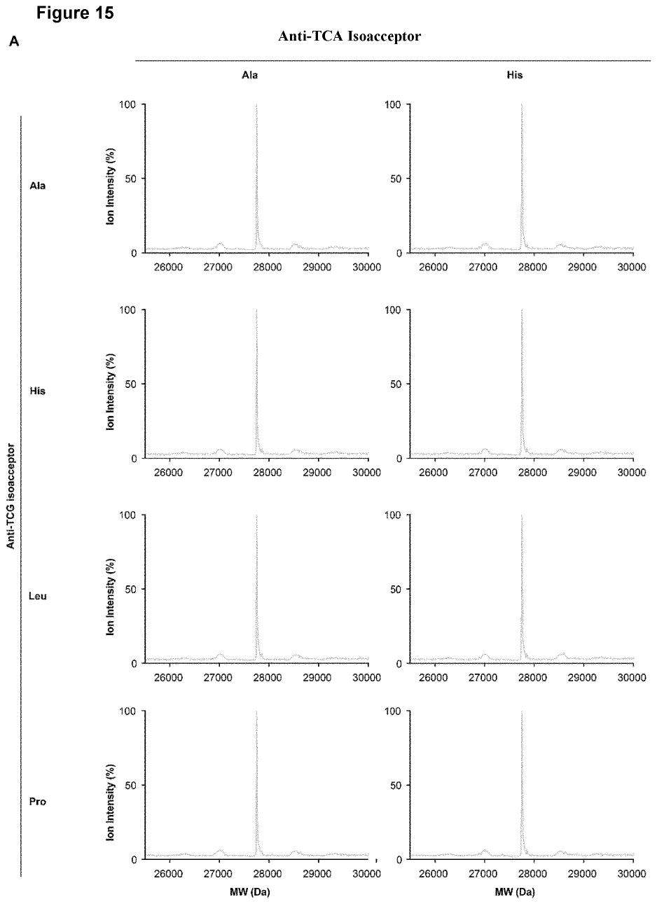

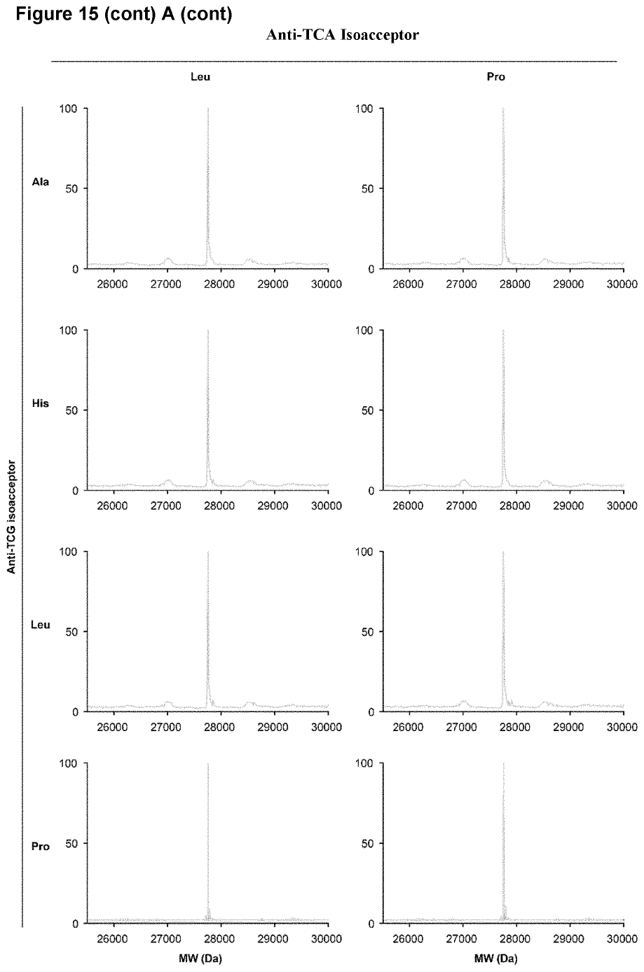

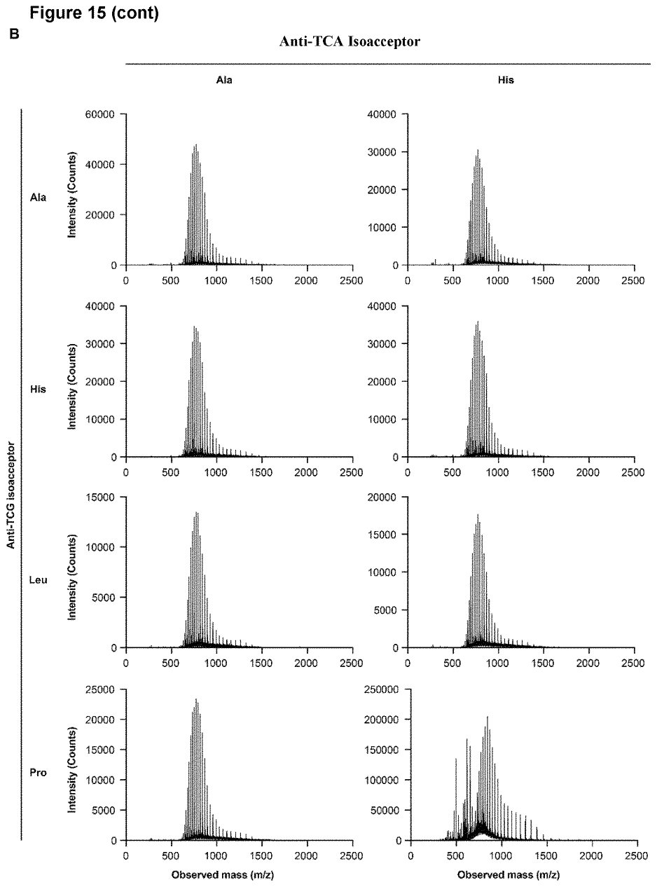

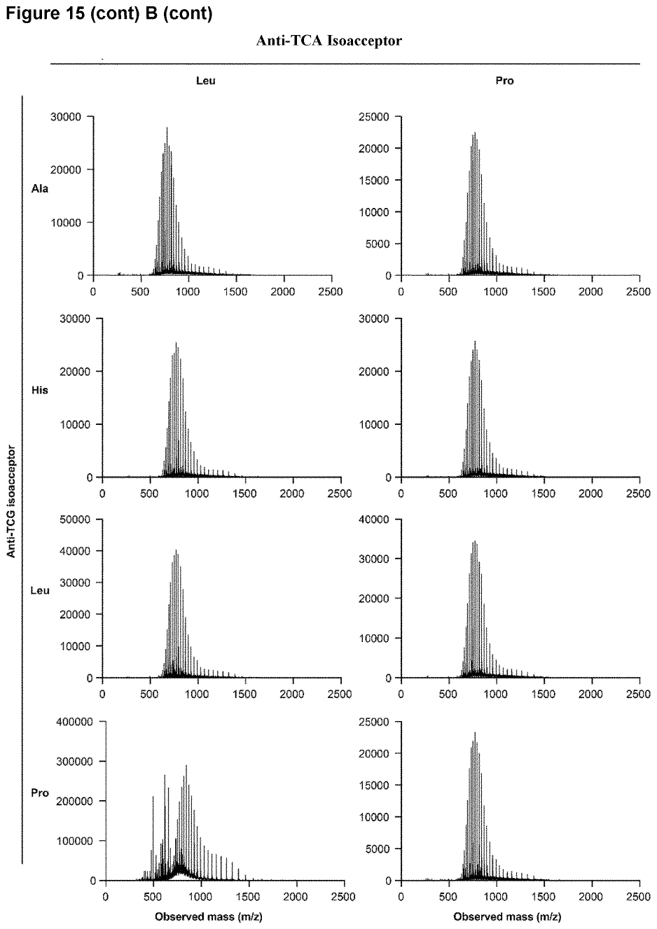

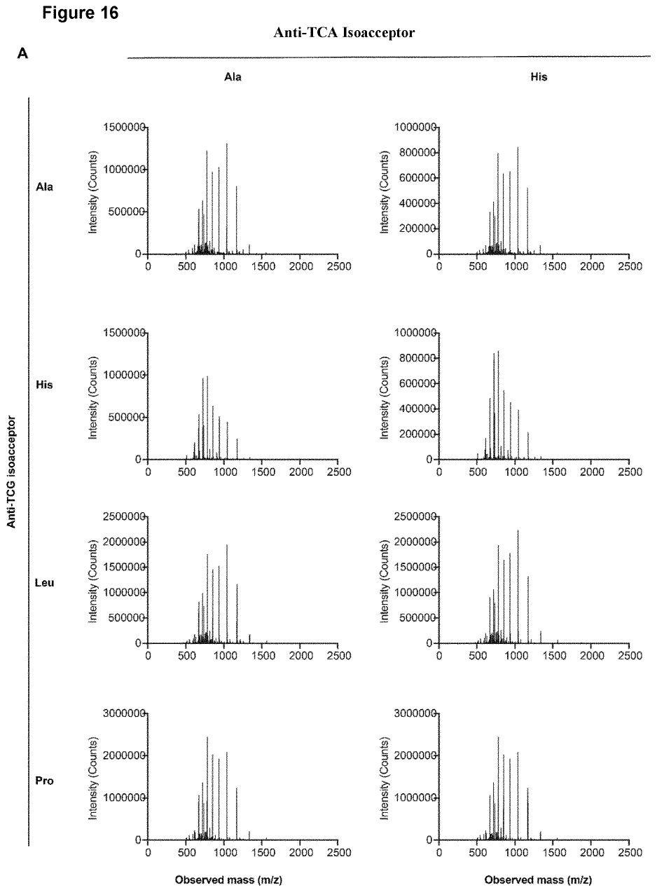

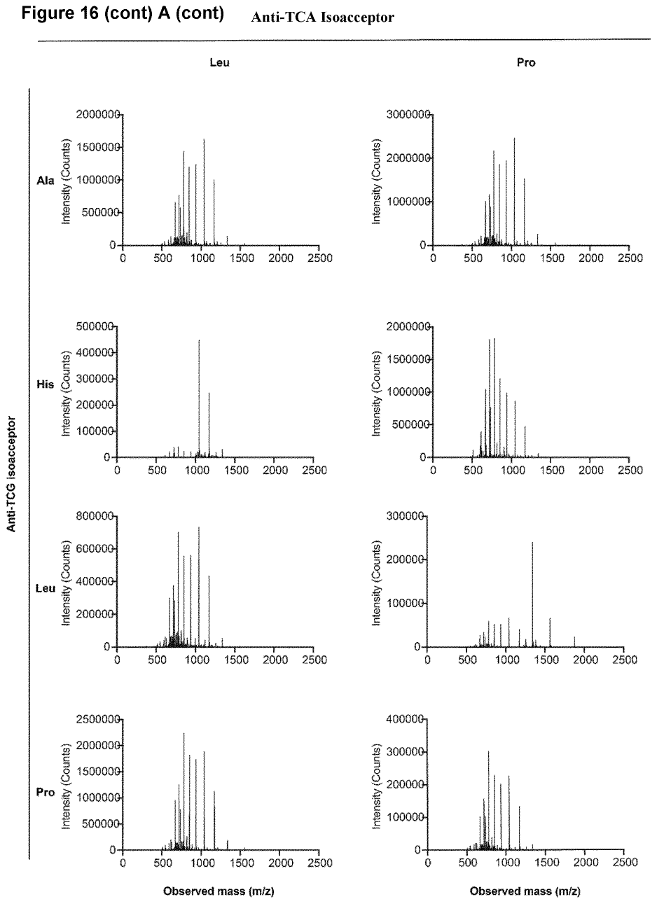

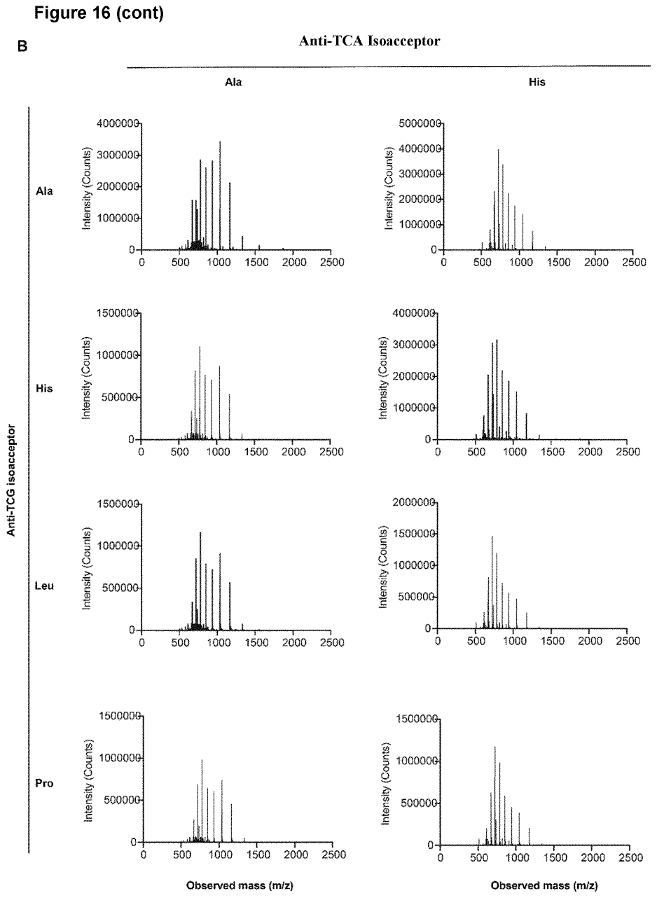

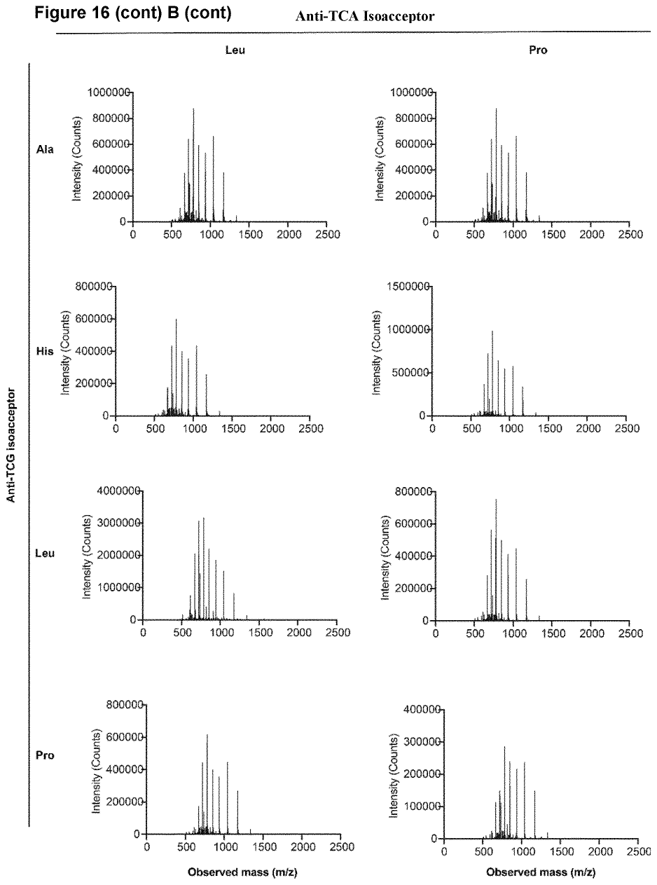

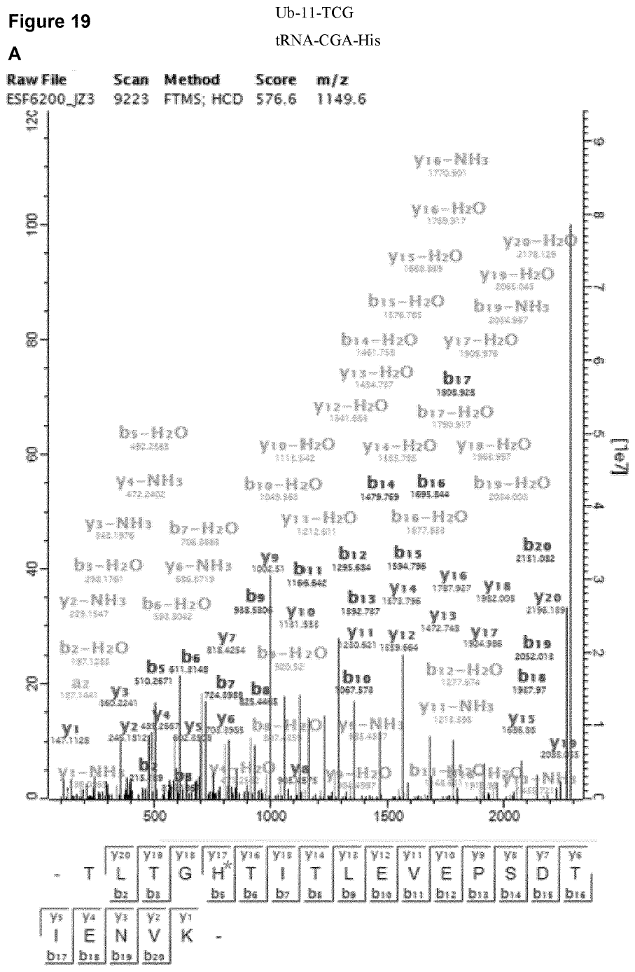

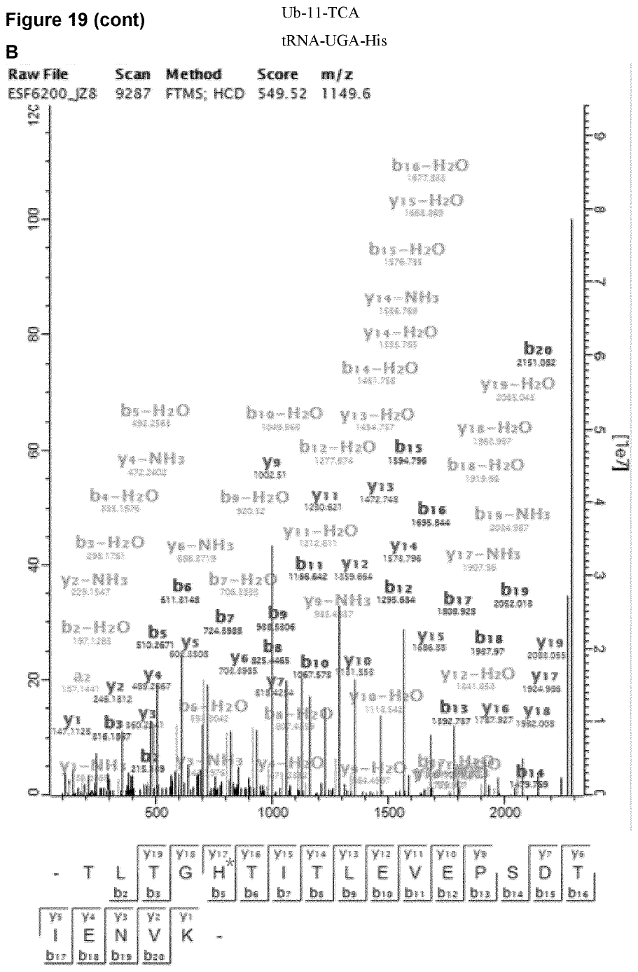

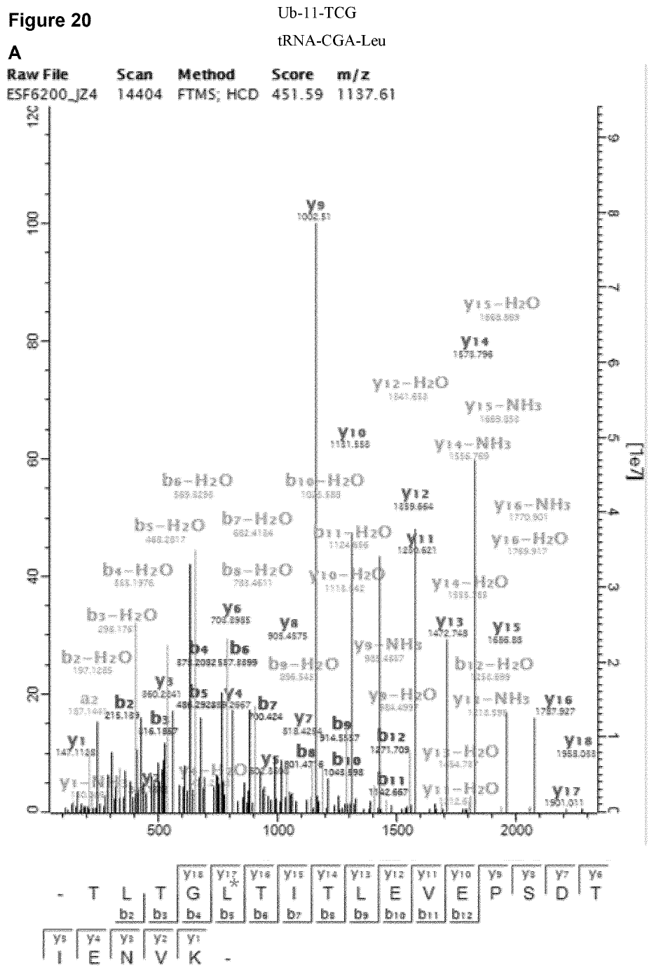

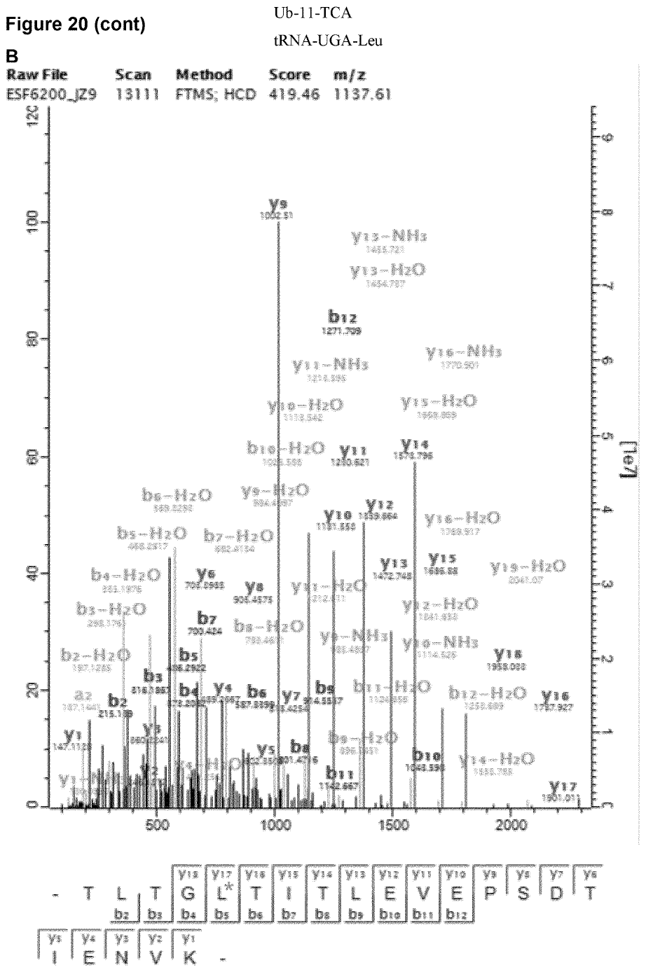

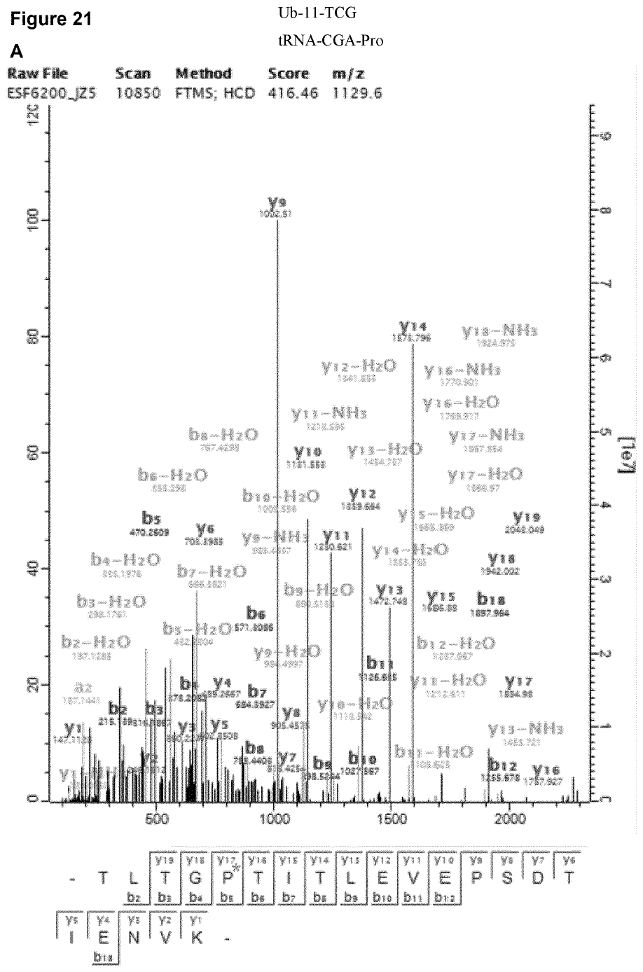

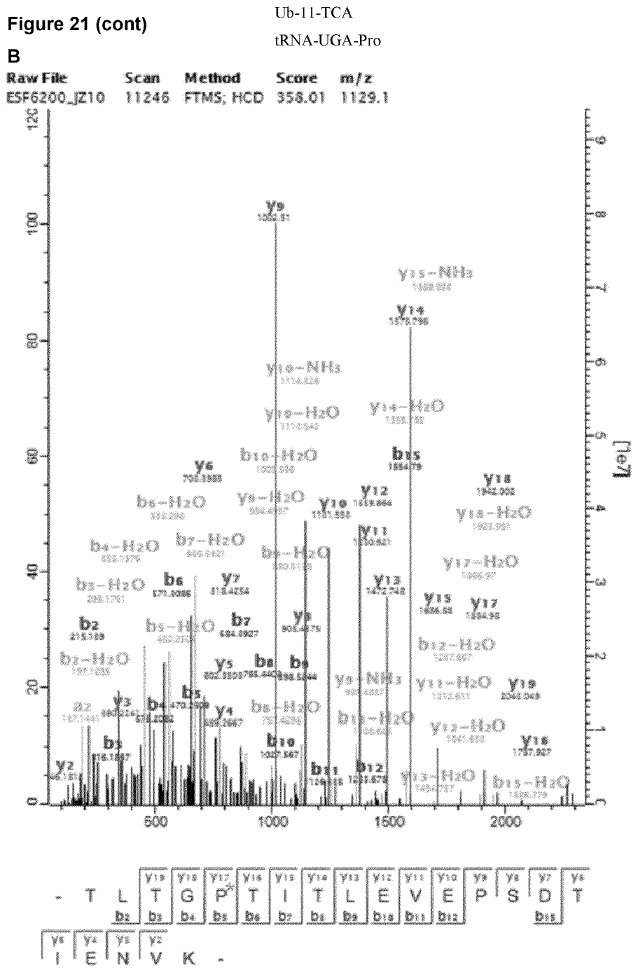

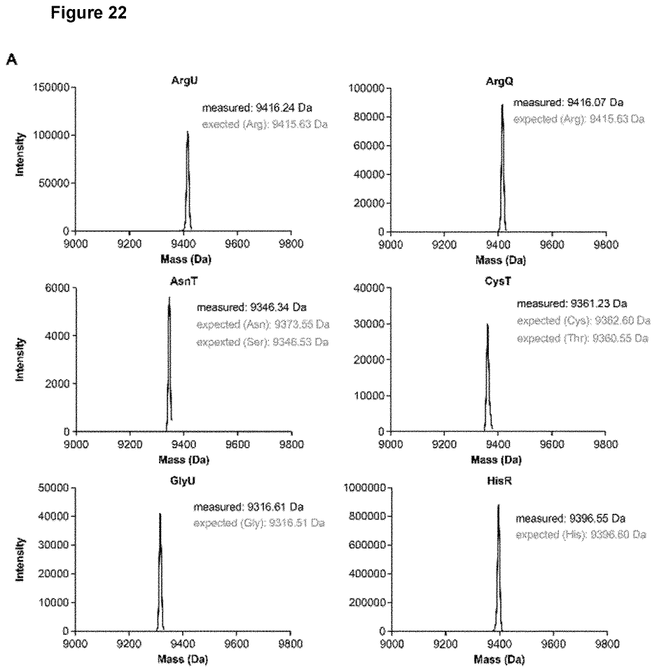

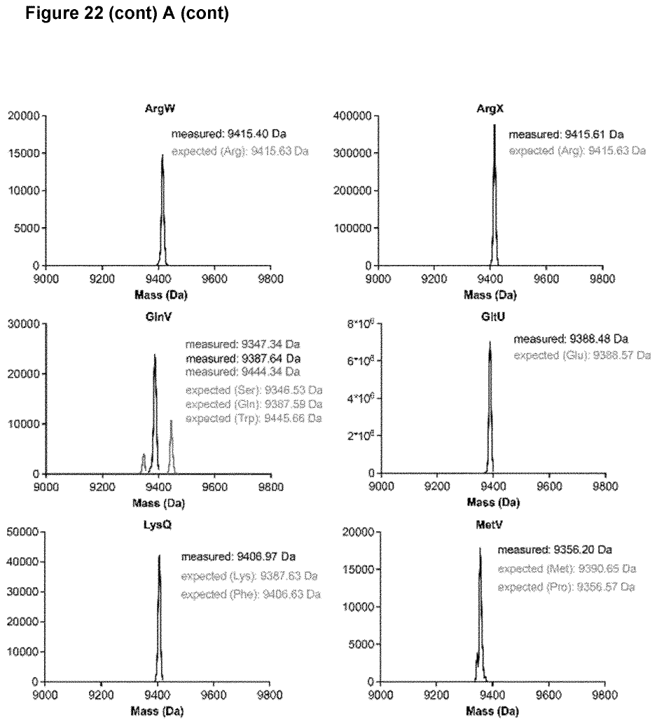

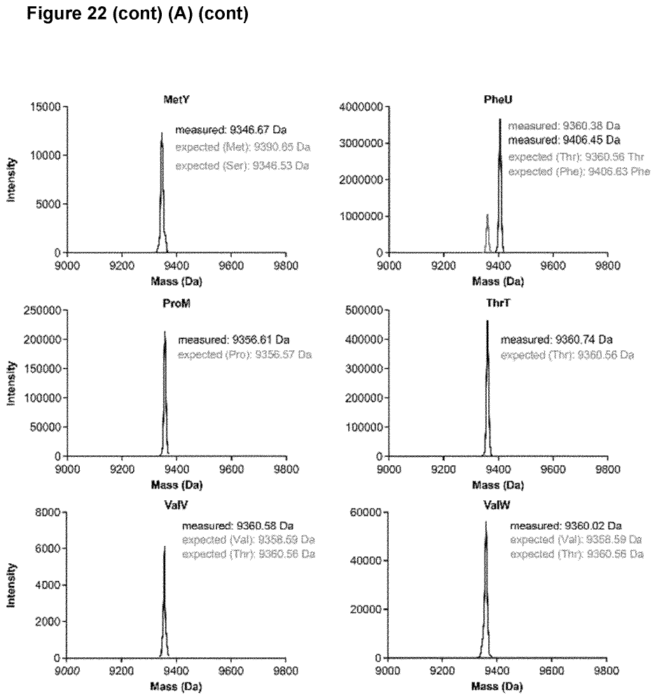

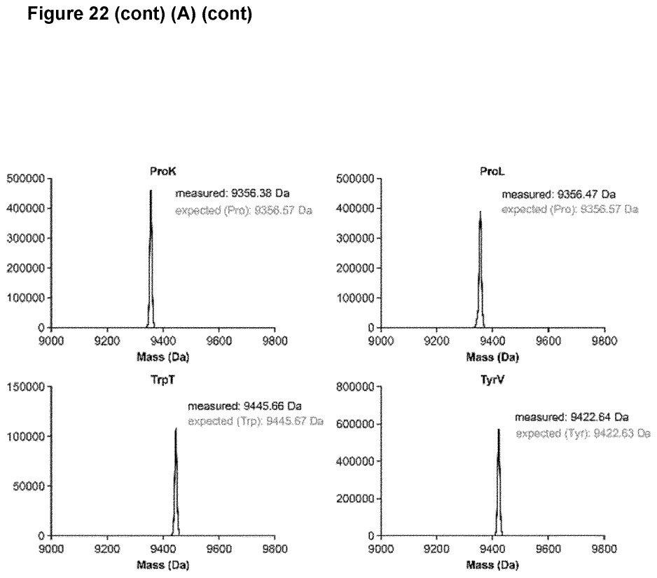

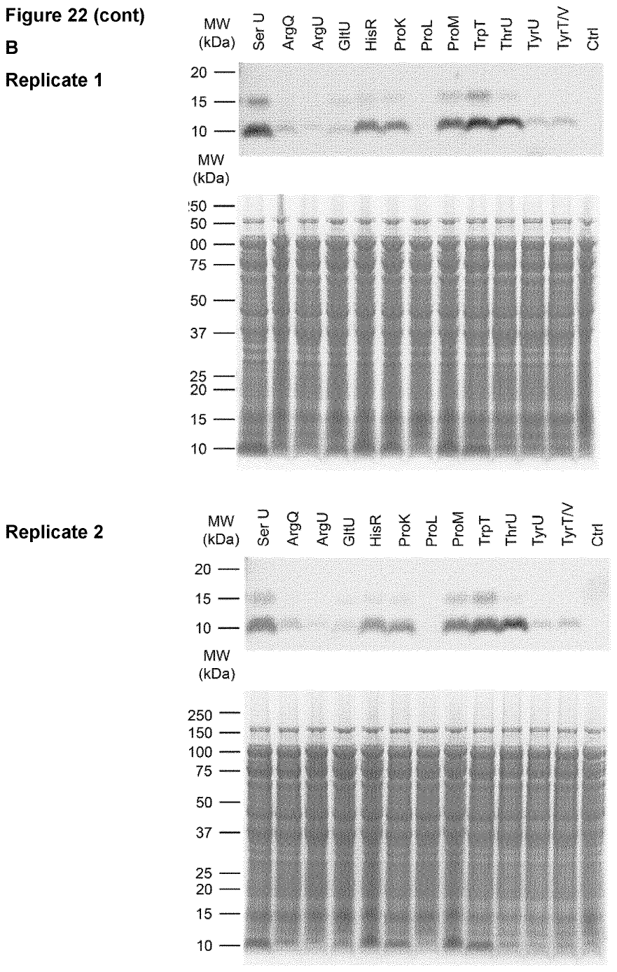

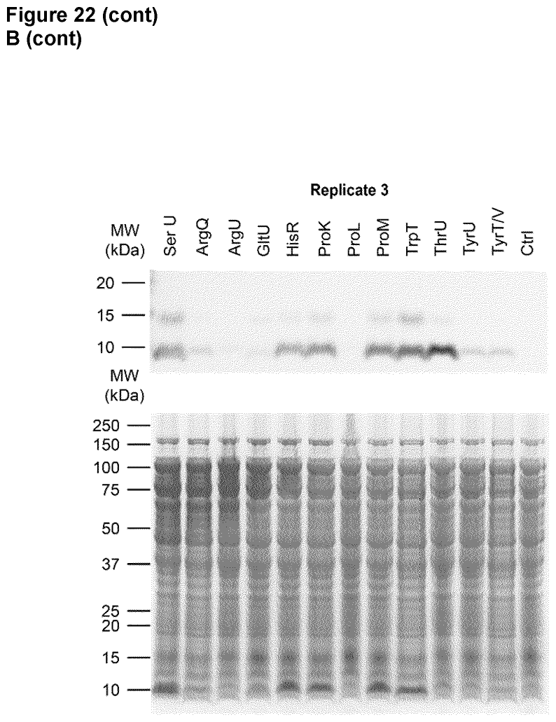

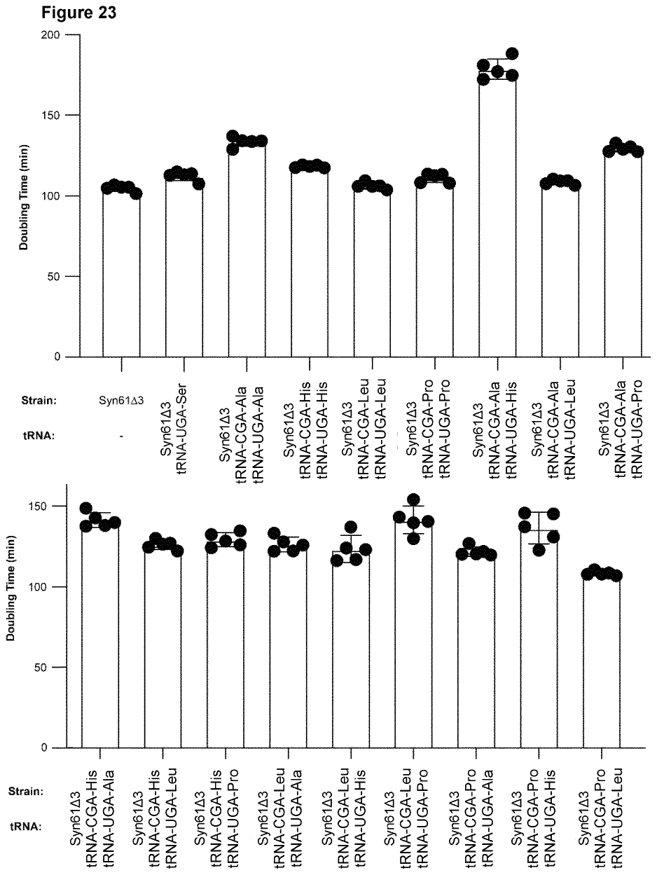

serU, encoding tRNA_{CGA}^{Ser}, and serT, encoding tRNA_{UGA}^{Ser}, both decode TCG and TCA codons and incorporate serine into proteins in Syn61Δ3 (FIG. S3). To reassign the TCG and TCA codons to distinct natural amino acids in Syn61Δ3, we created variants of the isoacceptor tRNAs for canonical amino acids; for each isoacceptor we altered the anticodon to CGA or UGA. We measured the activity of these chimeric tRNAs for decoding TCG or TCA codons at position 3 of an sfGFP gene (a known permissive site) in Syn61Δ3 (FIG. S3). We found that chimeric tRNAs for Ala (tRNA_{CGA}^{Ala}, tRNA_{UGA}^{Ala}), His (tRNA_{CGA}^{His}, tRNA_{UGA}^{His}), Leu (tRNA_{CGA}^{Leu}, tRNA_{UGA}^{Leu}) and Pro (tRNA_{CGA}^{Leu}, tRNA_{UGA}^{Leu}) specifically direct the incorporation of the amino acid defined by the parent isoacceptor tRNA in response to their cognate codon (TGC or TCA) at position 3 in sfGFP or position 11 in ubiquitin in Syn61Δ3 (FIG. 2, FIGS. 8, 12-21), and produce good yields of protein (FIG. S3). We note that the fidelity of tRNA_{UGA}^{Leu} was lower than that of other tRNAs (FIG. 13). Alanyl and leucyl-tRNAs were investigated since their anticodons are not identity elements for their cognate aminoacyl-tRNA synthetases and they were therefore expected to be permissive to anticodon mutation; the other tRNAs were identified through a screen (FIG. 22). We also found that, unlike tRNA_{CGA}^{Ser} and tRNA_{UGA}^{Ser}, our chimeric tRNAs specifically decode the Watson-Crick complement of their anticodon sequence; for example, tRNA_{CGA}^{Ala} decodes TCG codons in preference to TCA codons and tRNA_{UGA}^{Ala} decodes TCA codons in preference to TCG codons (FIG. S3, FIG. 13). These tRNAs are also specific with respect to other TCN codons (FIG. 14, FIG. 15), and most reassigned strains grew comparably to parental strains (FIG. 23). Therefore, we can independently re-assign the TCA and TCG codons to Ala, His, Leu or Pro in Syn61Δ3 and thereby create 16 new genetic codes (FIG. S3, Data File S1, FIG. 2B). In each new genetic code we changed the identity of the canonical amino acids encoded at specific sense codons with respect to both the canonical code and the other 15 codes we have created (FIG. 2C).

Overall we have refactored the structure of the genetic code. Our new genetic codes expand the number of codons used to encode Ala and Pro (from 4 to 6), double the number of codons used to encode His, from 2 to 4, and an increase the number of codons used to encode Leu from 6 to 8; this is more codons than are used to encode any amino acid in the canonical code. These experiments also show that the UCN codon box, which encodes serine in the canonical code, can be split to encode additional canonical amino acids.

### Example 4—Orthogonal Code-Orthogonal Decoder Pairs

Genes written using the canonical genetic code, in which TCG and TCA codons encode serine, will make the correct protein product in natural cells that read these codons as serine. However, these genes will yield the incorrect—likely non-functional—protein product in cells that decode these codons to incorporate amino acids other than serine.

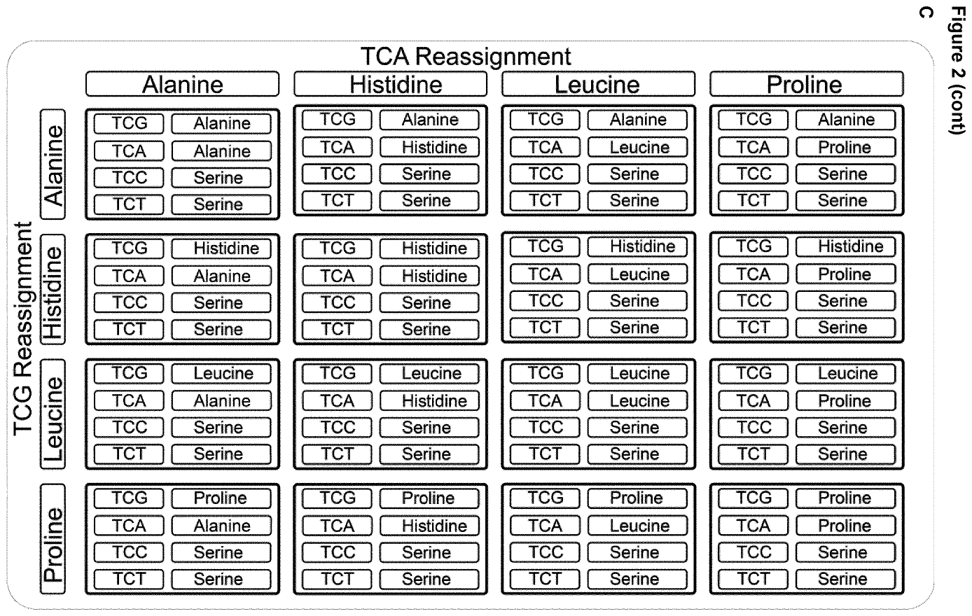

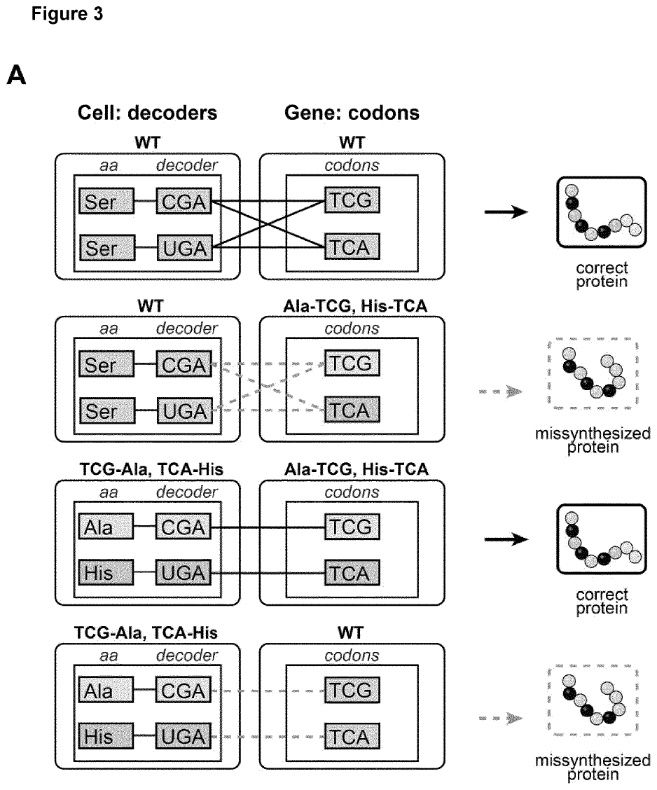

Similarly, synthetic genes—in which we compress the genetic code using the Syn61 recoding scheme and replace codons for specific natural amino acids with TCG and TCA codons—will make the correct protein product in cells that decode the TCG and TCA codons to incorporate the correct amino acid. However these synthetic genes will yield an incorrect—likely non-functional—protein product in cells that read the natural genetic code (FIG. 3).

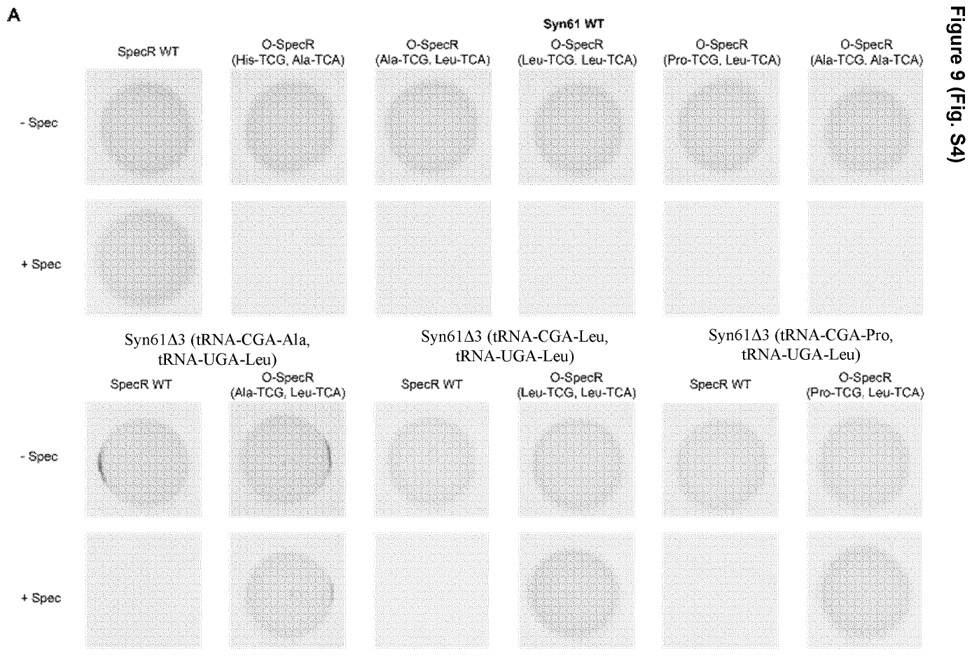

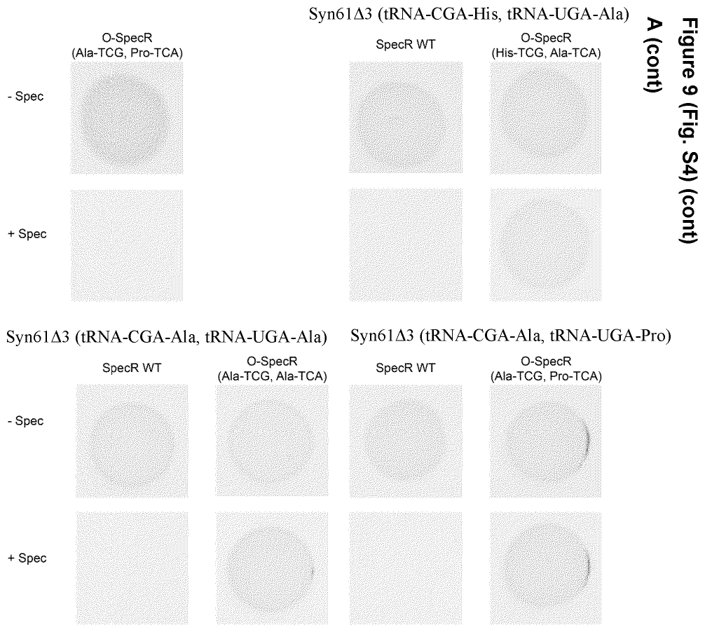

We converted all 27 GCN codons (which encode alanine in the canonical code) to TCG codons, and all 6 CAT/C codons (which encode histidine in the canonical code) to TCA codons in recSpecR (ΔTCG, TCA). This created the orthogonal resistance gene O-SpecR (TCG-Ala, TCA-His). We demonstrated that O-SpecR (TCG-Ala, TCA-His) can be decoded in, and confer spectinomycin resistance to, Syn61Δ3 (tRNA_{CGA}^{Ala}, tRNA_{UGA}^{His}) cells, in which TCG is read as Ala and TCA is read as His. We further demonstrated that O-SpecR (TCG-Ala, TCA-His) did not confer spectinomycin resistance to Syn61 WT cells, in which TCG and TCA are decoded as Ser, as in the canonical genetic code. Finally, we demonstrated that SpecR WT, in which serine is encoded using TCG and TCA codons, cannot confer spectinomycin resistance to Syn61Δ3 (tRNA_{CGA}^{Ala}, tRNA_{UGA}^{His}) cells (FIG. 3). We extended this approach to five other reassignment schemes, as well as to other genes (FIG. 9). We obtained similar results (FIG. S4B) with a wt hygromycin resistance gene, and a hygromycin resistance gene in which all Ala codons had been converted to TCG and all histidine codons had been converted to TCA (O-HygR (TCG-Ala, TCA-His)).

These experiments demonstrated that we can create a genetic code-decoder pair for synthetic genes that is functionally orthogonal with respect to the canonical genetic code-decoder pair for natural genes. The orthogonal code (TCG-Ala, TCA-His), written in synthetic genes, is correctly read by the orthogonal decoder (tRNA_{CGA}^{Ala}, tRNA_{UGA}^{His}), but not by the canonical decoder (tRNA_{UGA}^{Ser}). The canonical code (TCG-Ser, TCA-Ser), written in natural genes, is correctly read by the canonical decoder, but not by the orthogonal decoder.

The functional orthogonality of genes in cells with altered decoders will depend on the frequency of reassigned codons and the functional consequences of codon reassignments. The consequences of amino acid substitutions—a result of codon reassignment—may globally, and crudely, correlate with differences in amino acid polarity and hydrophobicity (6, 21). The consequences of amino acid substitutions at particular sites in proteins may be predicted using computational approaches that leverage evolutionary sequence- and/or structural-information (22-25). While the composition of natural genes is fixed, the codon usage in synthetic genes—written in the standard code or any orthogonal code—can be simply designed to maximize the number of codons subject to reassignment, and thereby maximize the functional orthogonality of synthetic genes.

### Example 5—Orthogonal Horizontal Gene Transfer

Next, building on orthogonal genetic code-decoder pairs, we created orthogonal horizontal gene transfer (O-HGT) systems composed of an orthogonal decoder and a mobile genetic element that uses an orthogonal genetic code. WT cells can transfer a WT mobile genetic element between themselves, but cannot transfer the WT mobile genetic element to cells containing orthogonal decoders. Cells containing O-HGT systems can transfer their mobile genetic element to cells that contain a compatible orthogonal decoder, but cannot transfer their mobile genetic element to cells containing an incompatible orthogonal decoder or to WT cells.

A mobile genetic element (F plasmid, F (WT)), which uses the canonical genetic code was transferred to WT cells (Syn61 WT), as expected. We also showed that F (WT) could not be transferred to Syn61Δ3 (tRNA_{CGA}^{Ala}, tRNA_{UGA}^{His}) cells, in which TCG codons are read as Ala and TCA codons are read as His (FIG. 4).

Next we investigated horizontal gene transfer for a mobile genetic element with an altered genetic code. We synthesized the mobile genetic element O-F1 (TCG-Ala, TCA-His). The genetic code in all annotated open reading frames of this F plasmid was compressed using the Syn61 scheme, and GCN codons (which encode alanine in the canonical code) and CAT/C codons (which encode histidine in the canonical code) were converted to TCG and TCA codons respectively within the trfA gene—this gene is essential for the replication of the mobile genetic element.

O-F1 (TCG-Ala, TCA-His) was horizontally transferred to Syn61Δ3 (tRNA_{CGA}^{Ala}, tRNA_{UGA}^{His}) cells. We further demonstrated that O-F1 (TCG-Ala, TCA-His) was not horizontally transferred to cells which read the canonical genetic code (FIG. 4). These experiments demonstrated that we can create O-HGT systems.

Next we created mutually orthogonal HGT systems, which are orthogonal to the natural genetic system and to each other. We created a new mobile genetic element O-F2 (TCG-His, TCA-Ala). The genetic code in all annotated open reading frames of this F plasmid was compressed using the Syn61 scheme and GCN codons (which encode alanine in the canonical code) and CAT/C codons (which encode histidine in the canonical code) were also converted to TCA and TCG codons respectively within the trfA gene.

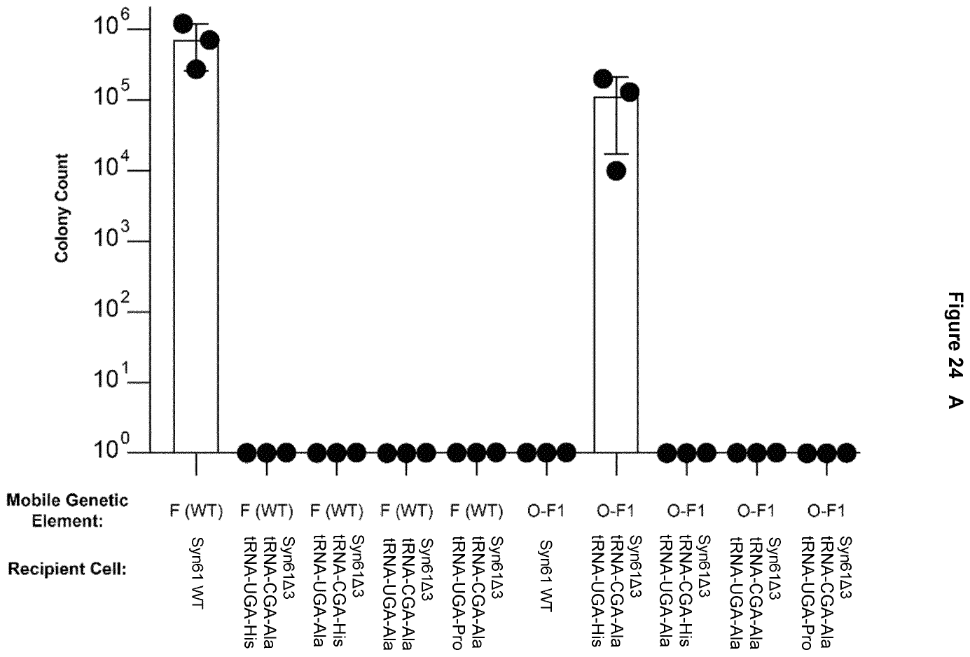

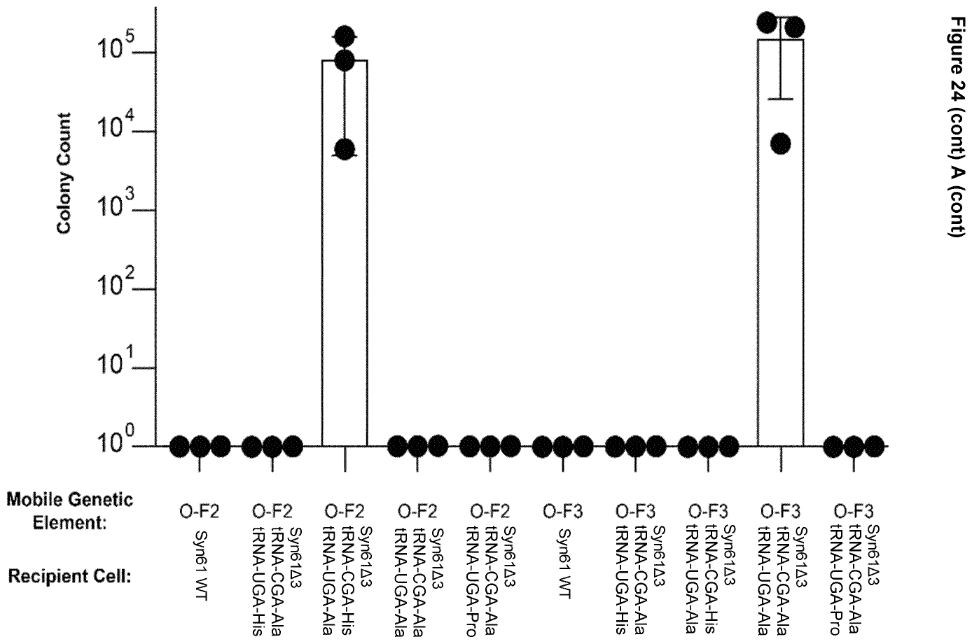

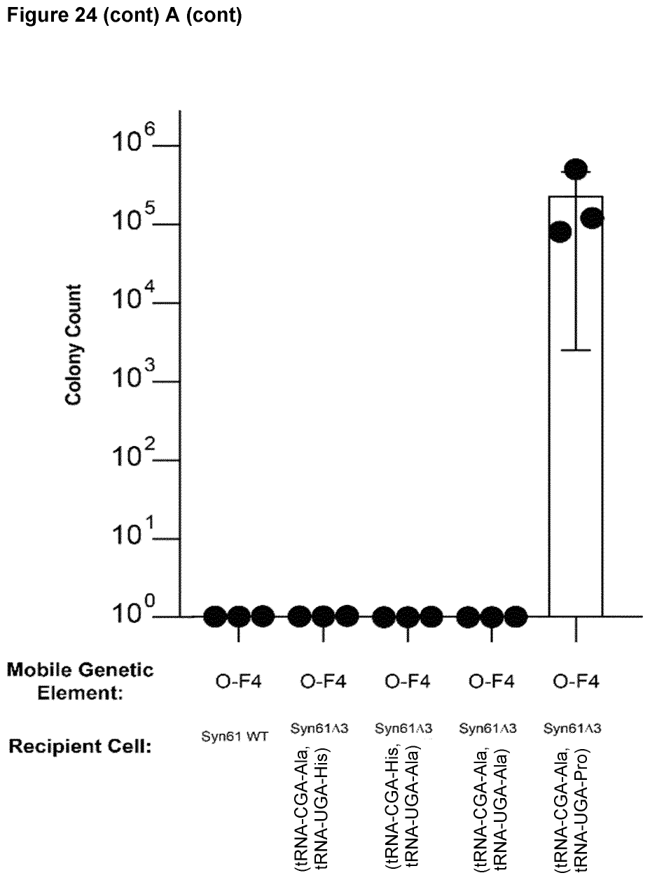

We demonstrated that O-F2 (TCG-His, TCA-Ala) could be transferred to Syn61Δ3 (tRNA_{CGA}^{His}, tRNA_{UGA}^{Ala}) cells, in which TCG is decoded as His and TCA is decoded as Ala. In contrast O-F2 (TCG-His, TCA-Ala) was not transferred into Syn61 cells, which use the canonical genetic code to decode TCG and TCA codons as Ser. O-F2 (TCG-His, TCA-Ala) was not transferred to Syn61Δ3 (tRNA_{CGA}^{Ala}, tRNA_{UGA}^{His}) cells. Moreover, we demonstrated that neither a WT mobile genetic element (F (WT; TCG-Ser, TCA-Ser) nor O-F1 (TCG-Ala, TCA-His) were transferred into Syn61Δ3 (tRNA_{CGA}^{His}, tRNA_{IGA}^{Ala}) cells (FIG. 4). Further experiments demonstrating HGT systems are illustrated in FIG. 24. Overall, we demonstrated the scalability of our approach through the creation of five mutually orthogonal horizontal gene transfer systems.

These experiments demonstrated that we can create orthogonal and mutually orthogonal HGT systems.

### Example 6—Orthogonal Code-Locking Blocks Invading Codes

We hypothesized that replacing codons for specific natural amino acids in essential genes with TCA and TCG codons, and adding tRNAs that reassign these codons to the specific natural amino acids (FIG. 5), would obstruct the serT-mediated HGT we observed into Syn61Δ3 (FIG. 1).

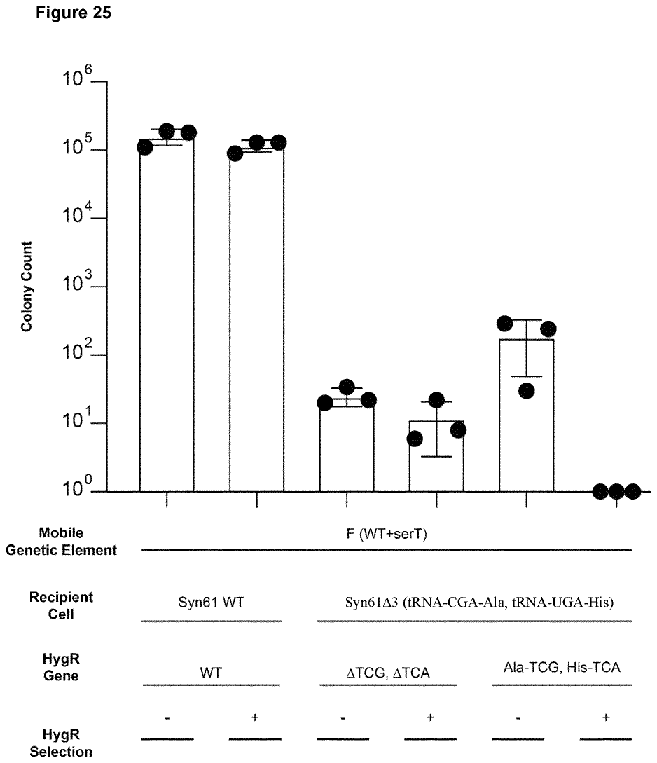

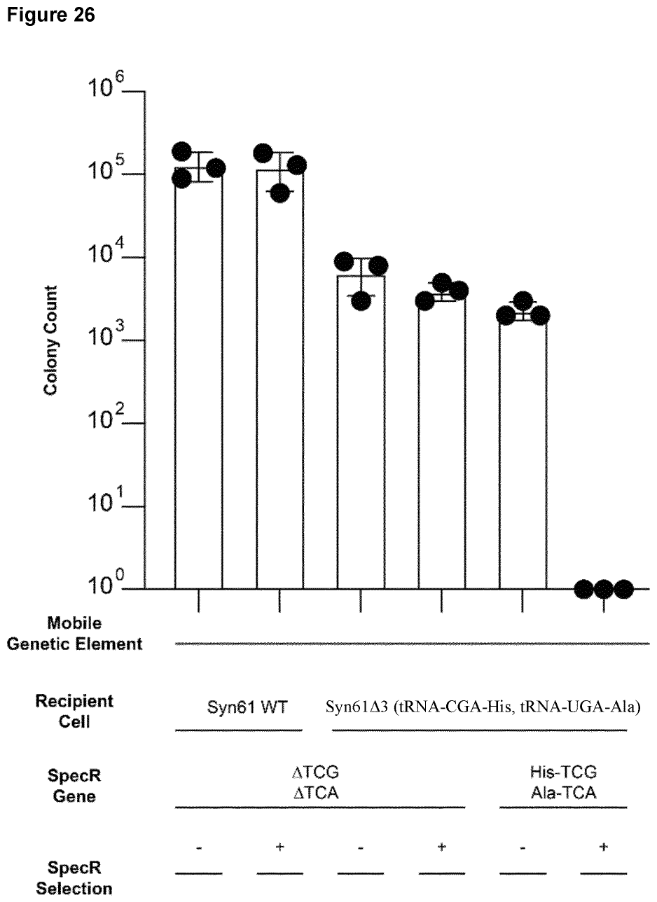

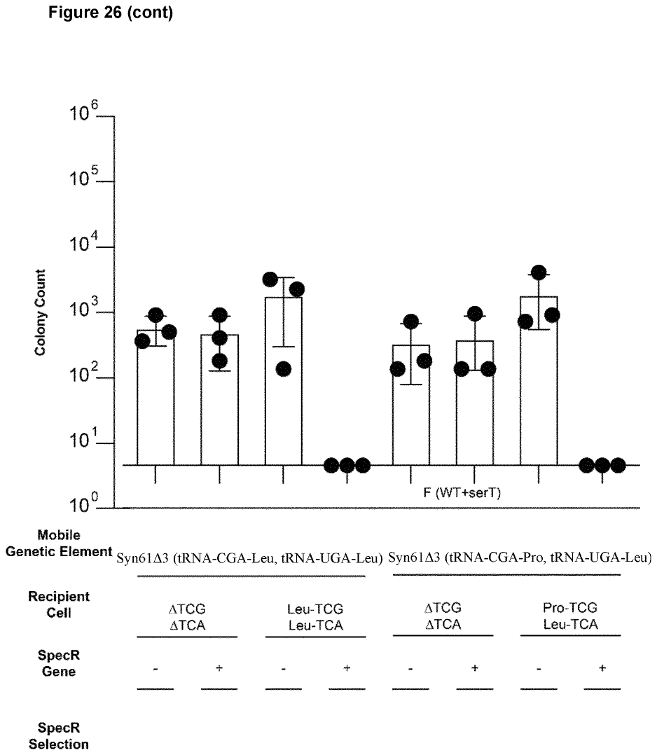

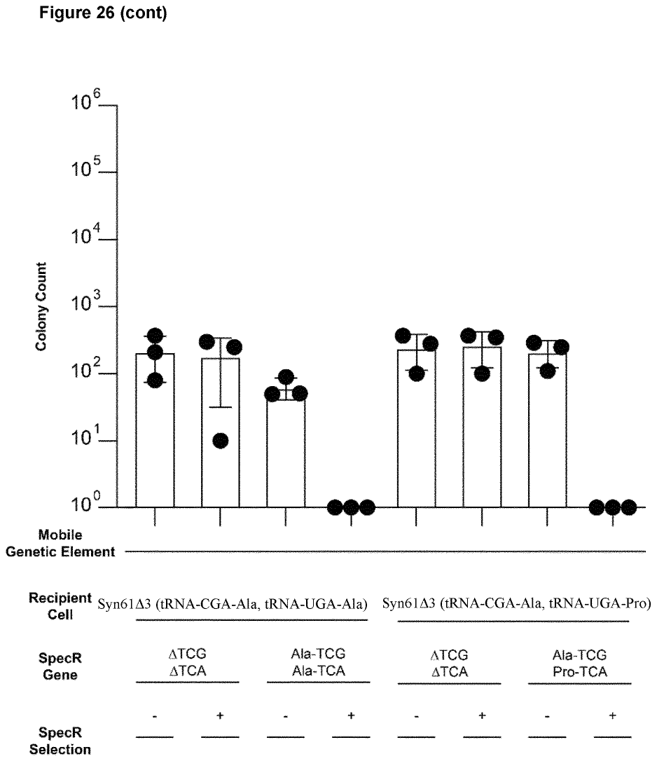

Transfer of F (WT+serT) to Syn61Δ3 (tRNA_{CGA}^{Ala}, tRNA_{UGA}^{His}, O-SpecR(TCG-Ala, TCA-His)) was obstructed (10⁴ fold) in the absence of spectinomycin, as tRNA_{CGA}^{Ala} and tRNA_{UGA}^{His} compete with tRNA_{UGA}^{Ser} in the recipient cell to decrease the production of functional proteins from the mobile genetic element. However, this obstruction was not sufficient to completely ablate transfer of the mobile genetic element. Upon addition of spectinomycin—making O-SpecR(TCG-Ala, TCA-His) an essential gene in the cell—the decoding of TCG codons as alanine and the decoding of TCA codons as histidine become essential. Under these conditions transfer of the mobile genetic element was completely ablated (FIG. 5). Similar results were obtained with other refactored codes and other essential genes (FIG. 25, FIG. 26).

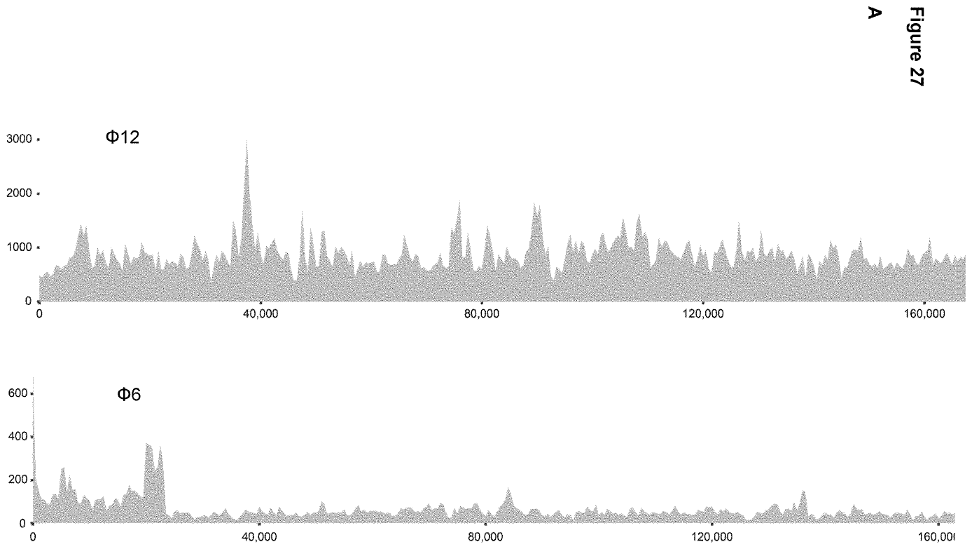

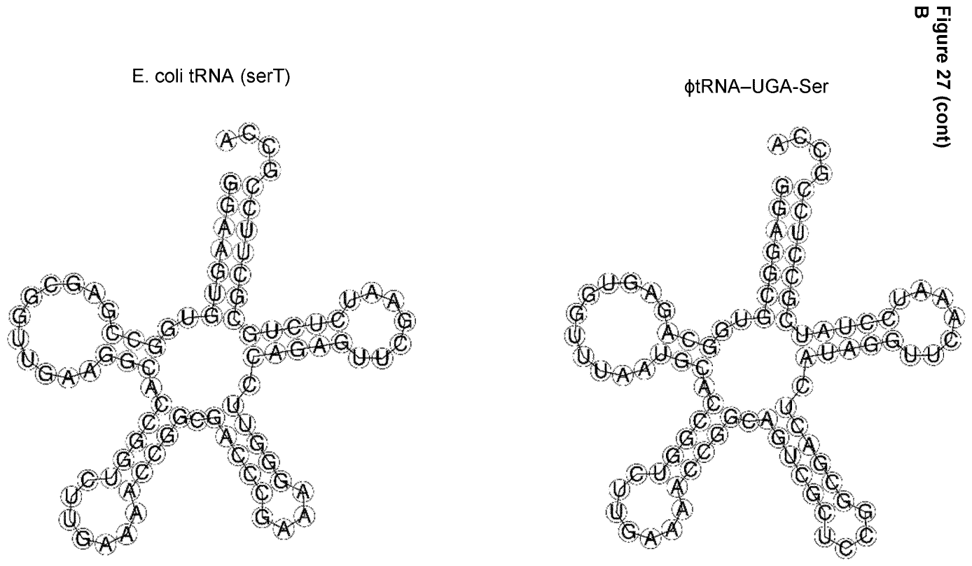

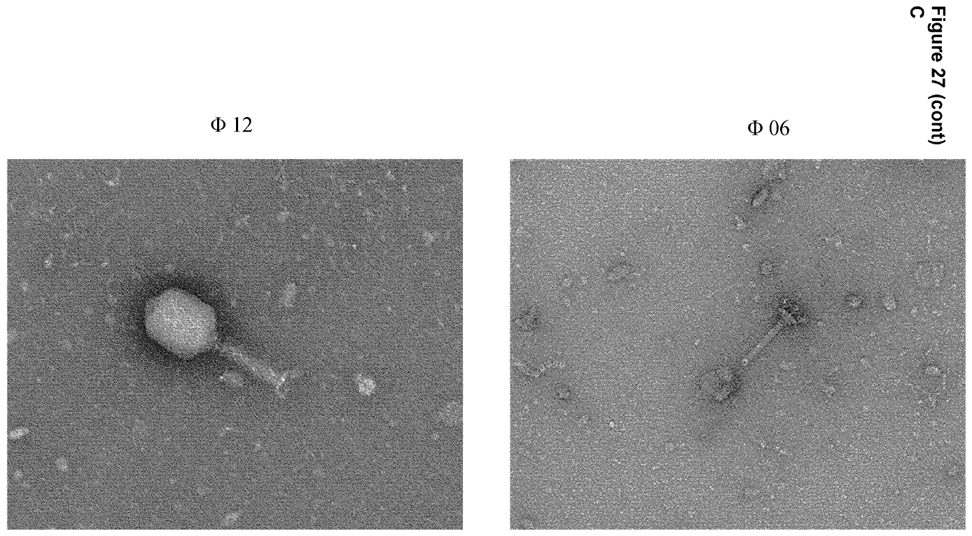

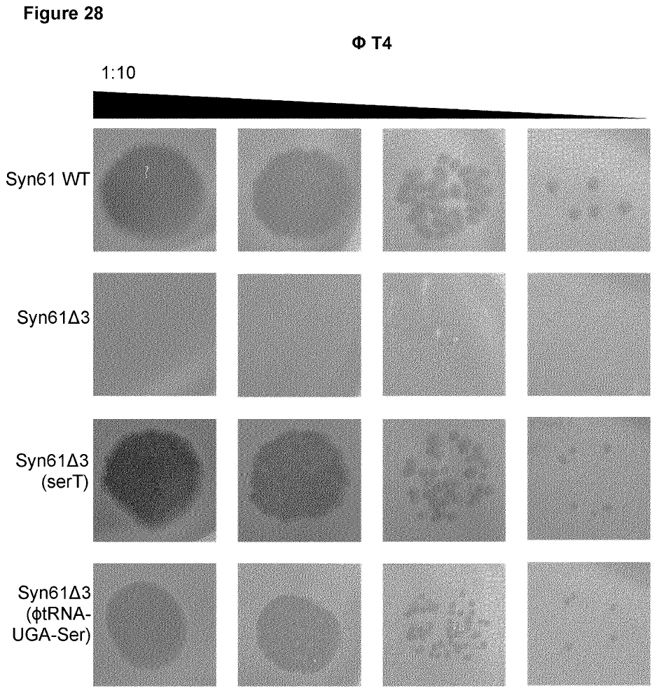

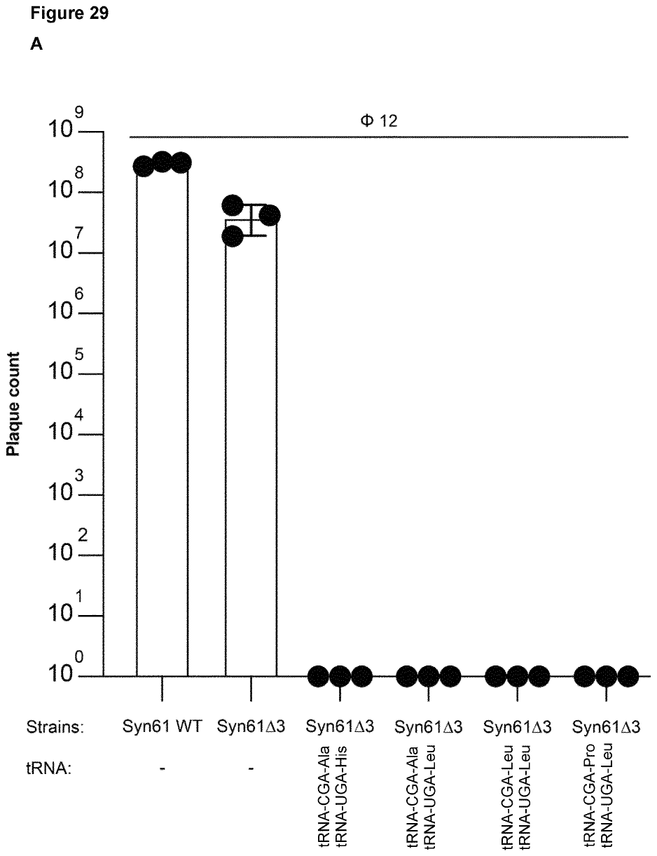

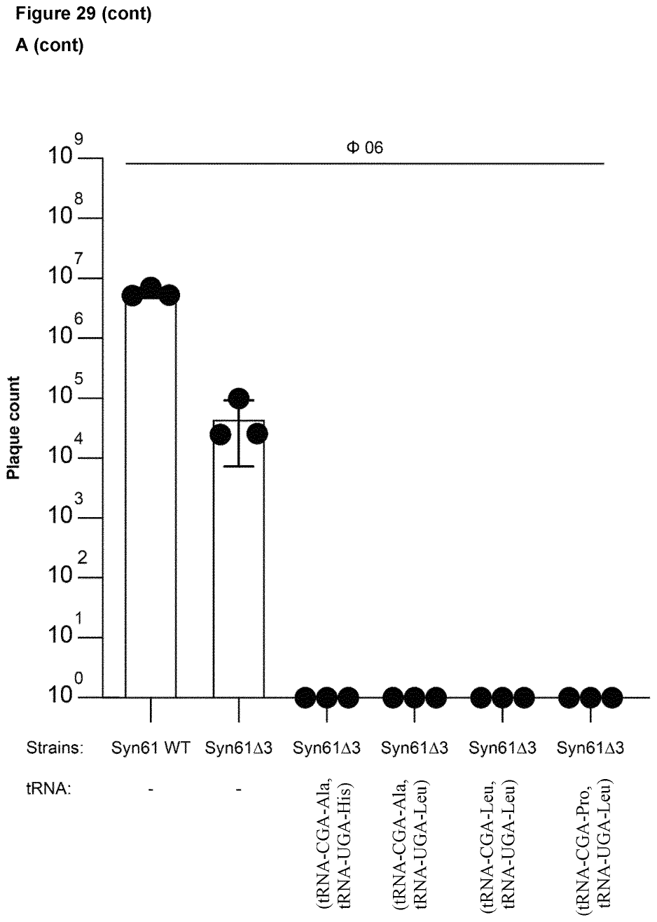

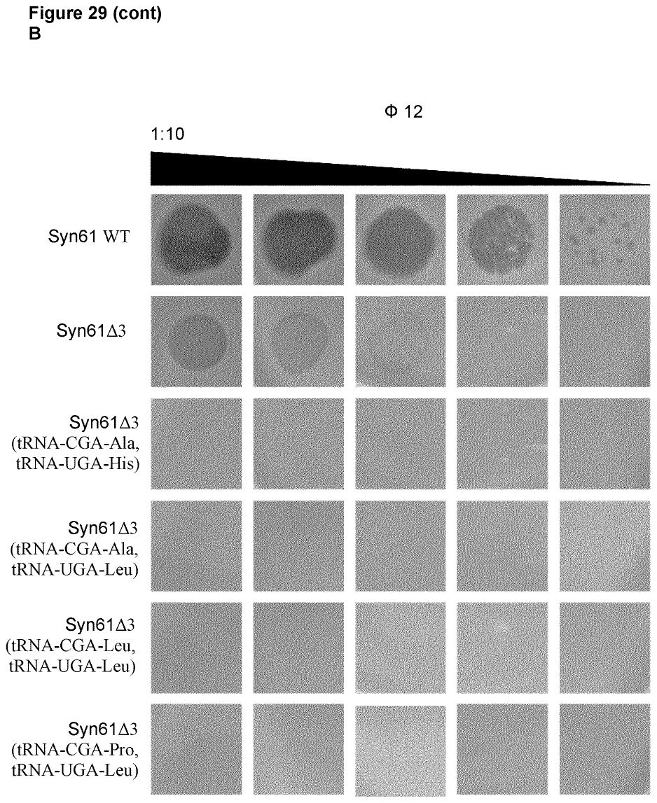

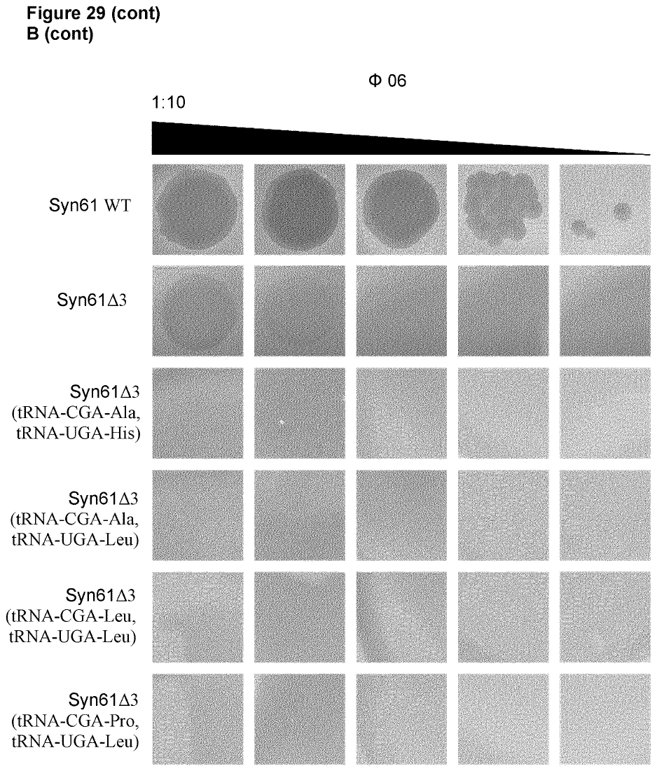

To extend our approach to viral infection, we identified pools of phage from the River Cam that can infect Syn61Δ3 (Methods). From these pools we isolated two individual phage (12 and 06 both T4-like phage), which carry an identical tRNA_{UGA}^{Ser} gene and infect Syn61Δ3 (FIG. 27); some viruses are known to carry their own tRNAs and other translation factors, to augment the cellular pool of translation factors and assist in the translation of codons within their own genes. As expected, expression of this tRNA in Syn61Δ3 is sufficient to confer susceptibility to infection by (otherwise non-infectious) T4 phage (FIG. 28). We demonstrated that, unlike Syn61Δ3, several refactored, code locked strains were completely resistant to infection with phage 6 and phage 12 (FIG. 5, FIG. 29).

Our results demonstrate that writing essential genes in an orthogonal code and reading these genes with a cognate orthogonal decoder creates cells that are locked into the orthogonal code. These cells that resist invasion by mobile genetic elements that use competing codes.

### Example 7—Genetic Code-Locking Enables Stable Phage Resistance in a Synthetic Organism

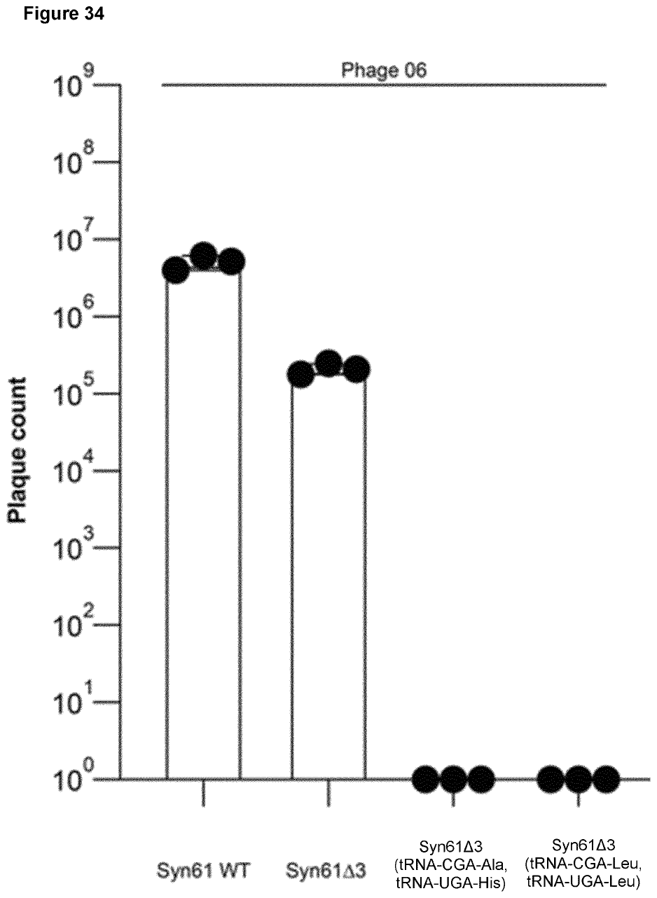

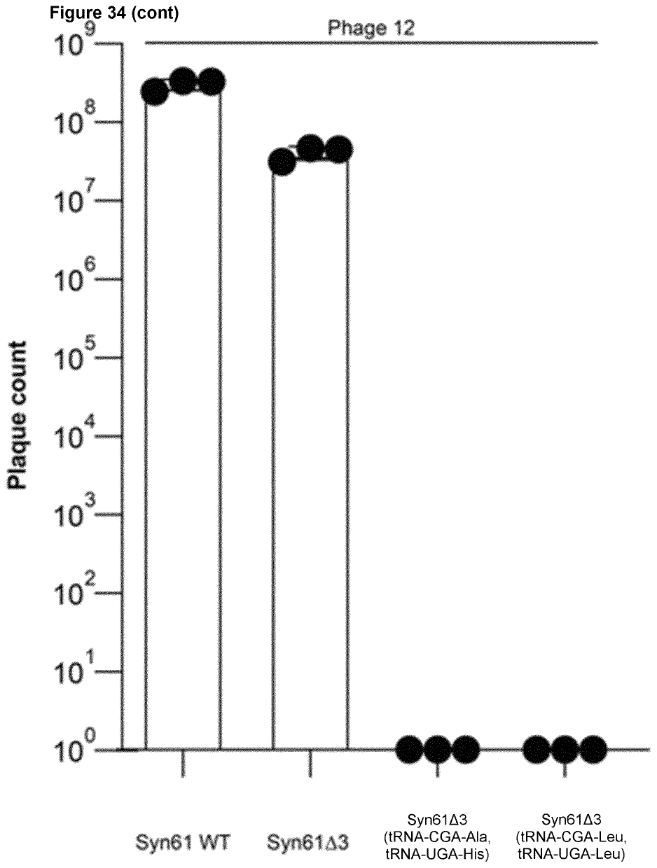

The experiments discussed in the preceding Examples, identify phage from nature that encode for a seryl-tRNA (tRNA^{Ser}_{UGA}) on their genome and showed that such phage can infect Syn61Δ3. These experiments also showed that we could ablate infection by these phage through code-locking (FIG. 34). However, we also show that, in contrast to conjugative transfer, reassignment is sufficient to ablate plaque formation. Further experiments were performed to determine why this the case.

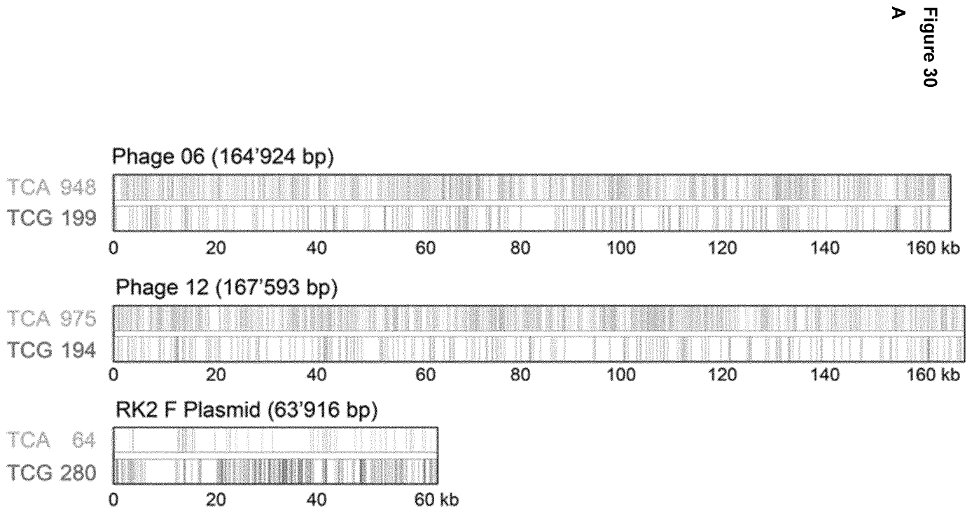

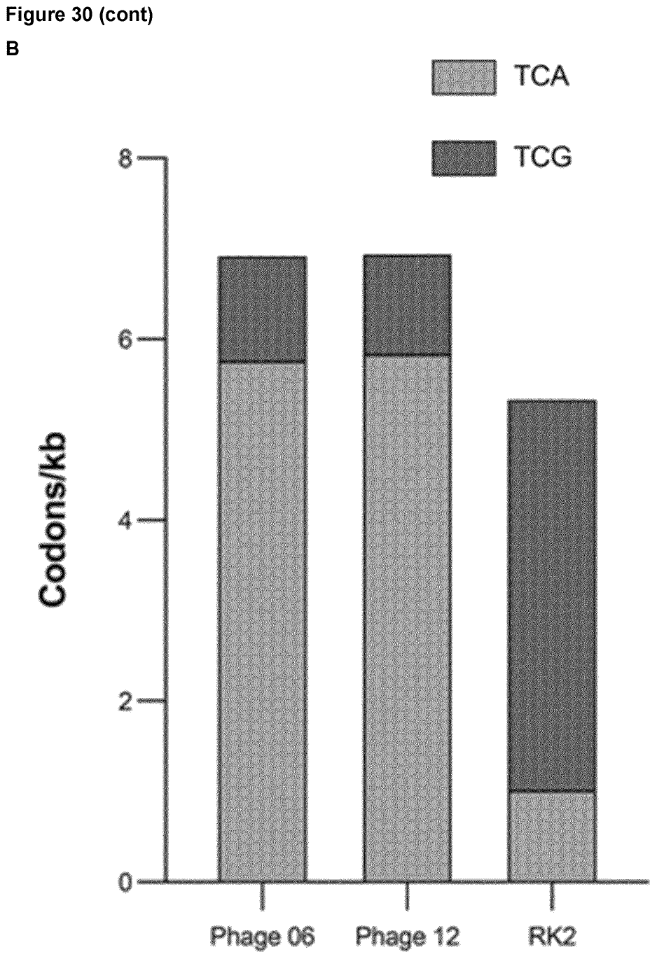

We theorised that genomic differences may explain the phenotypic differences; one possible explanation is the number of TCG and TCA codons in the respective genomes. The genomes of the phage investigated here are considerably larger than F′ (WT+serT) and the total number of target codons is more than three times bigger (FIG. 30a). As more positions are affected by amino acid misincorporation, the chance that there is a deleterious effect is higher.

It could also be the genomic frequency of target codons. In comparison to F′ (WT+serT) the phage genomes show an about 25% increased frequency of target codons in their genome (FIG. 30b). With increased frequency of amino acid misincorporation the chance that an open reading frame is translated non-functionally is higher.

These two effects may add up. In the phage genomes there are not only more genes affected but these genes are on average affected to a larger degree. Therefore, we would expect codon reassignment to have a bigger impact on phage infection than on conjugative transfer. While it could in principle also be the relative usage of TCG and TCA codons in the genomes of phage 12, 6 and F′ (WT+serT), we think this is unlikely, because reassigning both these codons to leucine leads to the difference between phage infection and conjugative transfer.

It may be that mechanistic differences may explain phenotypic differences. The two modes of horizontal gene transfer, conjugative transfer and phage infection, are fundamentally different processes. For successful phage infection and plaque formation the whole life cycle of the phage needs to be completed. This includes attachment to the cell, injection of the viral genome, production of viral protein, phage genome replication, and maturation of phage particles (FIG. 30c). Only when mature phage particles are formed and successfully infect neighbouring cells can plaques be formed. Completion of the whole life cycle is a highly complex process that requires many parts to work together in tight temporal regulation. For T4-like phages, such as phage 12 and 6, multiple genes are essential for this process. A defect in one of these genes leads to ablation of plaque formation.

In contrast conjugative transfer and subsequent colony formation is a much simpler process. Following the attachment of a donor cell to a recipient, ssDNA is transferred to the recipient through a mating channel. In the recipient cell the DNA is then recircularised to form a stable dsDNA plasmid. Importantly, all proteins involved in this process are either expressed form the recipient cell genome or transferred from the donor alongside the DNA. Subsequently, solely the replication of the plasmid and its proper segregation during cell division needs to be ensured for successful colony formation (FIG. 30d). This process requires very few genes from the conjugative element to be functionally expressed.

Since more genes need to be functionally expressed from horizontally transferred DNA for the successful formation of plaques, it is expected that ambiguous decoding through codon reassignment is more detrimental for phage infection. If one essential gene is disrupted enough for its product not to be functional, the formation of plaques is ablated.

A further explanation is a dominant negative effect arising from ambiguous decoding of certain genes. Dominant negative effects are more likely for proteins that form complex interactions. Some mutations in viral envelope proteins are known to show dominant negative phenotypes. Mutation in a subunit of the envelope presumably interrupts oligomerisation and correct particle assembly. In T4-like phages the major capsid protein (gp23) forms hexamers that are the basis for particle assembly. In phages 06 and 12 there are three surface exposed serine residues that are encoded by TCA (FIG. 32). Amino acid misincorporation at one of these positions in a subset of gp23 could disrupt capsid assembly and thereby have a dominant negative effect on plaque formation.

We realized that an advantage of code locking might be in maintaining the alternative genetic code over time. The tRNAs responsible for code refactoring could be inactivated through a variety of mechanisms, such as mutation, deletion, or silencing. This would essentially revert the cell with a refactored genetic code back to codon compressed cell with decoder deletion (like Syn61Δ3) and render it susceptible to infection by phage that carry a suitable tRNA gene. If the code is locked however, the tRNAs responsible for refactoring are essential and cannot be inactivated. This ensures the temporal stability of the refactored code and with-it phage resistance.

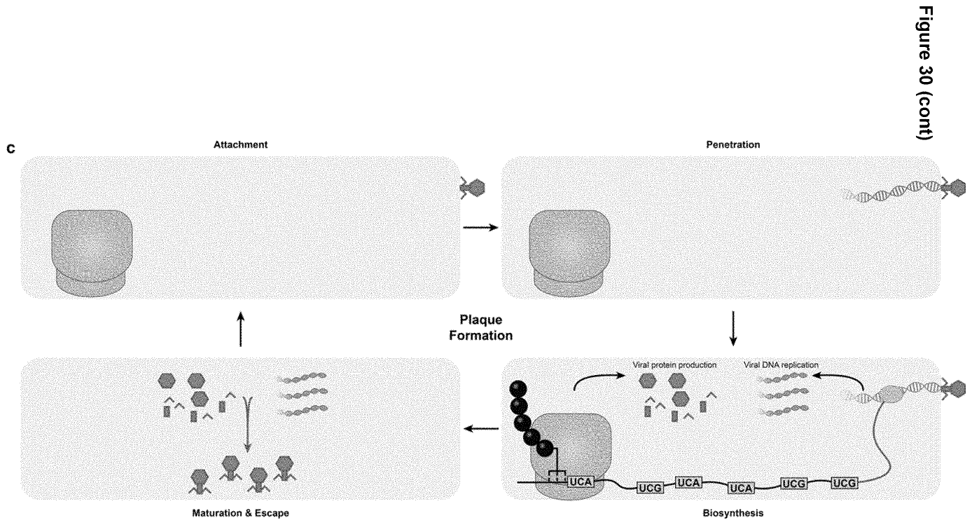

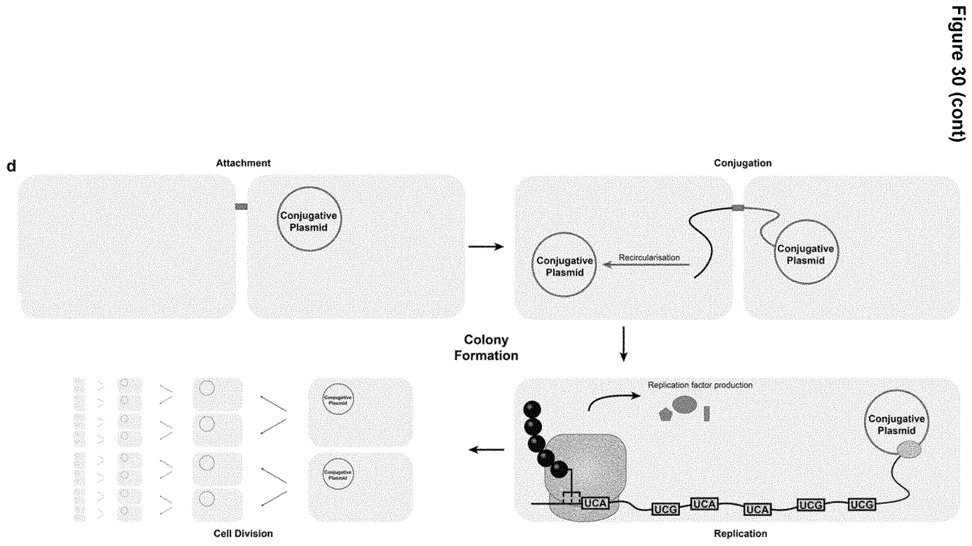

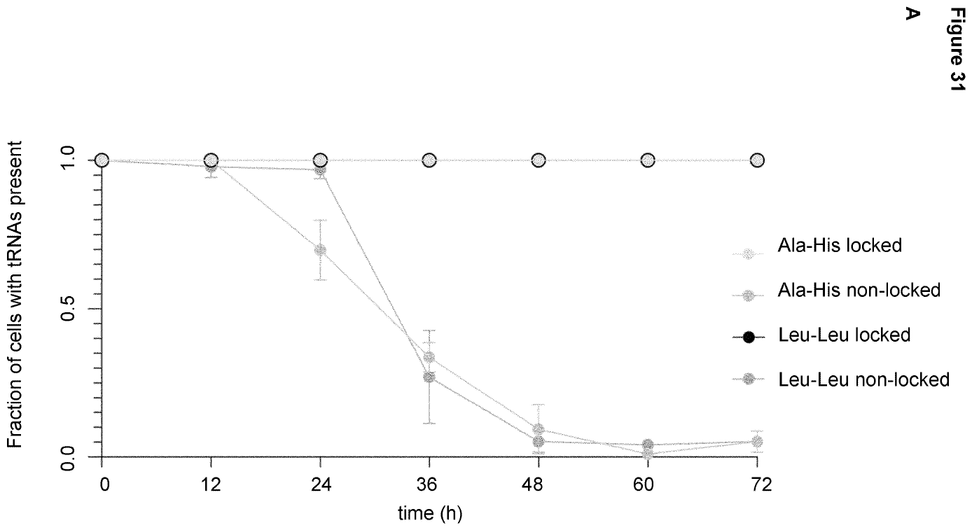

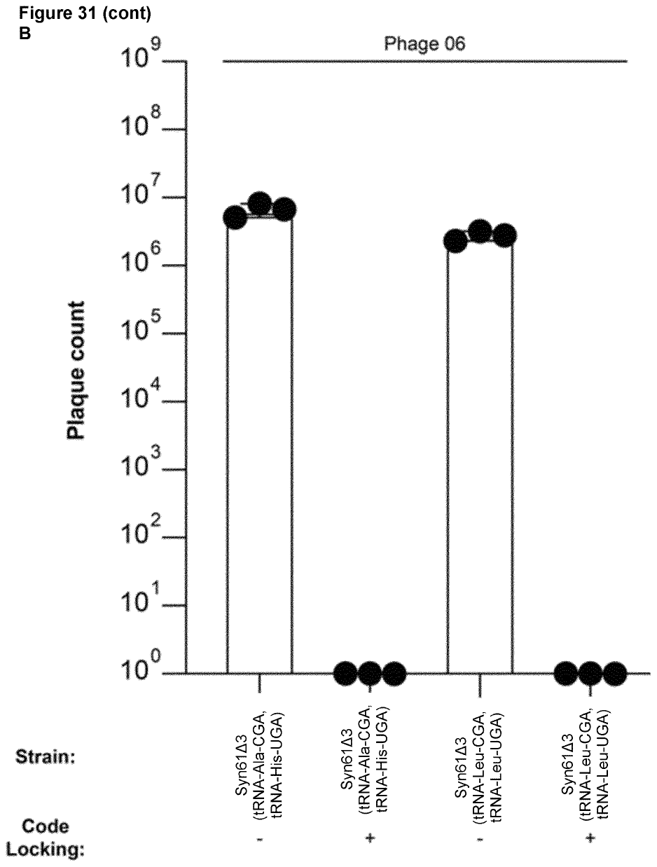

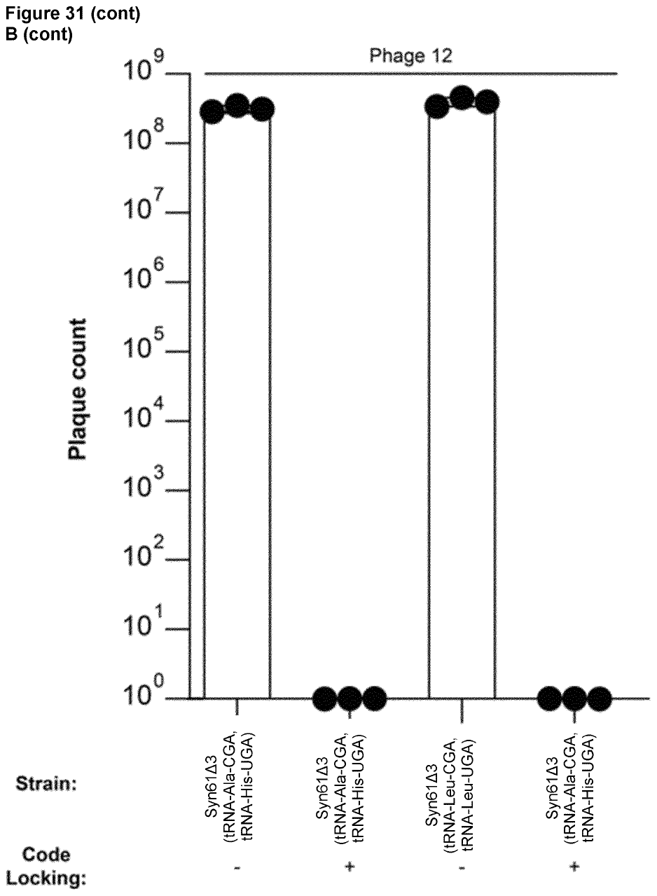

We modelled the stability of alternative genetic codes in the presence and absence of code locking. tRNAs responsible for the alternative decoding of TCG and TCA codons were encoded on a low-copy plasmid bearing a hygromycin resistance. A second plasmid encoded for a variant of a spectinomycin resistance gene (SpecR); For cells without code locking: recSpec^{R} (ΔTCG, ΔTCA), for cells with code-locking: oSpec^{R} (TCG: Ala, TCA: His) and oSpec^{R} (TCG: Leu, TCA: Leu) respectively. Cells were serially passaged in the presence of spectinomycin and absence of hygromycin (no intrinsic pressure to maintain the plasmid). In each passage we measured the fraction of cells that maintained the plasmid encoding the tRNAs. We find that code locking stabilizes alternative codes and acts to maintain the code (FIG. 31a).

Consequently, code locked cells retain phage resistance over time, while non-locked cells lose resistance. We exposed cells with and without a locked code from the time course described above to phage 12 and 06. We observed that cells with a locked genetic code retain resistance to phage infection, while cells that lost the tRNAs responsible for code refactoring are susceptible to phage infection (FIG. 31b).

These experiments also show that a plasmid can be stably maintained by making it essential to the host based on the genetic code. E.g., due to the tRNAs on the plasmid and their necessity to decode an essential gene. This could find utility in avoiding antibiotic remittances in biotech applications.

### Example 8—Phage Propagation Assay

Cells from overnight cultures were diluted to an OD₆₀₀ ˜0.3 and inoculated with phage 12 (MOI=0.001). After 24 h incubation in a volume of 3 mL (2×ty) the phage titre was assessed by serial dilution (7.5 uL spots on a layer of top agar) and plaquing assays on a permissive strain (Syn61 WT). The control was empty media (2×ty) where no cells were present. The detection limit for this assay is at 1 plaque per 7.5 uL (133.3 PFU/mL).

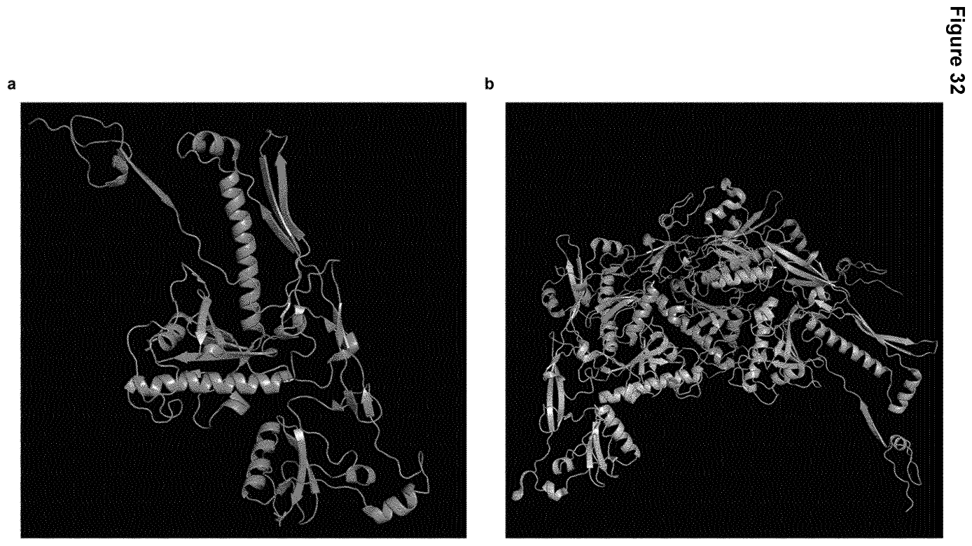

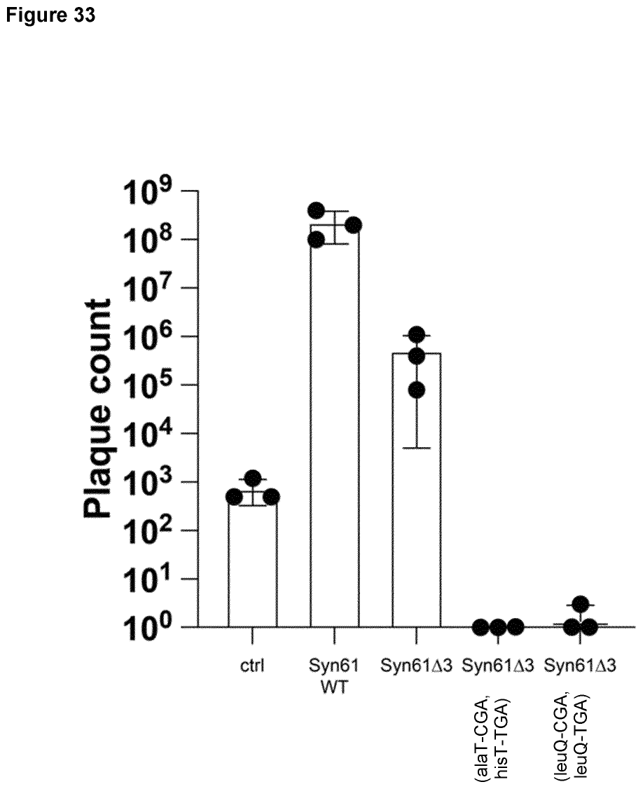

The results are shown in FIG. 33 and show that phage 12 successfully replicates in Syn61WT and Syn61Δ3 cells but not in cells that have refactored and locked genetic codes (Syn61Δ3 (alaTCGA, hisRtga) and Syn61Δ3 (leuQcga, leuQtga)). Compared to the no cell control (ctrl.) phage propagation in code-locked cells yields lower phage titres. This is presumably the case because phage adsorbs to those cells but is unable to replicate. Experiments were performed in biological triplicates.

### Discussion

We have created 16 synthetic genetic codes; in each new code a subset of sense codons are reassigned to different amino acids than in the canonical code. Code reassignment refactors the structure of the genetic code, and directly alters the number, and types, of amino acids that can be accessed by point mutations (FIGS. S5, S6). Previous experimental work has shown that the choice of synonymous codons in individual genes and viruses can alter their robustness and evolvability (26-28), but such approaches are limited to exploring subsets of the canonical code. While a large body of theoretical work, and a limited number of in vitro experiments, have considered the relationship between the structure of the genetic code and its robustness and evolvability (19, 20) it has been impossible to investigate the resulting hypotheses through experiments in living cells. Refactoring the structure of the genetic code provides new opportunities to experimentally test how altered codes affect the robustness and evolvability of protein and cellular function. In future work we aim to leverage genetic code refactoring to accelerate directed evolution.

We have experimentally exemplified the creation of semantic orthogonality between organisms that use distinct reassigned codes. We have explicitly shown that semantic orthogonality creates functional orthogonality for the genes tested; mis-matches between the genetic codes used to write a gene and the decoders used to read the gene leads to mis-synthesized proteins which are non-functional.

We have created multiple mutually orthogonal HGT systems, in which genes can only be correctly read by, and transferred to, cells with cognate decoders. Each type of cell, with a distinct code-decoder system, implements a distinct, refactored, genetic code. These systems may enable experimental investigations into the role of HGT in fixing a universal genetic code, through competition between pools of genotypes written in different codes (4).

Shielding synthetic organisms from environmental genetic elements can be valuable for biotechnological applications on an industrial scale, where contamination with mobile genetic element, including viruses, can cause financial losses and disrupt vital supply chains (29). Resistance to the horizontal transfer of natural genes, into organisms with genomic code compression and tRNA deletion, can be bypassed by re-acquiring the deleted tRNAs, and by mobile genetic elements that carry these tRNAs. Indeed mobile genetic elements—including viruses—carry their own tRNAs and other translation factors, which augment the cellular pool of translation factors and assist in the translation of codons within their own genes (9, 30, 31).

Synthetic organisms with essential genes written in an orthogonal genetic code, and decoders that correctly read the orthogonal code, confer complete resistance to the transfer of mobile genetic elements written in the canonical code, even when the mobile genetic elements contain tRNAs that would allow the cell to correctly read the canonical code. This defines a paradigm for creating organisms that actively resist invasion by foreign codes.

New strategies that limit the transfer of genetic information from synthetic organisms to natural organisms may form the basis of genetic firewalls that isolate synthetic genetic systems from the environment. This is an important challenge, that complements the challenge of controlling the survival and growth of synthetic organisms for biocontainment, especially when considering the use of engineered organisms outside the laboratory (32). All compressed genetic codes are subsets of the natural code and are correctly read by the decoders of the full code; genetic systems written in a compressed genetic code are correctly read by natural organisms. Therefore, compressed genetic codes cannot be used to genetically isolate synthetic organisms from natural organisms. The ability to refactor the structure of the genetic code and write genes that are read correctly in synthetic organisms, but read incorrectly in natural organisms, provides the basis of a powerful strategy to obstruct the transfer of genetic information from synthetic organisms to natural organisms. Importantly, this strategy is globally applicable to any gene or genetic system added to the synthetic organism. As the genetic code is near universally conserved we anticipate that the principles we have established may be applied to a broad range of other organisms.

### REFERENCES AND NOTES

1. F. H. Crick, L. Barnett, S. Brenner, R. J. Watts-Tobin, General nature of the genetic code for proteins. Nature 192, 1227-1232 (1961).
2. M. W. Nirenberg, J. H. Matthaei, The dependence of cell-free protein synthesis in E. coli upon naturally occurring or synthetic polyribonucleotides. Proc Natl Acad Sci USA 47, 1588-1602 (1961).
3. R. J. Hall, F. J. Whelan, J. O. McInerney, Y. Ou, M. R. Domingo-Sananes, Horizontal Gene Transfer as a Source of Conflict and Cooperation in Prokaryotes. Front Microbiol 11, 1569 (2020).
4. K. Vetsigian, C. Woese, N. Goldenfeld, Collective evolution and the genetic code. Proc Natl Acad Sci USA 103, 10696-10701 (2006).
5. D. de la Torre, J. W. Chin, Reprogramming the genetic code. Nat Rev Genet 22, 169-184 (2021).
6. E. V. Koonin, A. S. Novozhilov, Origin and evolution of the genetic code: the universal enigma. IUBMB Life 61, 99-111 (2009).
7. M. Kollmar, S. Muhlhausen, Nuclear codon reassignments in the genomics era and mechanisms behind their evolution. Bioessays 39, (2017).
8. J. Ling et al., Natural reassignment of CUU and CUA sense codons to alanine in Ashbya mitochondria. Nucleic Acids Res 42, 499-508 (2014).
9. A. L. Borges et al., Widespread stop-codon recoding in bacteriophages may regulate translation of lytic genes. Nat Microbiol 7, 918-927 (2022).
10. M. A. Santos, A. C. Gomes, M. C. Santos, L. C. Carreto, G. R. Moura, The genetic code of the fungal CTG clade. C R Biol 334, 607-611 (2011).
11. D. J. Taylor, M. J. Ballinger, S. M. Bowman, J. A. Bruenn, Virus-host co-evolution under a modified nuclear genetic code. PeerJ 1, e50 (2013).
12. Y. Shulgina, S. R. Eddy, A computational screen for alternative genetic codes in over 250,000 genomes. Elife 10, (2021).
13. D. G. Gibson et al., Complete chemical synthesis, assembly, and cloning of a Mycoplasma genitalium genome. Science 319, 1215-1220 (2008).
14. D. G. Gibson et al., One-step assembly in yeast of 25 overlapping DNA fragments to form a complete synthetic Mycoplasma genitalium genome. Proc Natl Acad Sci USA 105, 20404-20409 (2008).
15. J. Fredens et al., Total synthesis of Escherichia coli with a recoded genome. Nature 569, 514-+(2019).
16. F. J. Isaacs et al., Precise manipulation of chromosomes in vivo enables genome-wide codon replacement. Science 333, 348-353 (2011).
17. M. J. Lajoie et al., Genomically recoded organisms expand biological functions. Science 342, 357-360 (2013).
18. W. E. Robertson et al., Sense codon reassignment enables viral resistance and encoded polymer synthesis. Science 372, 1057-1062 (2021).
19. G. Pines, J. D. Winkler, A. Pines, R. T. Gill, Refactoring the Genetic Code for Increased Evolvability. mBio 8, (2017).
20. J. Calles, I. Justice, D. Brinkley, A. Garcia, D. Endy, Fail-safe genetic codes designed to intrinsically contain engineered organisms. Nucleic Acids Res 47, 10439-10451 (2019).
21. M. Schmidt, V. Kubyshkin, How To Quantify a Genetic Firewall?A Polarity-Based Metric for Genetic Code Engineering. Chembiochem 22, 1268-1284 (2021).
22. D. S. Marks, S. W. Michnick, Democratizing the mapping of gene mutations to protein biophysics. Nature 604, 47-48 (2022).
23. S. Teng, A. K. Srivastava, C. E. Schwartz, E. Alexov, L. Wang, Structural assessment of the effects of amino acid substitutions on protein stability and protein protein interaction. Int J Comput Biol Drug Des 3, 334-349 (2010).
24. V. Parthiban, M. M. Gromiha, D. Schomburg, CUPSAT: prediction of protein stability upon point mutations. Nucleic Acids Res 34, W239-242 (2006).
25. P. C. Ng, S. Henikoff, Predicting the effects of amino acid substitutions on protein function. Annu Rev Genomics Hum Genet 7, 61-80 (2006).
26. B. A. Renda, M. J. Hammerling, J. E. Barrick, Engineering reduced evolutionary potential for synthetic biology. Mol Biosyst 10, 1668-1678 (2014).
27. G. Moratorio et al., Attenuation of RNA viruses by redirecting their evolution in sequence space. Nat Microbiol 2, 17088 (2017).
28. J. R. Coleman et al., Virus attenuation by genome-scale changes in codon pair bias. Science 320, 1784-1787 (2008).
29. P. W. Barone et al., Viral contamination in biologic manufacture and implications for emerging therapies. Nat Biotechnol 38, 563-572 (2020).
30. P. Alamos et al., Functionality of tRNAs encoded in a mobile genetic element from an acidophilic bacterium. RNA Biol 15, 518-527 (2018).
31. T. Tuller et al., Association between translation efficiency and horizontal gene transfer within microbial communities. Nucleic Acids Res 39, 4743-4755 (2011).
32. J. W. Lee, C. T. Y. Chan, S. Slomovic, J. J. Collins, Next-generation biocontainment systems for engineered organisms. Nat Chem Biol 14, 530-537 (2018).

### Materials and Methods

### Strains

Throughout the text Syn61 WT refers to Syn61(ev2) (18) and Syn61Δ3 refers to Syn61Δ3(ev4) (18).

### Gene Recoding

For all genes and plasmids used in experiments in Syn61Δ3 derived cells it was necessary to compress the genetic code according to the recoding rules of Syn61 (TCG and TCA codons were replaced with AGC and AGT respectively, and the TAG stop codon was replaced with TAA). We recoded open reading frames as previously described for Syn61(15). The plasmids used in this study are provided in Data File S2 of Zurcher et al. “Refactored genetic codes enable bidirectional genetic isolation”, Science, provided herein as Table 1.

Construction of tRNA Plasmids for Decoding TCG and TCA Codons in Syn61Δ3

To incorporate amino acids in response to TCG and TCA codons we used pSC101-Kan and pSC101-Hyg plasmids (conferring resistance to kanamycin and hygromycin respectively) into which we cloned genes encoding the relevant tRNA or tRNAs. No exogenous aaRS was used, since all tRNAs used in this study are acylated by an endogenous E. coli aaRS. For tRNAs that incorporate amino acids other than serine in response to TCG and TCA codons, we designed genes in which the anticodon of the relevant isoacceptor tRNA was replaced with CGA and UGA respectively.

We constructed pSC101-based tRNA plasmids using HiFi Assembly of multiple fragments. Two different architectures of tRNA plasmids were used i) tRNAs were expressed under lpp promoter and the pSC101 backbone included a pheS-HygR double selection cassette expressed under an EM7 promoter, and ii) tRNAs were expressed using the native expression context of serT in the genome of E. coli and the pSC101 backbone included a kan^{R} expressed under a T3 promoter. In cases where two tRNAs were expressed from one plasmid they were expressed as an operon using the intergenic region between alaX and alaW tRNA genes in the E. coli genome. Backbone fragments were generated via PCR. tRNAs and tRNA operons were ordered as oligos (Merck) or gBlocks (IDT). All cloning was conducted in Syn61Δ3.

### Construction of Recoded Antibiotic Plasmids for Assessment of Genetic Code Orthogonality

To assess the functionality of antibiotic genes encoded according to different genetic codes we used pMB1-based plasmids containing a codon compressed antibiotic resistance gene into which we cloned genes encoding Hygromycin or Spectinomycin resistance (Data File S2). We constructed pMB1-based tRNA plasmids using HiFi Assembly of multiple fragments. Backbone fragments were generated via PCR. Recoded spectinomycin and hygromycin resistance genes were ordered as gBlocks (IDT).

### Construction of Recoded Mobile Genetic Elements

The intermediate, F (ΔTCG, TCA, TAG), was constructed from synthetic DNA (TWIST Bioscience) via yeast assembly (33). F (ΔTCG, TCA, TAG) was designed by recoding all annotated open reading frames in the RK2 conjugative plasmid, as previously described (15). The recoding of trfA to generate O-F1 (TCG-Ala, TCA-His) and O-F2 (TCG-His, TCA-Ala) was performed by lambda red recombination (34). Recoded versions of trfA were synthesized as gBlocks (IDT). All modifications were conducted in E. coli Dh10B. To enable the replication of F plasmids that were either lacking the trfA gene or encoding trfA in a genetic code not decodable by Dh10B, a pMB1 based helper plasmid expressing WT trfA in its endogenous context in the RK2 conjugative plasmid was used. This plasmid contained an amp^{R} gene expressed under the amp^{R} promoter and was assembled by HiFi Assembly from fragments generated by PCR.

sfGFP Expression Measurements

We expressed sfGFP-His6 genes bearing a single TCG or TCA codon at position 3 in Syn61Δ3 cells harboring a plasmid encoding a tRNA or tRNA operon. We electroporated 50 μL of Syn61Δ3 cells with pBAD_sfGFP reporter plasmid (100 ng) and recovered the cells in 1 mL of SOB for 90 min while shaking at 1050 rpm at 37° C. Subsequently, we inoculated the recovery culture (1 mL) into 5 mL of prewarmed 2×YT media containing 50 μg/mL apramycin and incubated cells overnight at 37° C. while shaking at 220 rpm before preparing electrocompetent cells. We electroporated pSC101-based tRNA plasmids (100 ng) into Syn61Δ3 cells with pBAD_sfGFP and recovered the cells in deep well 96-well plates for 90 min in 500 μL SOB. Subsequently, we inoculated a tenth of the recovery culture (50 μL) in 450 μL of prewarmed 2×YT media supplemented with 200 μg/mL hygromycin and 50 μg/mL apramycin. After recovering for 36 h, 37° C., 750 rpm, we setup expressions in 96-well microtiter plate format, inoculating overnight cultures 1:50 into 500 μL of prewarmed 2×YT containing hygromycin (200 ng/μL), apramycin (50 ng/μL), and L-arabinose (0.2%). The expressions were incubated for 16 h at 37° C. while shaking at 750 rpm. Plates were centrifuged at 3200 g for 10 min. We resuspended cell pellets in 150 μL of PBS, 100 μL of which we transferred into a Costar clear 96-well flat-bottom plate. In this plate we recorded OD₆₀₀ and GFP fluorescence (μₑₓ: 485 nm; λₑₘ: 520 nm) measurements on a PHERAstar FS plate reader (BMG LABTECH) (gain setting of 0, focal adjustment of 00 mm).

To determine the protein yield sfGFP (WT) was expressed in Syn61Δ3 (16 h at 37° C. in 5 mL of 2×TY+0.2% arabinose) and purified as described below (three elutions 100 μL each). Protein concentration post-purification was determined by nanodrop (elution 1: 0.77 mg/mL; elution 2: 0.09 mg/mL; elution 3: −0.0 mg/mL). The measured amount adds up to 0.086 mg of protein extracted from 5 mL of culture, which corresponds to a protein yield of −17 mg/L of culture.

Purification of sfGFP-His6x and Ubiquitin-His6x Proteins

Syn61Δ3 cells harbouring a pSC101-based tRNA plasmid and a pBAD_sfGFP (or Ubiquitin) plasmid were grown for 16 h in 5 mL (20 mL for Ubiquitin) 2×YT media containing 200 μg/mL hygromycin, 50 μg/mL apramycin, and 0.2% L-arabinose at 37° C. while shaking at 220 rpm. Following the expression, cells were centrifuged, resuspended in 1 mL Lysis buffer (1× Bugbuster Protein Extraction Reagent (Novagen), 1×PBS, 50 μg/mL DNase 1, 20 mM imidazole, and 100 μg/mL lysozyme), and incubated at 4° C. for 1 h. The resulting lysates were centrifuged (16000×g) at 4° C. for 30 min. The supernatant was then transferred to 1.5 mL microcentrifuge tubes containing 50 μL of Ni²⁺-NTA slurry (Qiagen) and incubated for 1 h at 4° C. while tumbling. Ni2+-NTA beads were collected by gravity filtration on a fritted column and washed three times in 500 μL wash buffer (1×PBS, 40 mM imidazole). Lastly, proteins were eluted in 100 μL of elution buffer (1×PBS, 300 mM imidazole, pH 8) and collected in a fresh microcentrifuge tube via centrifugation (100×g, 4° C., 1 min).

### Intact Protein Mass Spectrometry

ESI-MS analysis of proteins (Ubiquitin FIG. 13 and sfGFP FIG. 8) was performed using a Waters Xevo G2 mass spectrometer coupled to a modified nanoAcquity LC system. The purified samples (as described above) were separated on a BEH C4 UPLC column (1.7 μm; 1.0×100 mm; Waters) over 20 min with a flowrate of 50 UL/min and a water/acetonitrile gradient from 2% vol/vol to 80% vol/vol. Subsequently, eluted samples were interfaced via Zspray electrospray ionization source with the mass spectrometer (Waters). Data were acquired in positive ion mode with a range from 300-2000 m/z and an applied cone voltage of 30 V. Spectra were deconvoluted using the MaxEnt1 function within MassLynx software (Waters). To calculate the expected molecular weights, the expected mass of wild-type proteins was determined using GPMAW (Lighthouse Data) and then manually edited to accommodate encoded amino acid changes.

ESI-MS analysis of proteins (sfGFP FIG. 2B, FIG. 14, FIG. 15) was performed using a Waters Vion IMS Qtof mass spectrometer coupled to a modified nanoAcquity LC system. The purified samples (as described above) were separated on an Acuity UPLC protein BEH C4 column (1.7 um; 2.1×50 mm; Waters) over 7 min with a flowrate of 200 UL/min and a water/acetonitrile gradient from 5% vol/vol to 100% vol/vol. Subsequently, eluted samples were interfaced via Zspray electrospray ionization source with the mass spectrometer (Waters). Data were acquired in positive ion mode with a range from 100-2000 m/z. Spectra were deconvoluted using the MaxEnt1 function within Unifi software (Waters). To calculate the expected molecular weights, the expected mass of wild-type proteins was determined using GPMAW (Lighthouse Data) and then manually edited to accommodate encoded amino acid changes.

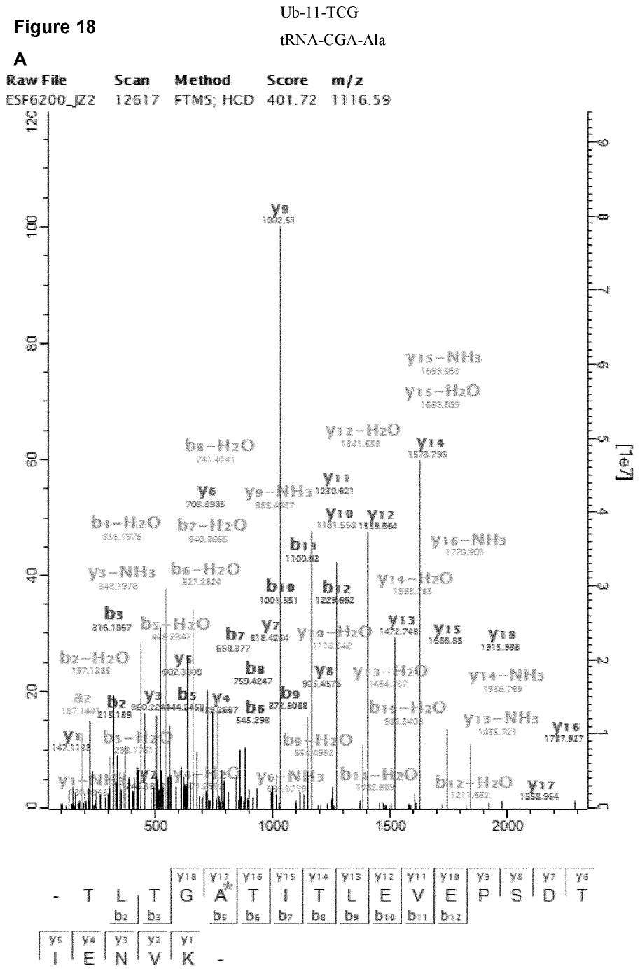

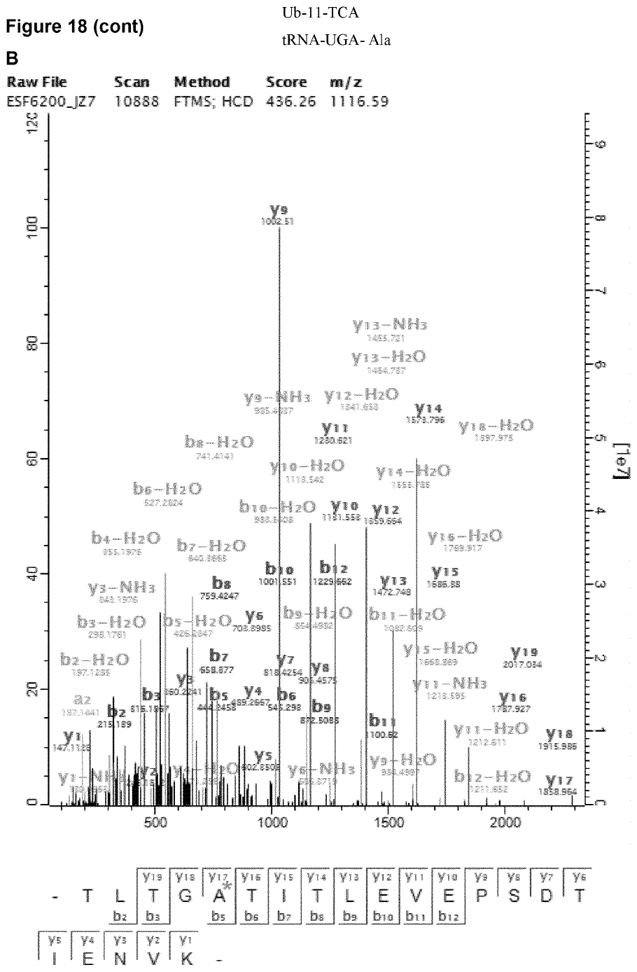

Mass spectra of ubiquitin in the screen of anticodon modified tRNAs (FIG. 18) were acquired on an Agilent 1200 LC-MS system equipped with a 6130 Quadrupole spectrometer. Proteins were eluted from a Phenomenex Jupiter C4 column (150×2 mm, 5 μm). Reverse-phase HPLC was performed using Buffer A (0.2% formic acid in water) and buffer B (0.2% formic acid in acetonitrile (MeCN)). Mass spectra were acquired in positive mode and analyzed with MS Chemstation software (Agilent Technologies). The deconvolution program provided in the software was used to obtain the entire mass spectra.

### Calculating Signal to Noise and Fidelity Measurements in ESI-MS Spectra

For ESI-spectra of sfGFP, we calculated the average signal intensity, and standard deviation of intensities, between 20,000 Da and 27,000 Da. We defined the noise as the average signal intensity plus twice the standard deviation for this mass window. The limit of fidelity measurement was calculated as: (1−(N/S))×100. (Note: for the spectra in FIG. S3 the baseline signal was determined between 20,000 Da and 26,500 Da because there is a peak from a degradation product around 27,000 Da). This calculation defines the maximum fidelity that can be obtained from the spectra, we note that the true biological fidelity may be higher.

To determine the specificity for decoding TCG codons in the presence of both tRNA_{CGA}^{XXX} and tRNA_{UGA}^{YYY}, where XXX and YYY are distinct amino acids, we divided the intensity of the signal at the peak resulting from incorporation of XXX at TCG by the intensity of the signal at the expected mass for incorporating YYY at TCG. The intensity at the expected mass for incorporating YYY at TCG was determined as the maximum signal in a 2 Da window around the theoretical calculated mass.

To determine the specificity for decoding TCA codons in the presence of both tRNA_{CGA}^{XXX} and tRNA_{UGA}^{YYY}, where XXX and YYY are distinct amino acids, we divided the intensity of the signal at the peak resulting from incorporation of YYY at TCA by the intensity of the signal at the expected mass for incorporating XXX at TCA. The intensity at the expected mass for incorporating XXX at TCA was determined as the maximum signal in a 2 Da window around the theoretical calculated mass.

Western Blotting of Cell Lysates from Experiments of Ubiquitin Expression

Syn61Δ3 cells harboring a pSC101-based tRNA plasmid and a pBAD_Ubiquitin plasmid were grown for 16 h in 20 mL of 2×TY media containing 200 μg/mL hygromycin, 50 μg/mL apramycin, and 0.2% L-arabinose at 37° C. while shaking at 220 rpm. Cultures were normalized to OD600=1.0. 500 μL of normalized culture were lysed with sample buffer (Nupage Buffer, 10% beta mercaptoethanol, PMSF) and vortexed intensively to shear DNA. Samples were separated by SDS-PAGE (NuPAGE 4-12% in MES buffer and transferred to polyvinylidene difluoride (PVDF) membrane by iBlot 2 dry blotting system (Thermo Fisher Scientific). Membrane was blocked by Odyssey blocking buffer in PBS (catalogue (cat.) no. 927-40000, Li-Cor) at room temperature for 30 min. Membrane was incubated with anti-His-tag primary antibody (Abcam, cat. no. ab18184) in primary antibody solution (dilution 1:1000 in Odyssey T20 (PBS) antibody diluent (927-75001, Li-Cor)) at 4° C. overnight. All incubations were carried out on a platform shaker. The membrane was washed three time with PBST (PBS supplemented with 0.1% Tween-20 (v/v)), and incubated with the secondary antibody Goat anti-Mouse IRDye 680RD 925-68070 (1:15,000 (v/v) in PBS blocking buffer supplemented with 0.2% Tween-20 (v/v), and 0.01% SDS) at room temperature for 1 h. After washing 3 times with PBST and once with PBS, the immunoreactive proteins were visualized on a Typhoon Trio phosphorimager (GE Life Sciences). Samples analysed by Western blotting were also separated by SDS-PAGE and the gel was stained with InstantBlue (Expedeon) for 30 min followed by a rinse with water.

### MS/MS of Ubiquitin Variants

Solutions samples were reduced with dithiothreitol at 37° C. and alkylated with chloroacetamide in the dark at room temperature. Samples were digested with LysC (Promega) at 37° C. for 4 h followed by trypsin (Promega) digestion over night at 37° C. The peptide mixtures were acidified and desalted using home-made C18 (3M Empore) stage tips that contained 3 μl of Poros Oligo R3 (Thermo Fisher Scientific) resin. Bound peptides were eluted from stage tip with 30-80% acetonitrile (MeCN) and partially dried down in a Speed Vac (Savant).

Peptides were separated on an Ultimate 3000 RSLC nano System (Thermo Scientific), fitted with a 75 μm×25 cm, nanoEase C18 T3 column (Waters), using mobile phases buffer A (2% MeCN, 0.1% formic acid) and buffer B (80% MeCN, 0.1% formic acid). Eluted peptides were introduced directly via a nanospray ion source into a Q Exactive Plus hybrid quardrupole-Orbitrap mass spectrometer (Thermo Fisher Scientific). The mass spectrometer was operated in data dependent mode. MS1 spectra were acquired from 380-1600 m/z, at a resolution of 70000, followed by MS2 acquisitions of the 15 most intense ions with a resolution of 17500 and NCE of 27%. MS target values of 1e6 and MS2 target values of 1e5 were used. Dynamic exclusion was set for 30 s.

The acquired raw data files were searched against E. coli UniProt Fasta database (downloaded September 2022), with an additional 20 ubiquitin sequences (each sequence had a different canonical amino acid at position 11), using MaxQuant with the integrated Andromeda search engine (v.1.6.17.0). Carbamidomethylation of cysteine was set as fixed modification while oxidation of methionine as variable modifications. Enzyme specificity was set to trypsin/p and a maximum two missed cleavages were allowed.

### Preparation of Electrocompetent Syn61Δ3 Cells and Electroporation

250 mL of prewarmed 2×YT medium were inoculated with 5 mL of Syn61Δ3 overnight culture and grown at 37° C. while shaking (220 rpm) to an OD₆₀₀ of ˜0.5. The cells were chilled on ice for 10 min and harvested by centrifugation (4000 rpm, 10 min, 4° C.). After washing cell pellets three times in 50 mL of ice-cold 20% glycerol they were resuspended in a final volume of 500 μL of ice-cold 20% glycerol and frozen in liquid nitrogen in aliquots of 100 μL. For electroporation frozen cells were thawed on ice and 50 μL of cells were mixed with 100 ng of plasmid DNA. The mixture was placed in an electroporation cuvette (2 mm gap; SLS scientific) and electroporated using an Eppendorf e-porator (2500 V). Cells were immediately resuspended in 1 mL of prewarmed SOB outgrowth media, transferred into a 2 mL microcentrifuge tube, and incubated at 37° C. for 90 min while shaking (1050 rpm). Subsequently, we inoculated the recovery culture (1 mL) into 5 mL of prewarmed 2×YT media containing appropriate antibiotics and incubated cells overnight at 37° C. while shaking at 220 rpm.

### Conjugation Assay

Donor and recipient cells for conjugation assays were grown overnight in 5 mL 2×YT in the presence of appropriate antibiotics (50 μg/mL kanamycin for recipients; 20 μg/mL chloramphenicol for donors). The OD₆₀₀ of cultures was determined and cultures were normalized to OD₆₀₀=2.0. 400 μL of culture were then transferred to a 2 mL microcentrifuge tube and washed twice with 2×YT. After washing pellets were resuspended in a final volume on 200 μL. For conjugations 100 μL of donor and 100 μL of recipient were mixed and spotted in 5 μL drops on a TYE plate. The plate was then incubated at 37° C. for 2 h. Subsequently, cells were washed from the plate using 2 mL 2×YT and transferred to a fresh 2 mL microcentrifuge tube. Cells were pelleted by centrifugation (1 min, 3000×g), resuspended in 1 mL H₂O, and diluted in series (1:10). Dilutions ranging 10⁰-10⁻⁷ were spotted (3 μL drops) on a 2×YT Agar plate containing 50 μg/mL kanamycin and 20 μg/mL chloramphenicol. Plates were incubated 24 to 36 h at 37° C. and colonies were counted manually to determine the number of successful transconjugants. For experiments with code-locking the appropriate antibiotic (200 μg/mL hygromycin or 75 μg/mL spectinomycin) was added to the 2×TY agar plates.

### Doubling Time Measurements

Cells were inoculated in a Costar clear 96-well flat-bottom plate from dense overnight culture (1:100 ratio) in 200 μL 2×TY containing 200 μg/mL hygromycin. Cells were grown at 37° C. shaking (880 rpm) in a TECAN infinite M200 Pro. Every 5 min, over a 24 h period, we took an OD₆₀₀ measurement to determine cell density. A sliding window of 10 time points was used to determine the area with the steepest slope of the growth curve. Doubling times were determined from this area.

Phage Enrichments from Environmental Samples

Water samples were collected from different locations alongside the River Cam (Cambridge, United Kingdom). After filtration through a 0.22 μm filter, 4 mL of a water sample was mixed with 4 mL of 2×LB and 200 μl of an overnight culture of E. coli, followed by a 48 h incubation at 37° C. in a rotary wheel. Then, cultures were centrifuged at 4500×g for 15 mins and the filtered supernatant was kept as a phage enrichment.

### Note: Locations

A: Cambridge Water Treatment plant outflow (52°13′55.3″N 0°10′15.3″E); B: Grassy Corner (52°13′21.3″N 0°10′00.0″E); C: Coffee Temple (52°13′07.7″N 0°09′01.5″E); D: Green Dragon Bridge (52°13′02.9″N 0°08′44.8″E); D: Jesus Green's Lock (52°12′45.8″N 0°07′15.4″E); E: Scudamore's at Granta Place (52°12′04.6″N 0°06′56.8″E).

### Plaque Purification and Phage Lysate Preparation

To purify phages plaques, phage enrichments were serially diluted (10-fold) in LB and 10 μl of each dilution was added to a bijoux bottle with 200 μL of an overnight culture of the bacterial host to assess. Then 4 mL of molten top agar (0.35% agarose) was added, mixed, and poured as an overlay on LB agar plates containing the appropriate antibiotics. The resulting plates were incubated at 37° C. overnight. We picked Individual phage plaques using a sterile toothpick and resuspended them in 100 μl of LB. The mixture was spun down and the supernatant was diluted and used for further purification rounds as described above. This process was repeated three times to ensure phage purity.

Phage lysates were collected from bacterial lawns exhibiting near-confluent lysis after infection by pure phage isolates. Top agar was scraped into a glass universal bottle containing 3 ml of LB and homogenized using a sterile pipette. The suspension was then centrifuged (4500×g, 4° C., 20 min). The obtained supernatants were filtered through a 0.22 μm filter and stored in bijoux bottles at 4° C. Phage titer was estimated by counting the number of plaques obtained from phage lysate dilutions, plated out, as described above.

### Phage DNA Extraction

Genomic DNA of phages was obtained from 450 μL of high-titer phage lysates (˜10¹⁰ PFU/mL) using a standard phenol/chloroform method as described by Chen et al. (2017) (43).

### Efficiency of Plaquing Assays

Phage lysates were serially diluted (10-fold) in LB. Dilutions were spotted (7.5 μL per spot) on freshly poured and dried top lawns (200 μL of overnight culture mixed with 4 mL of top agar poured as an overlay on LB agar plates containing 200 μg/mL hygromycin and 75 μg/mL spectinomycin) and incubated overnight at 37° C. Images of spots were taken on an iPhone 8 and converted to gray scale in Adobe illustrator. For concentrations where single plaques were expected full top lawns were poured to get a better assessment of plaque forming units at the given concentration (200 μL of overnight culture mixed with 10 μL of phage lysate at the concentration of interest and 4 mL of top agar poured as an overlay on LB agar plates containing 200 μg/mL hygromycin and 75 μg/mL spectinomycin). All plaque counts displayed in bar graphs stem from full top lawns. For titer lysates (>10⁶ PFU/mL) full top lawns were poured as described above to avoid lysis from without. The maximum titers used for infections with phage 06 and phage 12 were ˜7.5×10⁹ PFU/mL and ˜1.1×10¹⁰ PFU/mL respectively. The strains used in this experiment contain different versions of spectinomycin resistance genes. Syn61 WT contains a SpecR WT, Syn61Δ3 contains recSpecR, Syn61Δ3 (tRNA_{CGA}^{Ala}, tRNA_{UGA}^{His}) contains O-SpecR (TCG-Ala, TCA-His), Syn61Δ3 (tRNA_{CGA}^{Ala}, tRNA_{UGA}^{Leu}) contains O-SpecR (TCG-Ala, TCA-Leu), Syn61Δ3 (tRNA_{CGA}^{Leu}, tRNA_{UGA}^{Leu}) contains O-SpecR (TCG-Leu, TCA-Leu), Syn61Δ3 (tRNA_{CGA}^{Pro}, tRNA_{UGA}^{Leu}) contains O-SpecR (TCG-Pro, TCA-Leu).

### Electron Microscopy

Phage samples were prepared by adsorbing 10 μL of a high titer phage lysate (>10⁹ PFU/mL) onto a charged copper grid and stained with 2% (w/v) uranyl acetate. Transmission Electron Micrograph (TEM) images were taken at the Cambridge Advanced Imaging Centre (CAIC), University of Cambridge using a FEI Tecnai G2 series transmission electron microscope (Accelerating voltage: 200.0 kV; Direct magnification: 50,000×).

### Phage Genome Sequencing and De Novo Assembly

Purified phage DNA was prepared for NGS using the Nextera XT DNA library preparation kit. Libraries were paired-end sequenced on a MiSeq (Illumina, reagent kit v3 (150 cycles)). De novo assembly of phage genomes was performed with Unicycler in short-read mode and with default options. Sequence coverage throughout the phage genome is represented as median sequencing coverage in windows of 250 bp.

tRNA Screen

An overview of the sequences used in the tRNA screen are provided in SEQ ID NOs: 7-68. These sequences represent, in order, ArgX (anticodon modified to CGA), ArgX (anticodon modified to TGA), ArgW (anticodon modified to CGA), ArgW (anticodon modified to TGA), ileT (anticodon modified to CGA), ileT (anticodon modified to TGA), PheU (anticodon modified to CGA), PheU (anticodon modified to TGA), AspT (anticodon modified to CGA), AspT (anticodon modified to TGA), AsnT (anticodon modified to CGA), AsnT (anticodon modified to TGA), GltU (anticodon modified to CGA), GltU (anticodon modified to TGA), ValV (anticodon modified to CGA), ValV (anticodon modified to TGA), ThrT (anticodon modified to CGA), ThrT (anticodon modified to TGA), ThrU (anticodon modified to CGA), ThrU (anticodon modified to TGA), GlyU (anticodon modified to CGA), GlyU (anticodon modified to TGA), GlyT (anticodon modified to CGA), GlyT (anticodon modified to TGA), GlnU (anticodon modified to CGA), GlnU (anticodon modified to TGA), GlnV (anticodon modified to CGA), GlnV (anticodon modified to TGA), MetV (anticodon modified to CGA), MetV (anticodon modified to TGA), MetY (anticodon modified to CGA), MetY (anticodon modified to TGA), ThrV (anticodon modified to CGA), ThrV (anticodon modified to TGA), valW (anticodon modified to CGA), valW (anticodon modified to TGA), ArgQ (anticodon modified to CGA), ArgQ (anticodon modified to TGA), ArgV (anticodon modified to CGA), ArgV (anticodon modified to TGA), CysT (anticodon modified to CGA), CysT (anticodon modified to TGA), HisR (anticodon modified to CGA), HisR (anticodon modified to TGA), ileX (anticodon modified to CGA), ileX (anticodon modified to TGA), LysQ (anticodon modified to CGA), LysQ (anticodon modified to TGA), ProK (anticodon modified to CGA), ProK (anticodon modified to TGA), ProL (anticodon modified to CGA), ProL (anticodon modified to TGA), ProM (anticodon modified to CGA), ProM (anticodon modified to TGA), TrpT (anticodon modified to CGA), TrpT (anticodon modified to TGA), TyrV (anticodon modified to CGA), TyrV (anticodon modified to TGA), AlaT (anticodon modified to CGA), AlaT (anticodon modified to TGA), LeuQ (anticodon modified to CGA), LeuQ (anticodon modified to TGA).

TABLE 1

| Plasmid | Description | Genbank # | Reference |
|---|---|---|---|
| Helper | Contains lambda-red recombination components and | MN927219 | Wang et al. |
| | Cas9 under arabinose inducible promoter as well as | | |
| | tracrRNA | | |
| pBAD_sfGFP3TCG | Recoded sfGFP reporter (as in Genbank accession | none | Robertson e |
| | MW879733, without the aaRS/tRNA pair) with TCG | | |
| | inserted immediately after codon 2 of sfGFP | | |
| pBAD_sfGFP3TCA | Recoded sfGFP reporter (as in Genbank accession | none | Robertson e |
| | MW879733, without the aaRS/tRNA pair) with TCA | | |
| | inserted immediately after codon 2 of sfGFP | | |
| | | none | This study |
| pSC101_HygR_alaT_CGA | Recoded pheS-HygRdoubleselectioncassette alaT tRNA | none | This study |
| | with CGA chimeric anticodon under lpp promoter | | |
| pSC101_HygR_alaT_TGA | Recoded pheS-HygRdoubleselectioncassette alaT tRNA | none | This study |
| | with TGA chimeric anticodon under lpp promoter | | |
| pSC101_HygR_hisR_CGA | Recoded pheS-HygRdoubleselectioncassette hisR tRNA | none | This study |
| | with CGA chimeric anticodon under lpp promoter | | |
| pSC101_HygR_hisR_TGA | Recoded pheS-HygRdoubleselectioncassette hisR tRNA | none | This study |
| | with TGA chimeric anticodon under lpp promoter | | |
| pSC101_HygR_leuQ_CGA | Recoded pheS-HygRdoubleselectioncassette leuQ tRNA | none | This study |
| | with CGA chimeric anticodon under lpp promoter | | |
| pSC101_HygR_leuQ_TGA | Recoded pheS-HygRdoubleselectioncassette leuQ tRNA | none | This study |
| | with TGA chimeric anticodon under lpp promoter | | |
| pSC101_HygR_proM_CGA | Recoded pheS-HygRdoubleselectioncassette proM tRNA | none | This study |
| | with CGA chimeric anticodon under lpp promoter | | |
| pSC101_HygR_proM_TGA | Recoded pheS-HygRdoubleselectioncassette proM tRNA | none | This study |
| | with TGA chimeric anticodon under lpp promoter | | |
| pSC101_HygR_serT | Recoded pheS-HygRdoubleselectioncassette serT tRNA | none | This study |
| | under lpp promoter | | |
| pSC101_HygR_serU | Recoded pheS-HygRdoubleselectioncassette serU tRNA | none | This study |
| | under lpp promoter | | |
| pSC101_HygR_ctrl | Recoded pheS-HygRdoubleselectioncassette no tRNA | none | This study |
| pSC101_HygR_alaT_ | Recoded pheS-HygRdoubleselectioncassette alaT tRNA | none | This study |
| CGA_alaT_TGA | with CGA chimeric anticodon and alaT tRNA with TGA | | |
| | anticodon under lpp promoter | | |
| pSC101_HygR_alaT_ | Recoded pheS-HygRdoubleselectioncassette alaT tRNA | none | This study |
| CGA_hisR_TGA | with CGA chimeric anticodon and hisR tRNA with TGA | | |
| | anticodon under lpp promoter | | |
| pSC101_HygR_alaT_ | Recoded pheS-HygRdoubleselectioncassette alaT tRNA | none | This study |
| CGA_leuQ_TGA | with CGA chimeric anticodon and leuQ tRNA with TGA | | |
| | anticodon under lpp promoter | | |
| pSC101_HygR_alaT_ | Recoded pheS-HygRdoubleselectioncassette alaT tRNA | none | This study |
| CGA_proM_TGA | with CGA chimeric anticodon and proM tRNA with TGA | | |
| | anticodon under lpp promoter | | |
| pSC101_HygR_hisR_ | Recoded pheS-HygRdoubleselectioncassette hisR tRNA | none | This study |
| CGA_alaT_TGA | with CGA chimeric anticodon and alaT tRNA with TGA | | |
| | anticodon under lpp promoter | | |
| pSC101_HygR_hisR_ | Recoded pheS-HygRdoubleselectioncassette hisR tRNA | none | This study |
| CGA_hisR_TGA | with CGA chimeric anticodon and hisR tRNA with TGA | | |
| | anticodon under lpp promoter | | |
| pSC101_HygR_hisR_ | Recoded pheS-HygRdoubleselectioncassette hisR tRNA | none | This study |
| CGA_leuQ_TGA | with CGA chimeric anticodon and leuQ tRNA with TGA | | |
| | anticodon under lpp promoter | | |
| pSC101_HygR_hisR_ | Recoded pheS-HygRdoubleselectioncassette hisR tRNA | none | This study |
| CGA_proM_TGA | with CGA chimeric anticodon and proM tRNA with TGA | | |
| | anticodon under lpp promoter | | |
| pSC101_HygR_leuQ_ | Recoded pheS-HygRdoubleselectioncassette leuQ tRNA | none | This study |
| CGA_alaT_TGA | with CGA chimeric anticodon and alaT tRNA with TGA | | |
| | anticodon under lpp promoter | | |
| pSC101_HygR_leuQ_ | Recoded pheS-HygRdoubleselectioncassette leuQ tRNA | none | This study |
| CGA_hisR_TGA | with CGA chimeric anticodon and hisR tRNA with TGA | | |
| | anticodon under lpp promoter | | |
| pSC101_HygR_leuQ_ | Recoded pheS-HygRdoubleselectioncassette leuQ tRNA | none | This study |
| CGA_leuQ_TGA | with CGA chimeric anticodon and leuQ tRNA with TGA | | |
| | anticodon under lpp promoter | | |
| pSC101_HygR_leuQ_ | Recoded pheS-HygRdoubleselectioncassette leuQ tRNA | none | This study |
| CGA_proM_TGA | with CGA chimeric anticodon and proM tRNA with TGA | | |
| | anticodon under lpp promoter | | |
| pSC101_HygR_proM_ | Recoded pheS-HygRdoubleselectioncassette proM tRNA | none | This study |
| CGA_alaT_TGA | with CGA chimeric anticodon and alaT tRNA with TGA | | |
| | anticodon under lpp promoter | | |
| pSC101_HygR_proM_ | Recoded pheS-HygRdoubleselectioncassette proM tRNA | none | This study |
| CGA_hisR_TGA | with CGA chimeric anticodon and hisR tRNA with TGA | | |
| | anticodon under lpp promoter | | |
| pSC101_HygR_proM_ | Recoded pheS-HygRdoubleselectioncassette proM tRNA | none | This study |
| CGA_leuQ_TGA | with CGA chimeric anticodon and leuQ tRNA with TGA | | |
| | anticodon under lpp promoter | | |
| pSC101_HygR_proM_ | Recoded pheS-HygRdoubleselectioncassette proM tRNA | none | This study |
| CGA_proM_TGA | with CGA chimeric anticodon and proM tRNA with TGA | | |
| | anticodon under lpp promoter | | |
| pSC101_KanR_alaT_CGA | Recoded KanR alaT tRNA with CGA chimeric anticodon | none | This study |
| | in endogenous genomic context of serT | | |
| pSC101_KanR_alaT_TGA | Recoded KanR alaT tRNA with TGA chimeric anticodon | none | This study |
| | in endogenous genomic context of serT | | |
| pSC101_KanR_hisR_CGA | Recoded KanR hisR tRNA with CGA chimeric anticodon | none | This study |
| | in endogenous genomic context of serT | | |
| pSC101_KanR_hisR_TGA | Recoded KanR hisR tRNA with TGA chimeric anticodon | none | This study |
| | in endogenous genomic context of serT | | |
| pSC101_KanR_leuQ_CGA | Recoded KanR leuQ tRNA with CGA chimeric anticodon | none | This study |
| | in endogenous genomic context of serT | | |
| pSC101_KanR_leuQ_TGA | Recoded KanR leuQ tRNA with TGA chimeric anticodon | none | This study |
| | in endogenous genomic context of serT | | |
| pSC101_KanR_proM_CGA | Recoded KanR proM tRNA with CGA chimeric anticodon | none | This study |
| | in endogenous genomic context of serT | | |
| pSC101_KanR_proM_TGA | Recoded KanR proM tRNA with TGA chimeric anticodon | none | This study |
| | in endogenous genomic context of serT | | |
| pSC101_KanR_serT | Recoded KanR serT tRNA in endogenous genomic | none | This study |
| | context of serT | | |
| pSC101_KanR_serU | Recoded KanR serU tRNA in endogenous genomic | none | This study |
| | context of serT | | |
| pSC101_KanR_ctrl | Recoded KanR no tRNA | none | This study |
| pSC101_KanR_alaT_ | Recoded KanR alaT tRNA with CGA chimeric anticodon | none | This study |
| CGA_alaT_TGA | and alaT tRNA with TGA anticodon in endogenous | | |
| | genomic context of serT | | |
| pSC101_KanR_alaT_ | Recoded KanR alaT tRNA with CGA chimeric anticodon | none | This study |
| CGA_hisR_TGA | and hisR tRNA with TGA anticodon in endogenous | | |
| | genomic context of serT | | |
| pSC101_KanR_alaT_ | Recoded KanR alaT tRNA with CGA chimeric anticodon | none | This study |
| CGA_leuQ_TGA | and leuQ tRNA with TGA anticodon in endogenous | | |
| | genomic context of serT | | |
| pSC101_KanR_alaT_ | Recoded KanR alaT tRNA with CGA chimeric anticodon | none | This study |
| CGA_proM_TGA | and proM tRNA with TGA anticodon in endogenous | | |
| | genomic context of serT | | |
| pSC101_KanR_hisR_ | Recoded KanR hisR tRNA with CGA chimeric anticodon | none | This study |
| CGA_alaT_TGA | and alaT tRNA with TGA anticodon in endogenous | | |
| | genomic context of serT | | |
| pSC101_KanR_hisR_ | Recoded KanR hisR tRNA with CGA chimeric anticodon | none | This study |
| CGA_hisR_TGA | and hisR tRNA with TGA anticodon in endogenous | | |
| | genomic context of serT | | |
| pSC101_KanR_hisR_ | Recoded KanR hisR tRNA with CGA chimeric anticodon | none | This study |
| CGA_leuQ_TGA | and leuQ tRNA with TGA anticodon in endogenous | | |
| | genomic context of serT | | |
| pSC101_KanR_hisR_ | Recoded KanR hisR tRNA with CGA chimeric anticodon | none | This study |
| CGA_proM_TGA | and proM tRNA with TGA anticodon in endogenous | | |
| | genomic context of serT | | |
| pSC101_KanR_leuQ_ | Recoded KanR leuQ tRNA with CGA chimeric anticodon | none | This study |
| CGA_alaT_TGA | and alaT tRNA with TGA anticodon in endogenous | | |
| | genomic context of serT | | |
| pSC101_KanR_leuQ_ | Recoded KanR leuQ tRNA with CGA chimeric anticodon | none | This study |
| CGA_hisR_TGA | and hisR tRNA with TGA anticodon in endogenous | | |
| | genomic context of serT | | |
| pSC101_KanR_leuQ_ | Recoded KanR leuQ tRNA with CGA chimeric anticodon | none | This study |
| CGA_leuQ_TGA | and leuQ tRNA with TGA anticodon in endogenous | | |
| | genomic context of serT | | |
| pSC101_KanR_leuQ_ | Recoded KanR leuQ tRNA with CGA chimeric anticodon | none | This study |
| CGA_proM_TGA | and proM tRNA with TGA anticodon in endogenous | | |
| | genomic context of serT | | |
| pSC101_KanR_proM_ | Recoded KanR proM tRNA with CGA chimeric anticodon | none | This study |
| CGA_alaT_TGA | and alaT tRNA with TGA anticodon in endogenous | | |
| | genomic context of serT | | |
| pSC101_KanR_proM_ | Recoded KanR proM tRNA with CGA chimeric anticodon | none | This study |
| CGA_hisR_TGA | and hisR tRNA with TGA anticodon in endogenous | | |
| | genomic context of serT | | |
| pSC101_KanR_proM_ | Recoded KanR proM tRNA with CGA chimeric anticodon | none | This study |
| CGA_leuQ_TGA | and leuQ tRNA with TGA anticodon in endogenous | | |
| | genomic context of serT | | |
| pSC101_KanR_proM_ | Recoded KanR proM tRNA with CGA chimeric anticodon | none | This study |
| CGA_proM_TGA | and proM tRNA with TGA anticodon in endogenous | | |
| | genomic context of serT | | |
| pMB1_recAmpR_specR_ | Codon compressed amicilin resistance and | none | This study |
| WT | spectinomycin resistance encoded according to WT | | |
| | genetic code | | |
| pMB1_recAmpR_specR_ | Codon compressed amicilin resistance and codon | none | This study |
| rec | compressed spectinomycin resistance | | |
| pMB1_recAmpR_specR_ | Codon compressed amicilin resistance and | none | This study |
| reassigned | spectinomycin resistance encoded according to | | |
| | orthogonal genetic code (Ala-TCG, His-TCA) | | |
| pMB1_recAmpR_hygR_ | Codon compressed amicilin resistance and hygromycin | none | This study |
| WT | resistance encoded according to WT genetic code | | |
| pMB1_recAmpR_hygR_ | Codon compressed amicilin resistance and codon | none | This study |
| rec | compressed hygromycin resistance | | |
| pMB1_recAmpR_hygR_ | Codon compressed amicilin resistance and hygromycin | none | This study |
| reassigned | resistance encoded according to orthogonal genetic | | |
| | code (Ala-TCG, His-TCA) | | |
| F WT | RK2 conjugation plasmid containing recoded | none | This study |
| | chloramphenicol resistance gene (based on | | |
| | BN000925.1) | | |
| F WT + serT | RK2 conjugation plasmid containing recoded | none | This study |
| | chloramphenicol resistance gene and serT tRNA gene | | |
| | (based on BN000925.1) | | |
| O-F1 | RK2 derived plasmid constructed from synthetic DNA. | none | This study |
| | Recoded according to Syn61 recoding scheme. trfA | | |
| | gene additionally recoded (Ala-TCG, His-TC | | |
| O-F2 | RK2 derived plasmid constructed from synthetic DNA. | none | This study |
| | Recoded according to Syn61 recoding scheme. trfA | | |
| | gene additionally recoded (His-TCG, Ala-TC | | |

### REFERENCES TO MATERIALS AND METHODS

1. F. H. Crick, L. Barnett, S. Brenner, R. J. Watts-Tobin, General nature of the genetic code for proteins. Nature 192, 1227-1232 (1961).
2. M. W. Nirenberg, J. H. Matthaei, The dependence of cell-free protein synthesis in E. coli upon naturally occurring or synthetic polyribonucleotides. Proc Natl Acad Sci USA 47, 1588-1602 (1961).
3. R. J. Hall, F. J. Whelan, J. O. McInerney, Y. Ou, M. R. Domingo-Sananes, Horizontal Gene Transfer as a Source of Conflict and Cooperation in Prokaryotes. Front Microbiol 11, 1569 (2020).
4. K. Vetsigian, C. Woese, N. Goldenfeld, Collective evolution and the genetic code. Proc Natl Acad Sci USA 103, 10696-10701 (2006).
5. D. de la Torre, J. W. Chin, Reprogramming the genetic code. Nat Rev Genet 22, 169-184 (2021).
6. E. V. Koonin, A. S. Novozhilov, Origin and evolution of the genetic code: the universal enigma. IUBMB Life 61, 99-111 (2009).
7. M. Kollmar, S. Muhlhausen, Nuclear codon reassignments in the genomics era and mechanisms behind their evolution. Bioessays 39, (2017).
8. J. Ling et al., Natural reassignment of CUU and CUA sense codons to alanine in Ashbya mitochondria. Nucleic Acids Res 42, 499-508 (2014).
9. A. L. Borges et al., Widespread stop-codon recoding in bacteriophages may regulate translation of lytic genes. Nat Microbiol 7, 918-927 (2022).
10. M. A. Santos, A. C. Gomes, M. C. Santos, L. C. Carreto, G. R. Moura, The genetic code of the fungal CTG clade. C R Biol 334, 607-611 (2011).
11. D. J. Taylor, M. J. Ballinger, S. M. Bowman, J. A. Bruenn, Virus-host co-evolution under a modified nuclear genetic code. PeerJ 1, e50 (2013).
12. Y. Shulgina, S. R. Eddy, A computational screen for alternative genetic codes in over 250,000 genomes. Elife 10, (2021).
13. D. G. Gibson et al., Complete chemical synthesis, assembly, and cloning of a Mycoplasma genitalium genome. Science 319, 1215-1220 (2008).
14. D. G. Gibson et al., One-step assembly in yeast of 25 overlapping DNA fragments to form a complete synthetic Mycoplasma genitalium genome. Proc Natl Acad Sci USA 105, 20404-20409 (2008).
15. J. Fredens et al., Total synthesis of Escherichia coli with a recoded genome. Nature 569, 514-+(2019).
16. F. J. Isaacs et al., Precise manipulation of chromosomes in vivo enables genome-wide codon replacement. Science 333, 348-353 (2011).
17. M. J. Lajoie et al., Genomically recoded organisms expand biological functions. Science 342, 357-360 (2013).
18. W. E. Robertson et al., Sense codon reassignment enables viral resistance and encoded polymer synthesis. Science 372, 1057-1062 (2021).
19. G. Pines, J. D. Winkler, A. Pines, R. T. Gill, Refactoring the Genetic Code for Increased Evolvability. mBio 8, (2017).
20. J. Calles, I. Justice, D. Brinkley, A. Garcia, D. Endy, Fail-safe genetic codes designed to intrinsically contain engineered organisms. Nucleic Acids Res 47, 10439-10451 (2019).
21. M. Schmidt, V. Kubyshkin, How To Quantify a Genetic Firewall? A Polarity-Based Metric for Genetic Code Engineering. Chembiochem 22, 1268-1284 (2021).
22. D. S. Marks, S. W. Michnick, Democratizing the mapping of gene mutations to protein biophysics. Nature 604, 47-48 (2022).
23. S. Teng, A. K. Srivastava, C. E. Schwartz, E. Alexov, L. Wang, Structural assessment of the effects of amino acid substitutions on protein stability and protein protein interaction. Int J Comput Biol Drug Des 3, 334-349 (2010).
24. V. Parthiban, M. M. Gromiha, D. Schomburg, CUPSAT: prediction of protein stability upon point mutations. Nucleic Acids Res 34, W239-242 (2006).
25. P. C. Ng, S. Henikoff, Predicting the effects of amino acid substitutions on protein function. Annu Rev Genomics Hum Genet 7, 61-80 (2006).
26. B. A. Renda, M. J. Hammerling, J. E. Barrick, Engineering reduced evolutionary potential for synthetic biology. Mol Biosyst 10, 1668-1678 (2014).
27. G. Moratorio et al., Attenuation of RNA viruses by redirecting their evolution in sequence space. Nat Microbiol 2, 17088 (2017).
28. J. R. Coleman et al., Virus attenuation by genome-scale changes in codon pair bias. Science 320, 1784-1787 (2008).
29. P. W. Barone et al., Viral contamination in biologic manufacture and implications for emerging therapies. Nat Biotechnol 38, 563-572 (2020).
30. P. Alamos et al., Functionality of tRNAs encoded in a mobile genetic element from an acidophilic bacterium. RNA Biol 15, 518-527 (2018).
31. T. Tuller et al., Association between translation efficiency and horizontal gene transfer within microbial communities. Nucleic Acids Res 39, 4743-4755 (2011).
32. J. W. Lee, C. T. Y. Chan, S. Slomovic, J. J. Collins, Next-generation biocontainment systems for engineered organisms. Nat Chem Biol 14, 530-537 (2018).
33. W. E. Robertson et al., Creating custom synthetic genomes in Escherichia coli with REXER and GENESIS. Nat Protoc 16, 2345-2380 (2021).
34. K. C. Murphy, lambda Recombination and Recombineering. EcoSal Plus 7, (2016).
35. K. Wang et al., Defining synonymous codon compression schemes by genome recoding. Nature 539, 59-64 (2016).

1. A cell that:
comprises a genome wherein at least a first type of sense codon has been recoded such that a first endogenous tRNA is dispensable;
does not express the first endogenous tRNA;
expresses a first modified tRNA capable of decoding the first type of sense codon, wherein the first modified tRNA is charged with a first amino acid that is not a naturally cognate amino acid for the first type of sense codon; and
comprises a gene required for viability, wherein the gene comprises at least one occurrence of the first type of sense codon and the cell is viable when the first type of sense codon in said gene is decoded as the first amino acid.

2. The cell of claim 1, wherein the first modified tRNA is an anticodon-swapped tRNA canonically associated with the first amino acid.

3. The cell of claim 1, wherein the first modified tRNA is derived from a tRNA that is endogenous to the cell and is an isoacceptor for the first amino acid, or is derived from a tRNA found in a mobile genetic element and is an isoacceptor for the first amino acid.

4. The cell of claim 1, wherein the cell is not viable if the first type of sense codon in the gene required for viability is decoded according to the canonical genetic code, or wherein the first type of sense codon in the gene required for viability at least partially contributes to a loss of viability if decoded according to the canonical genetic code.

5. The cell of claim 1, wherein the gene required for viability is an essential gene or a positive selectable marker.

6. The cell of claim 1, wherein the first amino acid is a naturally occurring amino acid.

7. The cell of claim 1, wherein a second type of sense codon has been recoded within the genome.

8. The cell of claim 7, wherein a second endogenous tRNA is dispensable and the cell does not express the second endogenous tRNA.

9. The cell of claim 7, wherein the cell expresses a second modified tRNA, which is capable of decoding the second type of sense codon, wherein the second modified tRNA is charged with a second amino acid that is not a naturally cognate amino acid for the second type of sense codon.

10. The cell of claim 9, wherein a gene required for viability comprises at least one occurrence of the second type of sense codon and the cell is viable when the second type of sense codon in said gene is decoded as the second amino acid.

11. The cell of claim 10, wherein the cell is not viable if the second type of sense codon in the gene required for viability is decoded according to the canonical genetic code, or wherein the second type of sense codon at least partially contributes to a loss of viability if decoded according to the canonical genetic code.

12. The cell of claim 9, wherein the second modified tRNA:
(a) is an anticodon-swapped tRNA canonically associated with the second amino acid; or
(b) is derived from a tRNA that is endogenous to the cell and is an isoacceptor for the second amino acid, or is derived from a tRNA found in a mobile genetic element and is an isoacceptor for the second amino acid.

13. (canceled)

14. The cell of claim 9, wherein the second amino acid is a naturally occurring amino acid.

15. The cell of claim 9, wherein the first and the second amino acids are the same type of amino acid or are different types of amino acid.

16. The cell of claim 7, wherein:
(a) the first type of sense codon is TCA and the second type of sense codon is TCG; or
(b) the first type of sense codon is TCA or TCG.

17. (canceled)

18. The cell of claim 1, wherein the cell is viable when its genes are decoded by the modified tRNA(s) and is non-viable when its genes are decoded at least partially according to the canonical genetic code.

19. A cell that:
comprises a genome wherein a first type of sense codon and a second type of sense codon have been recoded such that a first endogenous tRNA and a second endogenous tRNA are dispensable;
does not express the first endogenous tRNA and the second endogenous tRNA;
expresses a first anticodon-swapped tRNA derived from a naturally occurring first parent tRNA, wherein the first anticodon-swapped tRNA is charged with a first amino acid and the first parent tRNA is an isoacceptor for the first amino acid, and wherein the first amino acid is not a naturally cognate amino acid for the first type of sense codon; and
expresses a second anticodon-swapped tRNA derived from a naturally occurring second parent tRNA, wherein the second anticodon-swapped tRNA is charged with a second amino acid and the second parent tRNA is an isoacceptor for the second amino acid, and wherein the second amino acid is not a naturally cognate amino acid for the second type of sense codon;
wherein the first and/or second modified tRNA cannot decode any type of codon apart from the first type of sense codon and/or the second type of sense codon.

20. The cell of claim 19, wherein:
(a) the first and the second type of sense codon canonically encode the same amino acid;
(b) the first and the second type of sense codon would canonically be decoded by the same tRNA or overlapping tRNAs due to wobble base pairing, wherein the first anticodon-swapped tRNA cannot decode any codon type apart from the first type of sense codon and/or the second anticodon-swapped tRNA cannot decode any codon type apart from the second type of sense codon;
(c) the first and the second type of sense codon are of the formula XXN, and wherein the first anticodon-swapped tRNA cannot decode the second type of sense codon, and the second anticodon-swapped tRNA cannot decode the first type of sense codon;
(d) the first and the second type of sense codon canonically encode serine, the first and the second type of sense codon canonically encode alanine, or the first and the second type of sense codon canonically encode leucine; or
(e) the first type of sense codon is TCA and/or the second type of sense codon is TCG.

21. (canceled)

22. (canceled)

23. The cell of claim 19, wherein;
(a) the first amino acid and the second amino acid are different types of amino acids;
(b) the first and/or second amino acid is a naturally occurring amino acid.

24. The cell of claim 19, wherein the first and second parent tRNAs are derived from the same cell type as the cell of claim 19.

25. The cell of claim 19, wherein the first and/or second anticodon-swapped tRNA;
(a) comprise identity elements that are recognised by an aminoacyl-tRNA synthetase endogenous to the cell; or
(b) does not decode TCC or TCT codons.

26-29. (canceled)

30. The cell of claim 19, wherein the first amino acid is any one of alanine, histidine, leucine, and proline; and/or the second amino acid is any one of alanine, histidine, leucine, and proline.

31. The cell of claim 19, wherein the first and/or second anticodon-swapped tRNA is derived from a parent tRNA encoded by ArgQ, ArgU, GltU, HisR, ProK, ProL, ProM, TrpT, ThrU, ThrT, TyrU, TyrV, AlaT, or LeuQ.

32. The cell of claim 31, wherein the first and/or second anticodon-swapped tRNA is derived from a parent tRNA encoded by HisR, ProM, AlaT, or LeuQ.

33. The cell of claim 19, wherein the parent tRNA is an E. coli tRNA.

34. A cell that:
comprises a genome wherein a first type of sense codon and a second type of sense codon have been recoded such that a first endogenous tRNA and a second endogenous tRNA are dispensable;
does not express the first endogenous tRNA and the second endogenous tRNA;
expresses a first modified tRNA capable of decoding the first type of sense codon, wherein the first modified tRNA is charged with a first amino acid that is not a naturally cognate amino acid for the first type of sense codon; and
expresses a second modified tRNA capable of decoding the second type of sense codon, wherein the second modified tRNA is charged with a second amino acid that is not a naturally cognate amino acid for the second type of sense codon;
wherein:
i) the first amino acid is alanine and the second amino acid is alanine;
ii) the first amino acid is alanine and the second amino acid is histidine;
iii) the first amino acid is alanine and the second amino acid is leucine;
iv) the first amino acid is alanine and the second amino acid is proline;
v) the first amino acid is histidine and the second amino acid is alanine;
vi) the first amino acid is histidine and the second amino acid is histidine;
vii) the first amino acid is histidine and the second amino acid is leucine;
viii) the first amino acid is histidine and the second amino acid is proline;
ix) the first amino acid is leucine and the second amino acid is alanine;
x) the first amino acid is leucine and the second amino acid is histidine;
xi) the first amino acid is leucine and the second amino acid is proline;
xii) the first amino acid is proline and the second amino acid is alanine;
xiii) the first amino acid is proline and the second amino acid is histidine;
xiv) the first amino acid is proline and the second amino acid is leucine; or
xv) the first amino acid is proline and the second amino acid is proline.

35. The cell of claim 34, wherein;
(a) the first modified tRNA cannot decode the second type of sense codon and/or the second modified tRNA cannot decode the first type of sense codon; or
(b) the first modified tRNA cannot decode any type of codon apart from the first type of sense codon and/or the second modified tRNA cannot decode any type of codon apart from the second type of sense codon.

36. (canceled)

37. The cell of claim 34, wherein the first modified tRNA is an anticodon-swapped tRNA canonically associated with the first amino acid, and/or the second modified tRNA is an anticodon-swapped tRNA canonically associated with the second amino acid.

38. The cell of claim 34, wherein the first modified tRNA is derived from a tRNA that is endogenous to the cell and is an isoacceptor for the first amino acid, or is derived from a tRNA found in a mobile genetic element and is an isoacceptor for the first amino acid; and/or the second modified tRNA is derived from a tRNA that is endogenous to the cell and is an isoacceptor for the second amino acid, or is derived from a tRNA found in a mobile genetic element and is an isoacceptor for the second amino acid.

39. The cell of claim 34, wherein the first and/or second modified tRNA comprise identity elements that are recognised by an aminoacyl-tRNA synthetase endogenous to the cell.

40. The cell of claim 34, wherein:
(a) the first and the second type of sense codon canonically encode serine; or
(b) the first type of sense codon is TCA and/or the second type of sense codon is TCG.

41. (canceled)

42. The cell of claim 7, wherein the essential genes of the genome do not contain naturally occurring instances of the second type of sense codon, and the second endogenous tRNA is a cognate tRNA for the second type of sense codon.

43. The cell of claim 7, wherein the genome comprises 10, 9, 8, 7, 6, 5, 4, 3, 2, 1, or no naturally occurring instances of the second type of sense codon, and the second endogenous tRNA is a cognate tRNA for the second type of sense codon.

44. The cell of claim 7, wherein the second type of sense codon is TCG and the second endogenous tRNA is tRNASerCGA.

45. The cell of claim 1, wherein the essential genes of the genome do not contain naturally occurring instances of the first type of sense codon, and the first endogenous tRNA is a cognate tRNA for the first type of sense codon.

46. The cell of claim 1, wherein the genome comprises 10, 9, 8, 7, 6, 5, 4, 3, 2, 1, or no naturally occurring instances of the first type of sense codon, and the first endogenous tRNA is a cognate tRNA for the first type of sense codon.

47. The cell of claim 1, wherein the first type of sense codon is TCA and the first endogenous tRNA is tRNASerUGA or the first type of sense codon is TCG and the first endogenous is tRNASerCGA.

48. The cell of claim 1, wherein a plurality of naturally occurring instances of the TCA codon have been replaced with AGT and/or a plurality of naturally occurring instances of the TCG codon have been replaced with AGC.

49. The cell of claim 1, wherein the cell has increased resistance to horizontal gene transfer or mobile genetic elements.

50. The cell of claim 1, wherein the cell's genome is at least 80%, 85%, 90%, 95%, 98%, 99%, 99.5%, 99.8%, 99.9%, or 100% identical to any one of SEQ ID NOs: 1 to 6.

51. The cell of claim 1, wherein the cell is prokaryotic cell, a bacterial cell, or an Escherichia coli cell.

52. A method of increasing the resistance of a cell to mobile genetic elements or horizontal gene transfer, wherein the cell has been modified to reassign at least one type of sense codon to an amino acid not associated with the sense codon in the canonical genetic code, said method comprising:
modifying a gene required for viability to include at least one occurrence of the reassigned sense codon, wherein
the cell is viable if the reassigned sense codon in said gene is decoded as the reassigned amino acid, and
the cell is not viable if the reassigned sense codon in said gene is decoded according to the canonical genetic code, or wherein the reassigned sense codon in said gene at least partially contributes to a loss of viability if decoded according to the canonical genetic code.

53-59. (canceled)

60. A method of altering susceptibility of a gene to mutations that alter the encoded amino acid sequence, the method comprising:
i) identifying a target gene; and
ii) incubating a cell comprising the target gene, wherein the cell comprises a tRNA capable of decoding at least one sense codon to a reassigned amino acid, wherein the cell is according to claim 1.

61-64. (canceled)

65. A method for making a polymer, the method comprising:
culturing a cell according to claim 1,
providing the cell with a nucleic acid sequence encoding the polymer, and
obtaining the polymer.
